SEQUENCE LISTING (106 sequences)

SEQ ID NO: 1 - protein, 9 aa, Homo sapiens
RMLLSHTGK

SEQ ID NO: 2 - protein, 9 aa, Homo sapiens
LPHRALSGI

SEQ ID NO: 3 - protein, 9 aa, Homo sapiens
GTNPTAAVK

SEQ ID NO: 4 - protein, 9 aa, Homo sapiens
LRHKLVLNR

SEQ ID NO: 5 - protein, 9 aa, Homo sapiens
RIGGVGVEK

SEQ ID NO: 6 - protein, 9 aa, Homo sapiens
SIIETVNSL

SEQ ID NO: 7 - protein, 10 aa, Homo sapiens
TVNTQQYNTK

SEQ ID NO: 8 - protein, 9 aa, Homo sapiens
SVSHLHIFF

SEQ ID NO: 9 - protein, 9 aa, Homo sapiens
TTLENLPQK

SEQ ID NO: 10 - protein, 9 aa, Homo sapiens
GQIELSIYR

SEQ ID NO: 11 - protein, 9 aa, Homo sapiens
HGALSIRSI

SEQ ID NO: 12 - protein, 8 aa, Homo sapiens
RLMKFLPV

SEQ ID NO: 13 - protein, 9 aa, Homo sapiens
SLYISEERK

SEQ ID NO: 14 - protein, 8 aa, Homo sapiens
AQKLQVRI

SEQ ID NO: 15 - protein, 8 aa, Homo sapiens
VQTAVLNV

SEQ ID NO: 16 - protein, 9 aa, Homo sapiens
KIGEVIVTK

SEQ ID NO: 17 - protein, 9 aa, Homo sapiens
TRSTIILHL

SEQ ID NO: 18 - protein, 9 aa, Homo sapiens
SILRGSLGK

SEQ ID NO: 19 - protein, 9 aa, Homo sapiens
RLVSNRVVR

SEQ ID NO: 20 - protein, 9 aa, Homo sapiens
SLKNLEPIK

SEQ ID NO: 21 - protein, 9 aa, Homo sapiens
RSAGPRAPL

SEQ ID NO: 22 - protein, 10 aa, Homo sapiens
VLYRSVLLLK

SEQ ID NO: 23 - protein, 9 aa, Homo sapiens
KAAGAGAAK

SEQ ID NO: 24 - protein, 9 aa, Homo sapiens
RNRQVATAL

SEQ ID NO: 25 - protein, 9 aa, Homo sapiens
VEAPHLPSF

SEQ ID NO: 26 - protein, 9 aa, Homo sapiens
EYNSDLHQF

SEQ ID NO: 27 - protein, 10 aa, Homo sapiens
TYKYVDINTF

SEQ ID NO: 28 - protein, 8 aa, Homo sapiens
VIDEPVRL

SEQ ID NO: 29 - protein, 9 aa, Homo sapiens
RYLEKFYGL

SEQ ID NO: 30 - protein, 9 aa, Homo sapiens
KRLPSPSLF

SEQ ID NO: 31 - protein, 9 aa, Homo sapiens
KSINEFWNK

SEQ ID NO: 32 - protein, 10 aa, Homo sapiens
RVFHGNETTK

SEQ ID NO: 33 - protein, 9 aa, Homo sapiens
RIQLPVVSK

SEQ ID NO: 34 - protein, 9 aa, Homo sapiens
SIFRTPISK

SEQ ID NO: 35 - protein, 9 aa, Homo sapiens
AEYHSQSRA

SEQ ID NO: 36 - protein, 9 aa, Homo sapiens
IHNSDQILL

SEQ ID NO: 37 - protein, 9 aa, Homo sapiens
KMHQSRISY

SEQ ID NO: 38 - protein, 9 aa, Homo sapiens
QMAGLRDTY

SEQ ID NO: 39 - protein, 11 aa, Homo sapiens
RIKNWGSGMTF

SEQ ID NO: 40 - protein, 10 aa, Homo sapiens
AQYDQASTKY

SEQ ID NO: 41 - protein, 9 aa, Homo sapiens
FVDNQYWRY

SEQ ID NO: 42 - protein, 10 aa, Homo sapiens
QLDSAVKNLY

SEQ ID NO: 43 - protein, 8 aa, Homo sapiens
EQLKVTTI

SEQ ID NO: 44 - protein, 9 aa, Homo sapiens
FRSWAVQTF

SEQ ID NO: 45 - protein, 9 aa, Homo sapiens
KLYQSIIGY

SEQ ID NO: 46 - protein, 9 aa, Homo sapiens
RVAKTNSLR

SEQ ID NO: 47 - protein, 9 aa, Homo sapiens
SANVSKVSF

SEQ ID NO: 48 - protein, 9 aa, Homo sapiens
SQHDILQMI

SEQ ID NO: 49 - protein, 9 aa, Homo sapiens
VAKTPRNTY

SEQ ID NO: 50 - protein, 9 aa, Homo sapiens
RPRLSAILF

SEQ ID NO: 51 - DNA, 27 bp, Homo sapiens
  source 1..27: mol_type = other DNA
aggatgttactgagccacacaggaaaa

SEQ ID NO: 52 - DNA, 27 bp, Homo sapiens
  source 1..27: mol_type = other DNA
cttcctcacagggctttgtcaggcatt

SEQ ID NO: 53 - DNA, 27 bp, Homo sapiens
  source 1..27: mol_type = other DNA
ggcaccaaccccacggctgcagtgaag

SEQ ID NO: 54 - DNA, 27 bp, Homo sapiens
  source 1..27: mol_type = other DNA
ttaaggcataagttagttctgaacaga

SEQ ID NO: 55 - DNA, 27 bp, Homo sapiens
  source 1..27: mol_type = other DNA
cggatagggggagtaggagtagagaag

SEQ ID NO: 56 - DNA, 27 bp, Homo sapiens
  source 1..27: mol_type = other DNA
tctataatagaaactgtaaattctctt

SEQ ID NO: 57 - DNA, 30 bp, Homo sapiens
  source 1..30: mol_type = other DNA
actgtcaacactcaacaatataatacaaaa

SEQ ID NO: 58 - DNA, 27 bp, Homo sapiens
  source 1..27: mol_type = other DNA
tctgttagccatttgcatatcttcttt

SEQ ID NO: 59 - DNA, 27 bp, Homo sapiens
  source 1..27: mol_type = other DNA
accacactggaaaacctgccccagaag

SEQ ID NO: 60 - DNA, 24 bp, Homo sapiens
  source 1..24: mol_type = other DNA
gcacaaaaattgcaagtgaggatt

SEQ ID NO: 61 - DNA, 27 bp, Homo sapiens
  source 1..27: mol_type = other DNA
ggtcaaatagagttatcaatatatagg

SEQ ID NO: 62 - DNA, 27 bp, Homo sapiens
  source 1..27: mol_type = other DNA
catggtgccctgtccataagaagtatt

SEQ ID NO: 63 - DNA, 24 bp, Homo sapiens
  source 1..24: mol_type = other DNA
agactgatgaaattccttccagtt

SEQ ID NO: 64 - DNA, 27 bp, Homo sapiens
  source 1..27: mol_type = other DNA
tctctgtatatctcagaagaaaggaaa

SEQ ID NO: 65 - DNA, 24 bp, Homo sapiens
  source 1..24: mol_type = other DNA
gttcaaactgctgttttaaatgtg

SEQ ID NO: 66 - DNA, 27 bp, Homo sapiens
  source 1..27: mol_type = other DNA
aaaattggagaggtcattgtgaccaaa

SEQ ID NO: 67 - DNA, 27 bp, Homo sapiens
  source 1..27: mol_type = other DNA
acccgcagcaccatcatcctgcacctc

SEQ ID NO: 68 - DNA, 27 bp, Homo sapiens
  source 1..27: mol_type = other DNA
tcgattttacgtggatctctagggaaa

SEQ ID NO: 69 - DNA, 27 bp, Homo sapiens
  source 1..27: mol_type = other DNA
agattggtttcaaatagagtagtcagg

SEQ ID NO: 70 - DNA, 27 bp, Homo sapiens
  source 1..27: mol_type = other DNA
agcctgaaaaaccttgagccaattaaa

SEQ ID NO: 71 - DNA, 27 bp, Homo sapiens
  source 1..27: mol_type = other DNA
cgctctgcgggtccgcgggcccctctc

SEQ ID NO: 72 - DNA, 30 bp, Homo sapiens
  source 1..30: mol_type = other DNA
gtgttgtaccgttcggtacttttactgaaa

SEQ ID NO: 73 - DNA, 27 bp, Homo sapiens
  source 1..27: mol_type = other DNA
aaggctgccggcgccggcgctgctaag

SEQ ID NO: 74 - DNA, 27 bp, Homo sapiens
  source 1..27: mol_type = other DNA
aggaataggcaggttgctactgctctg

SEQ ID NO: 75 - DNA, 27 bp, Homo sapiens
  source 1..27: mol_type = other DNA
gtggaagcccctcatctgccttcattc

SEQ ID NO: 76 - DNA, 27 bp, Homo sapiens
  source 1..27: mol_type = other DNA
gagtacaacagtgacctccatcaattt

SEQ ID NO: 77 - DNA, 30 bp, Homo sapiens
  source 1..30: mol_type = other DNA
acctacaaatatgttgacatcaacacattt

SEQ ID NO: 78 - DNA, 24 bp, Homo sapiens
  source 1..24: mol_type = other DNA
gttattgatgagcctgtacggcta

SEQ ID NO: 79 - DNA, 27 bp, Homo sapiens
  source 1..27: mol_type = other DNA
agatacttagaaaaattttatggcctt

SEQ ID NO: 80 - DNA, 27 bp, Homo sapiens
  source 1..27: mol_type = other DNA
aaaaggttgccatcaccatctctgttt

SEQ ID NO: 81 - DNA, 27 bp, Homo sapiens
  source 1..27: mol_type = other DNA
aaaagcataaatgaattttggaataaa

SEQ ID NO: 82 - DNA, 30 bp, Homo sapiens
  source 1..30: mol_type = other DNA
cgtgtctttcatggtaatgaaactaccaaa

SEQ ID NO: 83 - DNA, 27 bp, Homo sapiens
  source 1..27: mol_type = other DNA
cgtatccagcttcctgttgtgtcaaaa

SEQ ID NO: 84 - DNA, 27 bp, Homo sapiens
  source 1..27: mol_type = other DNA
tcaatattcagaactccaatttctaaa

SEQ ID NO: 85 - DNA, 27 bp, Homo sapiens
  source 1..27: mol_type = other DNA
gcagagtatcattctcaaagtagggct

SEQ ID NO: 86 - DNA, 27 bp, Homo sapiens
  source 1..27: mol_type = other DNA
atacataactctgaccagatcctacta

SEQ ID NO: 87 - DNA, 27 bp, Homo sapiens
  source 1..27: mol_type = other DNA
aaaatgcatcagtcaagaataagttac

SEQ ID NO: 88 - DNA, 27 bp, Homo sapiens
  source 1..27: mol_type = other DNA
cagatggcgggactaagagatacctac

SEQ ID NO: 89 - DNA, 33 bp, Homo sapiens
  source 1..33: mol_type = other DNA
aggatcaaaaactggggcagcgggatgactttc

SEQ ID NO: 90 - DNA, 29 bp, Homo sapiens
  source 1..29: mol_type = other DNA
gcacaatacgaccaggcgtcaaccaagta

SEQ ID NO: 91 - DNA, 27 bp, Homo sapiens
  source 1..27: mol_type = other DNA
tttgtagataaccagtattggaggtat

SEQ ID NO: 92 - DNA, 30 bp, Homo sapiens
  source 1..30: mol_type = other DNA
caactggactctgctgtaaagaacctgtac

SEQ ID NO: 93 - DNA, 24 bp, Homo sapiens
  source 1..24: mol_type = other DNA
gagcagctaaaagtaacaacaatt

SEQ ID NO: 94 - DNA, 27 bp, Homo sapiens
  source 1..27: mol_type = other DNA
ttccgcagctgggccgtgcaaaccttc

SEQ ID NO: 95 - DNA, 27 bp, Homo sapiens
  source 1..27: mol_type = other DNA
aagttataccagagtataataggatac

SEQ ID NO: 96 - DNA, 27 bp, Homo sapiens
  source 1..27: mol_type = other DNA
cgggtggccaagaccaactccttacgg

SEQ ID NO: 97 - DNA, 27 bp, Homo sapiens
  source 1..27: mol_type = other DNA
agtgccaacgtttcaaaagtttctttt

SEQ ID NO: 98 - DNA, 27 bp, Homo sapiens
  source 1..27: mol_type = other DNA
agtcaacatgatattttacagatgata

SEQ ID NO: 99 - DNA, 27 bp, Homo sapiens
  source 1..27: mol_type = other DNA
gtagcaaaaacgcccaggaacacctac

SEQ ID NO: 100 - DNA, 27 bp, Homo sapiens
  source 1..27: mol_type = other DNA
cggccacggctcagtgctattctcttt

SEQ ID NO: 101 - protein, 9 aa, Homo sapiens
RLASRPLLL

SEQ ID NO: 102 - protein, 9 aa, Homo sapiens
YRNSYEIEY

SEQ ID NO: 103 - protein, 9 aa, Homo sapiens
APTPARPVL

SEQ ID NO: 104 - protein, 10 aa, Homo sapiens
RLAEPSQMLK

SEQ ID NO: 105 - protein, 11 aa, Homo sapiens
SPKATGVFTTL

SEQ ID NO: 106 - protein, 9 aa, Homo sapiens
SVLTQPPSV

(19) United States
(12) Patent Application Publication
(10) Pub. No.: US 2025/0136937 A1
(43) Pub. Date: May 1, 2025

(54) NOVEL TUMOR-SPECIFIC ANTIGENS FOR COLORECTAL CANCER AND USES THEREOF

(71) Applicant: UNIVERSITÉ DE MONTRÉAL, Montréal (CA)

(72) Inventors: Claude PERREAULT, Montréal (CA); Pierre THIBAULT, Montreal (CA); Jenna CLEYLE, Fort McMurray (CA); Marie-pierre HARDY, Terrebonne (CA)

(21) Appl. No.: 18/692,730

(22) PCT Filed: Sep. 16, 2022
(86) PCT No.: PCT/CA2022/051377

§ 371 Date: Mar. 15, 2024

Related U.S. Application Data
(60) Provisional application No. 63/261,315, filed on Sep. 17, 2021.

(51) Int. Cl. C12N 5/0783; A61K 39/00; A61K 45/06; A61P 35/00; C07K 7/06; C07K 14/725; C07K 16/28; C12N 15/11

(52) U.S. Cl. CPC C12N 5/0638; A61K 39/0011; A61K 45/06; A61P 35/00; C07K 7/06; C07K 14/7051; C07K 16/2809; C12N 15/11

(57) ABSTRACT

Colorectal cancer (CRC) has not benefited from innovative immunotherapies, mainly because of the lack of actionable immune targets. Novel tumor-specific antigens (TSAs) and tumor-associated antigens (TAAs) expressed by CRC cells are described herein. Most of the TSAs described herein derives from aberrantly expressed unmutated genomic sequences, such as intronic and intergenic sequences, which are not expressed in normal tissues. Nucleic acids, compositions, cells, antibodies and vaccines derived from these TSAs are described. The use of the TSAs, nucleic acids, compositions, antibodies, cells and vaccines for the treatment of CRC is also described.

### CROSS REFERENCE TO RELATED APPLICATIONS

The present application claims the benefits of U.S. provisional patent application No. 63/261,315 filed on Sep. 17, 2021, which is incorporated herein by reference.

### TECHNICAL FIELD

The present disclosure generally relates to the field of oncology, and more particularly to the treatment of cancer such as colorectal cancer.

### BACKGROUND ART

Colorectal cancer (CRC) is the third most commonly diagnosed cancer and the second leading cause of cancer death worldwide, with over 1.8 million cases and 881,000 deaths estimated in 2018 alone (1). The incidence of CRC is expected to increase as global socioeconomic changes occur, with a predicted 2.2 million cases and 1.1 million deaths occurring annually by 2030 (1,2). This significant disease burden highlights the necessity of developing new and effective treatments against this disease.

The positive correlation between the abundance of tumor-infiltrating lymphocytes (TILs) and increased overall survival in both colon and rectal cancer suggests that T cells can recognize biologically relevant tumor antigens in these tumors (3,4). The potential immunogenicity of these antigens made immune checkpoint inhibition (ICI) a promising treatment for cancer patients; however, early clinical trials evaluating their efficacy in CRC have yielded mixed results. Colorectal tumors characterized by deficiencies in mismatch repair proteins resulting in the accumulation of repetitive DNA sequences (microsatellites), known as microsatellite instability (MSI), have shown relative success in phase II clinical trials with anti-PD1 treatment (5). In contrast, such treatments have had very little efficacy in clinical trials against microsatellite stable (MSS) tumors that do not possess a high mutational burden, which make up approximately 80% of CRC cases (5,6).

Given the significance of the immune response in CRC and the limited success of ICI alone, a promising avenue for research in recent years has been a neoantigen-based vaccine or T cell receptor-based therapy which could be used with or without ICI and would ideally bridge the gap in treatment efficacy across MSI and MSS tumors. For example, tumor-associated antigens (TAAs), antigens overexpressed in cancer cells compared to normal cells, have been previously identified in CRC (7,8). While several TAAs have been tested in vaccine and phase I trials against CRC, most were met with ‘limited success’, likely due to the negative selection of such antigens by the thymus (9). In one study, treatment of metastatic CRC with genetically engineered anti-CEA T cells resulted in tumor regression in one patient but ‘serious inflammatory colitis’ in all patients, demonstrating that an adverse autoimmune response is another possible negative consequence of using TAAs (10).

Due to the mixed responses to TAAs, an effective neoantigen-based therapy would more likely utilize tumor-specific antigens (TSAs), which may be generated through genetic, epigenetic, and post-translational variations, including but not limited to single-nucleotide variants, aberrantly expressed transcripts, or novel splicing events, and are expressed exclusively by the tumor (11). The high prevalence of single nucleotide variants, splice variants, and INDEL mutations in CRC suggests that there is a high probability of unique antigens being presented by the major histocompatibility complex (MHC) of tumors, such that it would be possible to invoke a tumor-specific immune response (12). TSAs have recently been identified in CRC and have demonstrated some success in phase I and II vaccine trials. A 2015 vaccine trial using frameshift antigens originating from MSI-high tumors demonstrated significant and specific immune responses among all patients (13). However, as this study used antigens derived from microsatellite instability frameshifts, these findings are not applicable to the majority of CRC patients. Other studies identifying TSAs in CRC to date have focused exclusively on mutant TSAs (mTSAs) derived from coding regions of the genome (13,14). An investigation of MSS CRC organoids revealed that only 0.5% of non-silent mutations were identified as mTSAs; this was a significantly lower proportion than what was predicted by HLA-binding prediction software (15). In addition, previous studies did not employ mass spectrometry (MS) techniques to quantify the expression of those TSAs on tumor cells, which is information that could influence the therapeutic potential of targeting a given TSA (13,14).

In view of this, there is a pressing need to identify the tumor-specific antigens that can elicit therapeutic immune responses again CRC. Such antigens could be used as vaccines (± immune checkpoint inhibitors) or as targets for T-cell receptor-based approaches (cell therapy, bispecific biologics).

The present description refers to a number of documents, the content of which is herein incorporated by reference in their entirety.

### SUMMARY

In various aspects and embodiments, the present disclosure provides the following items 1 to 62:
1. A tumor antigen peptide (TAP) comprising or consisting of one of the following amino acid sequences:

| Sequence | SEQ ID NO: |
|---|---|
| SIIETVNSL | 6 |
| RMLLSHTGK | 1 |
| LPHRALSGI | 2 |
| GTNPTAAVK | 3 |
| LRHKLVLNR | 4 |
| RIGGVGVEK | 5 |
| TVNTQQYNTK | 7 |
| SVSHLHIFF | 8 |
| TTLENLPQK | 9 |
| AQKLQVRI | 10 |
| GQIELSIYR | 11 |
| HGALSIRSI | 12 |
| RLMKFLPV | 13 |
| SLYISEERK | 14 |
| VQTAVLNV | 15 |
| KIGEVIVTK | 16 |
| TRSTIILHL | 17 |
| SILRGSLGK | 18 |
| RLVSNRVVR | 19 |
| SLKNLEPIK | 20 |
| RSAGPRAPL | 21 |
| VLYRSVLLLK | 22 |
| KAAGAGAAK | 23 |

or a nucleic acid encoding said TAP.
2. The TAP or nucleic acid of item 1, wherein the TAP comprises one of the sequences defined in SEQ ID NO: 6, 1-5 and 6-17.
3. The TAP or nucleic acid of item 1 or 2, which binds to an HLA-A*02:01 molecule and comprises the sequence of SEQ ID NO: 6.
4. The TAP or nucleic acid of item 1 or 2, which binds to an HLA-A*03:01 molecule and comprises the sequence of SEQ ID NO:1, 11, or 14.
5. The TAP or nucleic acid of item 1 or 2, which binds to an HLA-A*03:02 molecule and comprises the sequence of SEQ ID NOs: 3, 5, 7, 16 or 23.
6. The TAP or nucleic acid of item 1 or 2, which binds to an HLA-A*11:01 molecule and comprises the sequence of SEQ ID NO:9 or 18.
7. The TAP or nucleic acid of item 1 or 2, which binds to an HLA-A*30:01 molecule and comprises the sequence of SEQ ID NO: 19, 20 or 23.
8. The TAP or nucleic acid of item 1 or 2, which binds to an HLA-A*32:01 molecule and comprises the sequence of SEQ ID NO:8.
9. The TAP or nucleic acid of item 1 or 2, which binds to an HLA-B*07:02 molecule and comprises the sequence of SEQ ID NO: 2 or 21.
10. The TAP or nucleic acid of item 1 or 2, which binds to an HLA-B*13:02 molecule and comprises the sequence of SEQ ID NO: 13.
11. The TAP or nucleic acid of item 1 or 2, which binds to an HLA-B*27:05 molecule and comprises the sequence of SEQ ID NO: 4.
12. The TAP or nucleic acid of item 1 or 2, which binds to an HLA-B*52:01 molecule and comprises the sequence of SEQ ID NO: 10, 12, or 15.
13. The TAP or nucleic acid of item 1 or 2, which binds to an HLA-C*06:02 molecule and comprises the sequence of SEQ ID NO: 17.
14. The TAP or nucleic acid of any one of items 1-13, wherein the TAP is encoded by a sequence located a non-protein coding region of the genome.
15. The TAP or nucleic acid of item 14, wherein said non-protein coding region of the genome is an untranslated transcribed region (UTR).
16. The TAP or nucleic acid of item 14, wherein said non-protein coding region of the genome is an intron.
17. The TAP or nucleic acid of item 14, wherein said non-protein coding region of the genome is an intergenic region.
18. The TAP or nucleic acid of item 14, wherein said non-protein coding region of the genome is a long non-coding RNAs.
19. The nucleic acid of any one of items 1 to 18, wherein the nucleic acid is an mRNA.
20. The nucleic acid of any one of items 1 to 18, wherein the nucleic acid is a DNA.
21. The nucleic acid of any one of items 1 to 20, wherein the nucleic acid is a component of a viral vector.
22. A combination comprising at least two of the TAPs or nucleic acids defined in any one of items 1-21.
23. A synthetic long peptide (SLP) comprising at least one of the amino acid sequences defined in item 1.
24. A vesicle or particle comprising the TAP, nucleic acid, combination or SLP of any one of items 1 to 23.
25. The vesicle or particle of item 24, wherein the vesicle is a lipid nanoparticle (LNP).
26. The vesicle or particle of item 24 or 25, which comprises a cationic lipid.
27. A composition comprising the TAP, nucleic acid, combination or SLP of any one of items 1 to 23, or the vesicle or particle of any one of items 24-26, and a pharmaceutically acceptable carrier.
28. A vaccine comprising the TAP, nucleic acid, combination or SLP of any one of items 1 to 23, the vesicle or particle of any one of items 24-26, or the composition of item 27, and an adjuvant.
29. An isolated major histocompatibility complex (MHC) class I molecule comprising the TAP of any one of items 1-18 in its peptide binding groove.
30. The isolated MHC class I molecule of item 29, which is in the form of a multimer.
31. The isolated MHC class I molecule of item 30, wherein said multimer is a tetramer.
32. An isolated cell comprising (i) the TAP of any one of items 1-18, (ii) the combination of item 19; (iii) the SLP of item 23; or (iv) a vector comprising a nucleotide sequence encoding the TAP of any one of items 1-18, the combination of item 19 or the SLP of item 23.
33. An isolated cell expressing at its surface major histocompatibility complex (MHC) class I molecules comprising the TAP of any one of items 1-18 or the combination of item 19 in their peptide binding groove.
34. The cell of item 33, which is an antigen-presenting cell (APC).
35. The cell of item 34, wherein said APC is a dendritic cell.
36. A T-cell receptor (TCR) that specifically recognizes the isolated MHC class I molecule of any one of items 29-31 and/or MHC class I molecules expressed at the surface of the cell of any one of items 32-35.
37. An antibody or an antigen-binding fragment thereof that specifically binds to the isolated MHC class I molecule of any one of items 29-31 and/or MHC class I molecules expressed at the surface of the cell of any one of items 33-35.
38. The antibody or antigen-binding fragment thereof according to item 37, which is a bispecific antibody or antigen-binding fragment thereof.
39. The antibody or antigen-binding fragment thereof according to item 38, wherein the bispecific antibody or antigen-binding fragment thereof is a single-chain diabody (scDb).
40. The antibody or antigen-binding fragment thereof according to item 38 or 39, wherein the bispecific antibody or antigen-binding fragment thereof also specifically binds to a T cell signaling molecule.
41. The antibody or antigen-binding fragment thereof according to item 40, wherein the T cell signaling molecule is a CD3 chain.
42. An isolated cell expressing at its cell surface the TCR of item 36.
43. The isolated cell of item 42, which is a CD8⁺ T lymphocyte.
44. A cell population comprising at least 0.5% of the isolated cell as defined in item 42 or 43.
45. A method of treating cancer, such as colorectal cancer, in a subject comprising administering to the subject an effective amount of:
   (a) a TAP comprising or consisting of one of the amino acid sequences defined in SEQ ID NOs: 1-23 and 25-50, or a synthetic long peptide (SLP) comprising at least one of the sequences set forth in SEQ ID NOs: 1-23 and 25-50;
   (b) at least one nucleic acid encoding the TAP, combination thereof or SLP defined in (a);
   (c) a vesicle or particle comprising the TAP, combination thereof or SLP defined in (a) or the at least one nucleic acid defined in (b);
   (d) a composition comprising the TAP, combination thereof or SLP defined in (a), the at least one nucleic acid defined in (b), or the vesicle or particle defined in (c), and a pharmaceutically acceptable carrier;
   (e) a vaccine comprising the TAP, combination thereof or SLP defined in (a), the at least one nucleic acid defined in (b), the vesicle or particle defined in (c), or the composition defined in (d), and an adjuvant;
   (f) a cell expressing at its surface major histocompatibility complex (MHC) class I molecules comprising the TAP or combination thereof defined in (a) in their peptide binding groove;
   (g) a cell expressing at its cell surface a T-cell receptor (TCR) that specifically recognizes MHC class I molecules expressed at the surface of the cell defined in (f); or
   (h) a soluble TCR, an antibody or an antigen-binding fragment thereof that specifically binds to the MHC class I molecules expressed at the surface of the cell defined in (f).
46. The method of item 45, wherein the TAP or nucleic acid is as defined in any one of items 1 to 21, the combination is as defined in item 22, the SLP is as defined in item 23; the vesicle is as defined in any one of items 24-26, the composition is as defined in item 27, the vaccine is as defined in item 28, the cell is as defined in any one of items 32-35, 42 and 43, the cell population is as defined in item 44, and/or the antibody or antigen-binding fragment is as defined in any one of items 37-41.
47. The method of item 45 or 46, wherein the CRC is colon cancer.
48. The method of item 45 or 46, wherein the CRC is rectal cancer.
49. The method of any one of items 45-48, further comprising administering at least one additional antitumor agent or therapy to the subject.
50. The method of item 49, wherein said at least one additional antitumor agent or therapy is a chemotherapeutic agent, immunotherapy, an immune checkpoint inhibitor, radiotherapy or surgery.
51. Use of:
   (a) a TAP comprising or consisting of one of the amino acid sequences defined in SEQ ID NOs: 1-23 and 25-50, or a synthetic long peptide (SLP) comprising at least one of the sequences set forth in SEQ ID NOs: 1-23 and 25-50;
   (b) at least one nucleic acid encoding the TAP, combination thereof or SLP defined in (a);
   (c) a vesicle or particle comprising the TAP, combination thereof or SLP defined in (a) or the at least one nucleic acid defined in (b);
   (d) a composition comprising the TAP, combination thereof or SLP defined in (a), the at least one nucleic acid defined in (b), or the vesicle or particle defined in (c), and a pharmaceutically acceptable carrier;
   (e) a vaccine comprising the TAP, combination thereof or SLP defined in (a), the at least one nucleic acid defined in (b), the vesicle or particle defined in (c), or the composition defined in (d), and an adjuvant;
   (f) a cell expressing at its surface major histocompatibility complex (MHC) class I molecules comprising the TAP or combination thereof defined in (a) in their peptide binding
   (g) a cell expressing at its cell surface a T-cell receptor (TCR) that specifically recognizes MHC class I molecules expressed at the surface of the cell defined in (f); or
   (h) a soluble TCR, an antibody or an antigen-binding fragment thereof that specifically binds to the MHC class I molecules expressed at the surface of the cell defined in (f);
for treating cancer, such as colorectal cancer, in a subject, or for the manufacture of a medicament for treating cancer, such as colorectal cancer, in a subject.
52. The use of item 55, wherein the TAP or nucleic acid is as defined in any one of items 1 to 21, the combination is as defined in item 22, the SLP is as defined in item 23; the vesicle is as defined in any one of items 24-26, the composition is as defined in item 27, the vaccine is as defined in item 28, the cell is as defined in any one of items 32-35, 42 and 43, the cell population is as defined in item 44, and/or the antibody or antigen-binding fragment is as defined in any one of items 37-41.
53. The use of item 51 or 52, wherein the CRC is colon cancer.
54. The use of item 51 or 52, wherein the CRC is rectal cancer.
55. The use of any one of items 51-54, further comprising the use at least one additional antitumor agent or therapy to the subject.
56. The use of item 55, wherein said at least one additional antitumor agent or therapy is a chemotherapeutic agent, immunotherapy, an immune checkpoint inhibitor, radiotherapy or surgery.
57. An agent for use in treating cancer, such as colorectal cancer, in a subject, wherein the agent is:
   (a) a TAP comprising or consisting of one of the amino acid sequences defined in SEQ ID NOs: 1-23 and 25-50, or a synthetic long peptide (SLP) comprising at least one of the sequences set forth in SEQ ID NOs: 1-23 and 25-50;
   (b) at least one nucleic acid encoding the TAP, combination thereof or SLP defined in (a);
   (c) a vesicle or particle comprising the TAP, combination thereof or SLP defined in (a) or the at least one nucleic acid defined in (b);
   (d) a composition comprising the TAP, combination thereof or SLP defined in (a), the at least one nucleic acid defined in (b), or the vesicle or particle defined in (c), and a pharmaceutically acceptable carrier;
   (e) a vaccine comprising the TAP, combination thereof or SLP defined in (a), the at least one nucleic acid defined in (b), the vesicle or particle defined in (c), or the composition defined in (d), and an adjuvant;
   (f) a cell expressing at its surface major histocompatibility complex (MHC) class I molecules comprising the TAP or combination thereof defined in (a) in their peptide binding
   (g) a cell expressing at its cell surface a T-cell receptor (TCR) that specifically recognizes MHC class I molecules expressed at the surface of the cell defined in (f); or
   (h) a soluble TCR, an antibody or an antigen-binding fragment thereof that specifically binds to the MHC class I molecules expressed at the surface of the cell defined in (f).
58. The agent for use according to item 61, wherein the TAP or nucleic acid is as defined in any one of items 1 to 21, the combination is as defined in item 22, the SLP is as defined in item 23; the vesicle is as defined in any one of items 24-26, the composition is as defined in item 27, the vaccine is as defined in item 28, the cell is as defined in any one of items 32-35, 42 and 43, the cell population is as defined in item 44, and/or the antibody or antigen-binding fragment is as defined in any one of items 37-41.
59. The agent for use according to item 57 or 58, wherein the CRC is colon cancer.
60. The agent for use according to item 57 or 58, wherein the CRC is rectal cancer.
61. The agent for use according to any one of items 57-60, further comprising the use at least one additional antitumor agent or therapy to the subject.
62. The agent for use according to item 61, wherein said at least one additional antitumor agent or therapy is a chemotherapeutic agent, immunotherapy, an immune checkpoint inhibitor, radiotherapy or surgery.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 depicts the proteogenomic workflow for the discovery of tumor-specific antigens (TSAs) in both colorectal cancer (CRC)-derived cell lines and primary tumor samples. Samples generated from CRC- and normal intestine-derived cell lines, and matching primary tumor/normal adjacent tissue (NAT) biopsies obtained from six individuals were all processed for both RNA sequencing and major histocompatibility complex class I (MHC-I) immunoprecipitation (IP). RNA sequencing data were used for both the transcriptomic characterization of the samples and the generation of customized global cancer proteome databases. For each sample, the MHC-I associated peptides (MAPs) isolated via IP were identified via LC-MS/MS using the respective database. After validating both the identification and the tumor specificity of the TSA candidates, their therapeutic potentials were evaluated though the prediction of both their immunogenicity and inter-tumoral distribution. Figure created with BioRender.com.

FIGS. 2A-E show the transcriptomic profile of primary tumor/normal adjacent tissue CRC biopsies. FIG. 2A: Principal component analysis (PCA) of the top 500 varying genes of each tumor/NAT sample following paired-end RNA seq and gene readcount normalization with DESeq2. MSI tissues (as determined by MSISensor) are encircled. FIG. 2B: GO term analysis of genes up/downregulated in CRC tissues compared to their adjacent NAT. Genes submitted to GO term analysis were those with |log 2FC|>1 and that were found to be differentially regulated in all samples, using TPM normalized values. FIG. 2C: Bar graph showing the mean ESTIMATE immune score of MSS NAT, MSI NAT, MSS CRC, and MSI CRC, with standard deviation shown. FIG. 2D: Stacked bar graph showing the mean proportion of the transcriptome attributable to 5 distinct transcript biotypes in NAT vs. CRC samples, with the differences in the proportion of non-coding transcripts being statistically significant between NAT and CRC (p=0.0156). FIG. 2E: Scatterplots displaying the expression of non-coding RNA transcripts (left), SNV counts (middle) and INDEL counts (right) of MSS and MSI CRC tissues determined by SNPEff genomic annotation, with mean and standard error bars.

FIGS. 3A-E depict the results of the immunopeptidomic analysis of CRC-derived cell lines and tissues. FIG. 3A: Top panel: Stacked bar chart displaying the number of unique peptides identified in CRC cell lines, and a horizontal line indicating the average number of MAPs per cell line. Bottom panel: Scatterplot indicating the correlation between the number of unique MAPs identified in each cell line and the presentation of MHC I at the cell surface (Pearson's r=0.96). FIG. 3B: Stacked bar chart displaying the number of unique peptides identified in primary tissue samples, and a horizontal line indicating the average number of MAPs per tissue sample. ‘All peptides’ in FIGS. 3A and 3B indicates the number of peptides identified with a 5% FDR, while ‘MHC I peptides’ indicates the number of peptides identified with the corresponding peptide score, 8-11 amino acids in length, and a rank eluted ligand threshold≤2% using netpanMHC4.1b predictions. FIG. 3C: Bar chart indicating the proportion of unique MAPs predicted to bind to a given HLA allele in each sample, using the eluted rank prediction. FIG. 3D: GO term analysis of MAP source genes for CRC-derived cell lines and primary tissues. For tissues, only source genes shared by 4 or more tissues were included in this analysis. FIG. 3E Left panel: Stacked bar chart displaying the proportion of MAPs in each tissue sample derived from protein-coding, hypervariable gene (immunoglobulin or TCR), or non-coding transcripts, or those from unannotated transcripts. Right panel: stacked bar chart displaying the proportion of non-coding MAPs derived from processed transcripts, retained introns, nonstop decay products, nonsense mediated decay products, lncRNA, or those that have no annotated transcript.

FIGS. 4A-E show that novel TSAs identified in CRC derive primarily from non-coding regions, while the majority of TAAs derive from exons. FIG. 4A: Bar chart displaying the number of TSAs identified per sample, with the average number of TSAs per tissue sample indicated with a horizontal line. FIG. 4B: Stacked pie chart identifying the genomic origin of TSAs in the inner pie, as well as what proportion of TSAs are mutated (mTSAs) or aberrantly expressed (aeTSAs). The outer pie demonstrates what proportion of TSAs are from coding or non-coding sequences. FIG. 4C: Bar chart displaying the number of TAAs identified per sample. FIG. 4D: Stacked pie chart identifying the genomic origin of TAAs in the inner pie, and what proportion of TAAs are canonical or non-canonical. The outer pie displays what proportion of TAAs are from coding or non-coding sequences. FIG. 4E: Heatmap displaying the presence or absence of putative TSAs and TAAs in 2 previous publications on CRC immunopeptidomics (8, 15), as well as IEDB and HLA Ligand Atlas (all tissues, and only colon tissue).

FIGS. 5A-B show the RNA expression profiles of putative TSAs and TAAs. FIG. 5A: MA plots displaying the log 2FC of transcripts, in TPM, in CRC compared to the matched NAT on the y-axis and the mean average expression in a given tissue sample (mean of CRC and NAT). Highlighted points indicate the source transcripts of putative TAA and TSAs. Both S4 and S5 plots have a canonical TAA point that is not visible, as it overlaps with another canonical TAA source transcript. FIG. 5B: Heatmap of mean RNA expression in log (rphm+1) of aeTSA coding sequences and TAA coding sequences (divided as canonical TAAs (canTAA) and non-canonical TAAs (non-canTAA) in normal tissues from Genotype Tissue Expression (GTEx) Portal and in pooled TEC samples. MHClow tissues include those from brain, nerve, and testis, which have been shown to lowly express MHC I. A black outline indicates a mean RNA expression >8.55 rphm.

FIGS. 6A-C show the validation of TSA and TAA candidates. FIG. 6A: Heatmap displaying mean RNA expression in log (rphm+1) of TSAs and TAAs in 151 TCGA COAD samples. The proportion of TCGA COAD samples expressing the TSA and TAA sequences at least 10-fold higher than the log-transformed (log (rphm+1)) mean expression of pooled GTEx and mTEC samples is displayed on the left. FIG. 6B: rEpitope immunogenicity scores of various groupings of validated TSAs and TAAs compared to presumably non-immunogenic thymic peptides reported in Adamopoulou et al. 2013. rEpitope suggested threshold of immunogenicity for MHC I peptides (0.36) is indicated by the dashed line. FIG. 6C: Predicted prevalence of tumor antigen-binding MHC class I alleles in US population (IEDB).

FIG. 7 shows an upset plot displaying the number of HLA alleles unique to a given intersection of samples, specifically MAPs that are unique to a given sample or that are uniquely shared by 2 samples.

FIGS. 8A-D show the transcriptomic profile of CRC-derived cell lines, ssGSEA analysis of immune infiltration in CRC tissues, and mutation profile of all samples. FIG. 8A: Principal component analysis (PCA) of the top 500 varying genes of CRC-derived cell line and one normal intestinal cell line (HIEC-6) following paired-end RNA seq and gene readcount normalization with DESeq2. Known MSI cell lines are encircled. FIG. 8B: ssGSEA analysis of immune infiltration in tumor and matched NAT using genes described in Danaher et al. 2017 and GSVA R program (https://github.com/rcastelo/GSVA). FIG. 8C: Scatterplots displaying the SNV counts and INDEL counts of MSS and MSI CRC-derived cell lines determined by SNPEff genomic annotation, with mean and standard error bars. FIG. 8D: Scatterplots displaying the SNV counts and INDEL counts of all MSS and MSI samples (cell lines and tissues), determined by SNPEff genomic annotation, with mean and standard error bars. The difference in the number of INDEL mutations between MSS and MSI samples is statistically significant (p=0.00235).

FIGS. 9A and B show the results of GO term analysis of MSI and MSS primary tissue samples. FIG. 9A: GO term analysis of genes up/downregulated in MSI tumors compared to their adjacent NAT. Genes used for GO term analysis were those with |log 2FC|>1 when compared to their respective NAT using TPM normalized values and that were found to be uniquely differentially expressed in both of the MSI tumor samples (i.e., genes that were only up/downregulated in both of the MSI tumors but not any of the MSS tumors). FIG. 9B: GO term analysis of genes up/downregulated in MSS tumors compared to their NAT. Genes used for GO term analysis were those with |log 2FC|>1 when compared to their respective NAT using TPM normalized values and that were found to be uniquely differentially expressed in 3 or more of the MSI tumor samples (i.e., genes that were up/downregulated in at least 3 MSS tumors but neither of the MSI tumors).

FIGS. 10A-D provide an overview of unique and shared MAPs in CRC-derived cell line and CRC/NAT tissue samples. FIG. 10A: Venn diagram displaying the overlap of MAPs in the MHC I immunopeptidomes of four CRC-derived cell lines. FIG. 10B: Venn diagram displaying the overlap of MAPs in the MHC I immunopeptidomes of six primary tissue samples. FIG. 10C: UpsetR plot displaying the number of MAPs unique to a given intersection of samples, specifically MAPs that are unique to a given sample or that are uniquely shared by two samples. FIG. 10D: Heatmap demonstrating the shared number of MAPs between any two cell line or tissue samples.

FIGS. 11A-E provide an overview of unique and shared MAP source genes in CRC-derived cell line and CRC/NAT tissue samples. FIG. 11A: Top panel: Bar chart displaying the number of unique source genes identified per sample. Bottom panel: Scatterplot indicating the correlation between the number of unique MAPs identified in each sample and the corresponding number of unique source genes (Pearson's r=0.99). Source genes were identified for peptides from coding sequences; any peptide that mapped to more than one source gene was excluded. FIG. 11B: UpsetR plot displaying the number of source genes unique to a given sample or intersection, specifically source genes that are unique to a given sample or that are uniquely shared by two samples. FIG. 11C: Venn diagram displaying the overlap of source genes in the MHC I immunopeptidomes of four CRC-derived cell lines. FIG. 11D: Venn diagram displaying the overlap of source genes in the MHC I immunopeptidomes of six primary tissue samples. FIG. 11E: Heatmap demonstrating the shared number of source genes between any two cell lines or tissue samples.

FIG. 12 is a scatterplot indicating the correlation between the number of unique MAPS identified in each sample and the number of TSAs identified and validated (Pearson's r=0.76).

FIG. 13 is a graph depicting the immunogenicity score of TSAs and TAAs described herein. rEpitope immunogenicity scores of various groupings of validated TSAs and all TAAs compared to presumably non-immunogenic thymic peptides reported in Adamopoulou et al. 2013. rEpitope suggested threshold of immunogenicity for MHC I peptides (0.36) is indicated by the dashed line. This figure differs from FIG. 6A in that it includes all TAAs reported in this work, not only the nine that were chosen for validation.

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

In the studies described herein, the present inventors have identified TSA and TAA candidates from CRC cell lines and CRC specimens using a proteogenomic-based approach. A large fraction of the TSAs derived from aberrantly expressed unmutated genomic sequences which are not expressed in normal tissues, such as non-exonic sequences (e.g., intronic and intergenic sequences). The novel CRC TSA and TAA candidates identified herein may be useful, e.g., for CRC T-cell based immunotherapy and vaccines.

Accordingly, in an aspect, the present disclosure relates to a tumor antigen peptide (TAP) (or tumor-specific peptide), and more particularly an isolated TAP, such as a CRC TAP, comprising, or consisting of, one of the following amino acid sequences:

| Sequence | SEQ ID NO: |
|---|---|
| RMLLSHTGK | 1 |
| LPHRALSGI | 2 |
| GTNPTAAVK | 3 |
| LRHKLVLNR | 4 |
| RIGGVGVEK | 5 |
| SIIETVNSL | 6 |
| TVNTQQYNTK | 7 |
| SVSHLHIFF | 8 |
| TTLENLPQK | 9 |
| AQKLQVRI | 10 |
| GQIELSIYR | 11 |
| HGALSIRSI | 12 |
| RLMKFLPV | 13 |
| SLYISEERK | 14 |
| VQTAVLNV | 15 |
| KIGEVIVTK | 16 |
| TRSTIILHL | 17 |
| SILRGSLGK | 18 |
| RLVSNRVVR | 19 |
| SLKNLEPIK | 20 |
| RSAGPRAPL | 21 |
| VLYRSVLLLK | 22 |
| KAAGAGAAK | 23 |

In another aspect, the present disclosure further relates to the use of a TAP (e.g., isolated TAP) comprising, or consisting of, one of the amino acid sequences below, for the treatment of cancer, and more particularly CRC:

| Sequence | SEQ ID NO: |
|---|---|
| RMLLSHTGK | 1 |
| LPHRALSGI | 2 |
| GTNPTAAVK | 3 |
| LRHKLVLNR | 4 |
| RIGGVGVEK | 5 |
| SIIETVNSL | 6 |
| TVNTQQYNTK | 7 |
| SVSHLHIFF | 8 |
| TTLENLPQK | 9 |
| AQKLQVRI | 10 |
| GQIELSIYR | 11 |
| HGALSIRSI | 12 |
| RLMKFLPV | 13 |
| SLYISEERK | 14 |
| VQTAVLNV | 15 |
| KIGEVIVTK | 16 |
| TRSTIILHL | 17 |
| SILRGSLGK | 18 |
| RLVSNRVVR | 19 |
| SLKNLEPIK | 20 |
| RSAGPRAPL | 21 |
| VLYRSVLLLK | 22 |
| KAAGAGAAK | 23 |
| VEAPHLPSF | 25 |
| EYNSDLHQF | 26 |
| TYKYVDINTF | 27 |
| VIDEPVRL | 28 |
| RYLEKFYGL | 29 |
| KRLPSPSLF | 30 |
| KSINEFWNK | 31 |
| RVFHGNETTK | 32 |
| RIQLPVVSK | 33 |
| SIFRTPISK | 34 |
| AEYHSQSRA | 35 |
| IHNSDQILL | 36 |
| KMHQSRISY | 37 |
| QMAGLRDTY | 38 |
| RIKNWGSGMTF | 39 |
| AQYDQASTKY | 40 |
| FVDNQYWRY | 41 |
| QLDSAVKNLY | 42 |
| EQLKVTTI | 43 |
| FRSWAVQTF | 44 |
| KLYQSIIGY | 45 |
| RVAKTNSLR | 46 |
| SANVSKVSF | 47 |
| SQHDILQMI | 48 |
| VAKTPRNTY | 49 |
| RPRLSAILF | 50 |

In an embodiment, the TAP comprises, or consists of, one of the following amino acid sequences: SEQ ID NOs: 1-23. In an embodiment, the TAP comprises, or consists of, one of the following amino acid sequences: SEQ ID NOs: 1-17, 22, 25, 27, 29, 31, 33, 38, 40, 41 and 46. In a further embodiment, the TAP comprises, or consists of, one of the following amino acid sequences: SEQ ID NOs: 1-17, 22, and 25. In a further embodiment, the TAP comprises, or consists of, one of the following amino acid sequences: SEQ ID NOs: 1-17 and 22.

In general, peptides such as tumor antigen peptides (TAPs) presented in the context of HLA class I vary in length from about 7 or 8 to about 15, or preferably 8 to 14 amino acid residues. In some embodiments of the methods of the disclosure, longer peptides comprising the TAP sequences defined herein are artificially loaded into cells such as antigen presenting cells (APCs), processed by the cells and the TAP is presented by MHC class I molecules at the surface of the APC. In this method, peptides/polypeptides longer than 15 amino acid residues can be loaded into APCs, are processed by proteases in the APC cytosol providing the corresponding TAP as defined herein for presentation. In some embodiments, the precursor peptide/polypeptide that is used to generate the TAP defined herein is for example 1000, 500, 400, 300, 200, 150, 100, 75, 50, 45, 40, 35, 30, 25, 20 or 15 amino acids or less. Thus, all the methods and processes using the TAPs described herein include the use of longer peptides or polypeptides (including the native protein), i.e., tumor antigen precursor peptides/polypeptides, to induce the presentation of the “final” 8-14 TAP following processing by the cell (APCs). In some embodiments, the herein-mentioned TAP is about 8 to 14, 8 to 13, or 8 to 12 amino acids long (e.g., 8, 9, 10, 11, 12 or 13 amino acids long), small enough for a direct fit in an HLA class I molecule. In an embodiment, the TAP comprises 20 amino acids or less, preferably 15 amino acids or less, more preferably 14 amino acids or less. In an embodiment, the TAP comprises at least 7 amino acids, preferably at least 8 amino acids or less, more preferably at least 9 amino acids.

The term “amino acid” as used herein includes both L- and D-isomers of the naturally occurring amino acids as well as other amino acids (e.g., naturally-occurring amino acids, non-naturally-occurring amino acids, amino acids which are not encoded by nucleic acid sequences, etc.) used in peptide chemistry to prepare synthetic analogs of TAPs. Examples of naturally occurring amino acids are glycine, alanine, valine, leucine, isoleucine, serine, threonine, etc. Other amino acids include for example non-genetically encoded forms of amino acids, as well as a conservative substitution of an L-amino acid. Naturally-occurring non-genetically encoded amino acids include, for example, beta-alanine, 3-amino-propionic acid, 2,3-diaminopropionic acid, alpha-aminoisobutyric acid (Aib), 4-amino-butyric acid, N-methylglycine (sarcosine), hydroxyproline, ornithine (e.g., L-ornithine), citrulline, t-butylalanine, t-butylglycine, N-methylisoleucine, phenylglycine, cyclohexylalanine, norleucine (Nle), norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, L-homoarginine (Hoarg), N-acetyl lysine, 2-amino butyric acid, 2-amino butyric acid, 2,4,-diaminobutyric acid (D- or L-), p-aminophenylalanine, N-methylvaline, homocysteine, homoserine (HoSer), cysteic acid, epsilon-amino hexanoic acid, delta-amino valeric acid, or 2,3-diaminobutyric acid (D- or L-), etc. These amino acids are well known in the art of biochemistry/peptide chemistry. In an embodiment, the TAP comprises only naturally-occurring amino acids.

In embodiments, the TAPs described herein include peptides with altered sequences containing substitutions of functionally equivalent amino acid residues, relative to the herein-mentioned sequences. For example, one or more amino acid residues within the sequence can be substituted by another amino acid of a similar polarity (having similar physico-chemical properties) which acts as a functional equivalent, resulting in a silent alteration. Substitution for an amino acid within the sequence may be selected from other members of the class to which the amino acid belongs. For example, positively charged (basic) amino acids include arginine, lysine and histidine (as well as homoarginine and ornithine). Nonpolar (hydrophobic) amino acids include leucine, isoleucine, alanine, phenylalanine, valine, proline, tryptophan and methionine. Uncharged polar amino acids include serine, threonine, cysteine, tyrosine, asparagine and glutamine. Negatively charged (acidic) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions. The herein-mentioned TAP may comprise all L-amino acids, all D-amino acids or a mixture of L- and D-amino acids. In an embodiment, the herein-mentioned TAP comprises all L-amino acids.

In an embodiment, in the sequences of the TAPs comprising or consisting of one of sequences of SEQ ID NOs: 1-23 and 25-50, the amino acid residues that do not substantially contribute to interactions with the T-cell receptor may be modified by replacement with other amino acid whose incorporation does not substantially affect T-cell reactivity and does not eliminate binding to the relevant MHC.

The TAP may also be N- and/or C-terminally capped or modified to prevent degradation, increase stability, affinity and/or uptake. Thus, in another aspect, the present disclosure provides a modified TAP of the formula Z¹—X—Z², wherein X is a TAP comprising, or consisting of, one of the amino acid sequences of SEQ ID NOs: 1-23 and 25-50.

In an embodiment, the amino terminal residue (i.e., the free amino group at the N-terminal end) of the TAP is modified (e.g., for protection against degradation), for example by covalent attachment of a moiety/chemical group (Z¹). Z¹ may be a straight chained or branched alkyl group of one to eight carbons, or an acyl group (R—CO—), wherein R is a hydrophobic moiety (e.g., acetyl, propionyl, butanyl, iso-propionyl, or iso-butanyl), or an aroyl group (Ar—CO—), wherein Ar is an aryl group. In an embodiment, the acyl group is a C₁-C₁₆ or C₃-C₁₆ acyl group (linear or branched, saturated or unsaturated), in a further embodiment, a saturated C₁-C₆ acyl group (linear or branched) or an unsaturated C₃-C₆ acyl group (linear or branched), for example an acetyl group (CH₃—CO—, Ac). In an embodiment, Z¹ is absent. The carboxy terminal residue (i.e., the free carboxy group at the C-terminal end of the TAP) of the TAP may be modified (e.g., for protection against degradation), for example by amidation (replacement of the OH group by a NH₂ group), thus in such a case Z² is a NH₂ group. In an embodiment, Z² may be an hydroxamate group, a nitrile group, an amide (primary, secondary or tertiary) group, an aliphatic amine of one to ten carbons such as methyl amine, iso-butylamine, iso-valerylamine or cyclohexylamine, an aromatic or arylalkyl amine such as aniline, napthylamine, benzylamine, cinnamylamine, or phenylethylamine, an alcohol or CH₂OH. In an embodiment, Z² is absent. In an embodiment, the TAP comprises one of the amino acid sequences of SEQ ID NOs: 1-24 and 26-50. In an embodiment, the TAP consists of one of the amino acid sequences of SEQ ID NOs: 1-24 and 26-50, i.e., wherein Z¹ and Z² are absent.

In another aspect, the present disclosure provides a TAP binding to an HLA-A*02:01 molecule, comprising or consisting of the sequence of SEQ ID NO:6. Because HLA alleles show promiscuity (certain HLA alleles present similar epitopes), the above-identified TAP may further bind to HLA-A*02:05, HLA-A*02:06 and/or HLA-A*02:07 molecules.

In another aspect, the present disclosure provides a TAP binding to an HLA-A*03:01 molecule, comprising or consisting of the sequence of SEQ ID NO:1, 11, 14, 38, 45 or 46, preferably SEQ ID NO:1, 11, or 14. Because HLA alleles show promiscuity (certain HLA alleles present similar epitopes), the above-identified TAP may further bind to HLA-A*03:02, HLA-A*11:01 or HLA-A*30:01 molecules.

In another aspect, the present disclosure provides a TAP binding to an HLA-A*03:02 molecule, comprising or consisting of the sequence of SEQ ID NOs: 3, 5, 7, 16, 23, 31, 32, 33 or 34, preferably SEQ ID NOs: 3, 5, 7, 16 or 23. Because HLA alleles show promiscuity (certain HLA alleles present similar epitopes), the above-identified TAP may further bind to HLA-A*03:01, HLA-A*11:01 or HLA-A*30:01 molecules

In another aspect, the present disclosure provides a TAP binding to an HLA-A*11:01 molecule, comprising or consisting of the sequence of SEQ ID NO:9, 18, 33 or 34, preferably SEQ ID NO:9 or 18. Because HLA alleles show promiscuity (certain HLA alleles present similar epitopes), the above-identified TAP may further bind to HLA-A*03:01, HLA-A*03:02, HLA-A*31:01 and/or HLA-A*68:01 molecules.

In another aspect, the present disclosure provides a TAP binding to an HLA-A*23:01 molecule, comprising or consisting of the sequence of SEQ ID NO:29. Because HLA alleles show promiscuity (certain HLA alleles present similar epitopes), the above-identified TAP may further bind to HLA-A*24:02 molecules.

In another aspect, the present disclosure provides a TAP binding to an HLA-A*24:02 molecule, comprising or consisting of the sequence of SEQ ID NO:26, 27 or 29. Because HLA alleles show promiscuity (certain HLA alleles present similar epitopes), the above-identified TAP may further bind to HLA-A*23:01 molecules.

In another aspect, the present disclosure provides a TAP binding to an HLA-A*30:01 molecule, comprising or consisting of the sequence of SEQ ID NO: 19, 20 or 23. Because HLA alleles show promiscuity (certain HLA alleles present similar epitopes), the above-identified TAP may further bind to HLA-A*30:02 and/or HLA-B*15:02 molecules.

In another aspect, the present disclosure provides a TAP binding to an HLA-A*32:01 molecule, comprising or consisting of the sequence of SEQ ID NO: 8, 37 or 39, preferably SEQ ID NO: 8. Because HLA alleles show promiscuity (certain HLA alleles present similar epitopes), the above-identified TAP may further bind to HLA-B*57:01 and/or HLA-B*58:01 molecules.

In another aspect, the present disclosure provides a TAP binding to an HLA-B*07:02 molecule, comprising or consisting of the sequence of SEQ ID NO:2, 21, 24 or 50, preferably SEQ ID NO:2 or 21. Because HLA alleles show promiscuity (certain HLA alleles present similar epitopes), the above-identified TAP may further bind to HLA-B*35:02, HLA-B*35:03, HLA-B*55:01 and/or HLA-B*56:01 molecules.

In another aspect, the present disclosure provides a TAP binding to an HLA-B*13:02 molecule, comprising or consisting of the sequence of SEQ ID NO: 13 or 48, preferably SEQ ID NO: 13.

In another aspect, the present disclosure provides a TAP binding to an HLA-B*18:01 molecule, comprising or consisting of the sequence of SEQ ID NO: 25. Because HLA alleles show promiscuity (certain HLA alleles present similar epitopes), the above-identified TAP may further bind to HLA-B*40:01, HLA-B*44:02, HLA-B*44:03 and/or HLA-B*45:01 molecules.

In another aspect, the present disclosure provides a TAP binding to an HLA-B*27:05 molecule, comprising or consisting of the sequence of SEQ ID NO: 4. Because HLA alleles show promiscuity (certain HLA alleles present similar epitopes), the above-identified TAP may further bind to HLA-B*27:02

In another aspect, the present disclosure provides a TAP binding to an HLA-B*52:01 molecule, comprising or consisting of the sequence of SEQ ID NO: 10, 12, 15 or 43, preferably SEQ ID NO: 10, 12, or 15. Because HLA alleles show promiscuity (certain HLA alleles present similar epitopes), the above-identified TAP may further bind to HLA-B*51:01.

In another aspect, the present disclosure provides a TAP binding to an HLA-C*06:02 molecule, comprising or consisting of the sequence of SEQ ID NO:17 or 44, preferably SEQ ID NO: 17. Because HLA alleles show promiscuity (certain HLA alleles present similar epitopes), the above-identified TAP may further bind to HLA-B*27:02, HLA-C*07:01 and/or HLA-C*07:02 molecules.

In another aspect, the present disclosure provides a TAP binding to an HLA-C*12:02 molecule, comprising or consisting of the sequence of SEQ ID NO: 47 or 49. Because HLA alleles show promiscuity (certain HLA alleles present similar epitopes), the above-identified TAP may further bind to HLA-B*46:01, HLA-C*03:02, HLA-C*03:03, HLA-C*03:04, HLA-C*08:01, HLA-C*12:03, HLA-C*15:02 and/or HLA-C*16:01 molecules.

In an embodiment, the TAP is encoded by a sequence located a non-protein coding region of the genome. In an embodiment, the TAP is encoded by a sequence located in an untranslated transcribed region (UTR), i.e., a 3′-UTR or 5′-UTR region. In another embodiment, the TAP is encoded by a sequence located in an intron. In another embodiment, the TAP is encoded by a sequence located in an intergenic region. In another embodiment, the TAP is encoded by a sequence located in an exon and originates from a frameshift.

The TAPs of the disclosure may be produced by expression in a host cell comprising a nucleic acid encoding the TAPs (recombinant expression) or by chemical synthesis (e.g., solid-phase peptide synthesis). Peptides can be readily synthesized by manual and/or automated solid phase procedures well known in the art. Suitable syntheses can be performed for example by utilizing “T-boc” or “Fmoc” procedures. Techniques and procedures for solid phase synthesis are described in for example Solid Phase Peptide Synthesis: A Practical Approach, by E. Atherton and R. C. Sheppard, published by IRL, Oxford University Press, 1989. Alternatively, the MiHA peptides may be prepared by way of segment condensation, as described, for example, in Liu et al., Tetrahedron Lett. 37:933-936, 1996; Baca et al., J. Am. Chem. Soc. 117:1881-1887, 1995; Tam et al., Int. J. Peptide Protein Res. 45:209-216, 1995; Schnolzer and Kent, Science 256:221-225, 1992; Liu and Tam, J. Am. Chem. Soc. 116:4149-4153, 1994; Liu and Tam, Proc. Natl. Acad. Sci. USA 91:6584-6588, 1994; and Yamashiro and Li, Int. J. Peptide Protein Res. 31:322-334, 1988). Other methods useful for synthesizing the TAPs are described in Nakagawa et al., J. Am. Chem. Soc. 107:7087-7092, 1985. In an embodiment, the TAP is chemically synthesized (synthetic peptide). Another embodiment of the present disclosure relates to a non-naturally occurring peptide wherein said peptide consists or consists essentially of an amino acid sequences defined herein and has been synthetically produced (e.g., synthesized) as a pharmaceutically acceptable salt. The salts of the TAPs according to the present disclosure differ substantially from the peptides in their state(s) in vivo, as the peptides as generated in vivo are no salts. The non-natural salt form of the peptide may modulate the solubility of the peptide, in particular in the context of pharmaceutical compositions comprising the peptides, e.g., the peptide vaccines as disclosed herein. Preferably, the salts are pharmaceutically acceptable salts of the peptides.

In an embodiment, the herein-mentioned TAP is isolated or substantially pure. A compound such as a peptide or nucleic acid is “isolated” or “substantially pure” when it is separated from the components that are present in the natural environment of the molecule or a naturally occurring source macromolecule (e.g., including other nucleic acids, proteins, lipids, sugars, etc.). Typically, a compound is substantially pure when it is at least 60%, more generally 75%, 80% or 85%, preferably over 90% and more preferably over 95%, by weight, of the total material in a sample. Thus, for example, a polypeptide that is chemically synthesized or produced by recombinant technology will generally be substantially free from its naturally associated components, e.g., components of its source macromolecule. A nucleic acid molecule is substantially pure when it is not immediately contiguous with (i.e., covalently linked to) the coding sequences with which it is normally contiguous in the naturally occurring genome of the organism from which the nucleic acid is derived. A substantially pure compound can be obtained, for example, by extraction from a natural source; by expression of a recombinant nucleic acid molecule encoding a peptide compound; or by chemical synthesis. Purity can be measured using any appropriate method such as column chromatography, gel electrophoresis, HPLC, etc. In an embodiment, the TAP is in solution. In another embodiment, the TAP is in solid form, e.g., lyophilized.

In an embodiment, the TAP is encoded by a sequence located a non-protein coding region of the genome. In an embodiment, the TAP is encoded by a sequence located in an intergenic region. In another embodiment, the TAP is encoded by a non-coding RNA (ncRNA).

In another aspect, the disclosure further provides a synthetic long peptide (SLP) comprising at least one of the TAP described herein. In an embodiment, the SLP comprises at least two TAPs, wherein at least one of the TAP is a TAP as described herein. In an embodiment, the SLP comprises at least two, three, four or five of the TAPs described herein. In an embodiment, the SLP comprises at least one of the TAPs described herein linked to one or more amino acid sequences or domains that confer desired properties to the SLP, such as sequences or domains that stabilize the SLP and/or that improve processing and presentation by MHC molecules, for example a sequence comprising a motif cleavable by cellular proteases such as cathepsins. In another embodiment, the SLP comprises at least one of the TAPs described herein, and a TAP that binds to MHC class II molecules. The TAPs may directly attached to each other, or may be indirectly attached via a linker such as a short amino acid linker. In embodiments, the linker comprises about 4 to about 20 amino acids, or about 4 to about 15 amino acids, e.g., 4, 5, 6, 7, 8, 9, 10, 11, 12, 13, 14 or 15 amino acids. In an embodiment, the linker comprises glycine residues, serine residues, proline residues, threonine residues, or a mixture thereof. In an embodiment, the SLP has a length of 200, 150, 100, 90, 80, 70, 60 or 50 amino acids or less. In a further embodiment, the SLP has a length of 20 to 50, 45 or 40 amino acids, for example from 20 or 25 amino acids to 30, 35 or 40 amino acids. “Synthetic”, as used herein, refers to a peptide or nucleic molecule that is not isolated from its natural sources, e.g., which is produced through recombinant technology or using chemical synthesis.

In another aspect, the disclosure further provides a nucleic acid (e.g., isolated) encoding the herein-mentioned TAPs or a tumor antigen precursor-peptide or SLP. In an embodiment, the nucleic acid comprises from about 21 nucleotides to about 45 nucleotides, from about 24 to about 45 nucleotides, for example 24, 27, 30, 33, 36, 39, 42 or 45 nucleotides.

In an embodiment, the nucleic acid (DNA, RNA) encoding the TAP of the disclosure comprises any one of the sequences defined in SEQ ID NOs: 51-73 and 75-100, or SEQ ID NOS: 51-67, 72, 75, 77, 79, 81, 83, 88, 90, 91 and 96, or SEQ ID NOs: 51-67, 72 and 75 (e.g., SEQ ID NOs: 51-67 and 72), or a corresponding RNA sequence (i.e., in which the thymine nucleobases (T) are replaced by uracil nucleobases (U)). In an embodiment, the nucleic acid encoding the TAP is an mRNA molecule. In an embodiment, the nucleic acid is in solution. In another embodiment, the nucleic acid is in solid form, e.g., lyophilized.

A nucleic acid of the disclosure may be used for recombinant expression of the TAP or SLP of the disclosure, and may be included in a vector or plasmid, such as a cloning vector or an expression vector, which may be transfected into a host cell. In an embodiment, the disclosure provides a cloning, expression or viral vector or plasmid comprising a nucleic acid sequence encoding the TAP of the disclosure. Alternatively, a nucleic acid encoding a TAP of the disclosure may be incorporated into the genome of the host cell. In either case, the host cell expresses the TAP or protein encoded by the nucleic acid. The term “host cell” as used herein refers not only to the particular subject cell, but to the progeny or potential progeny of such a cell. A host cell can be any prokaryotic (e.g., E. coli) or eukaryotic cell (e.g., insect cells, yeast cells, plant cells, or mammalian cells) capable of expressing the TAPs described herein. The vector or plasmid contains the necessary elements for the transcription and translation of the inserted coding sequence, and may contain other components such as resistance genes, cloning sites, etc. Methods that are well known to those skilled in the art may be used to construct expression vectors containing sequences encoding peptides or polypeptides and appropriate transcriptional and translational control/regulatory elements operably linked thereto. These methods include in vitro recombinant DNA techniques, synthetic techniques, and in vivo genetic recombination. Such techniques are described in Sambrook. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y. “Operably linked” refers to a juxtaposition of components, particularly nucleotide sequences, such that the normal function of the components can be performed. Thus, a coding sequence that is operably linked to regulatory sequences refers to a configuration of nucleotide sequences wherein the coding sequences can be expressed under the regulatory control, that is, transcriptional and/or translational control, of the regulatory sequences. “Regulatory/control region” or “regulatory/control sequence”, as used herein, refers to the non-coding nucleotide sequences that are involved in the regulation of the expression of a coding nucleic acid. Thus, the term regulatory region includes promoter sequences, regulatory protein binding sites, upstream activator sequences, and the like. The vector (e.g., expression vector) may have the necessary 5′ upstream and 3′ downstream regulatory elements such as promoter sequences such as CMV, PGK and EF-1a promoters, ribosome recognition and binding TATA box, and 3′ UTR AAUAAA transcription termination sequence for the efficient gene transcription and translation in its respective host cell. Other suitable promoters include the constitutive promoter of simian vims 40 (SV40) early promoter, mouse mammary tumor virus (MMTV), HIV LTR promoter, MoMuLV promoter, avian leukemia virus promoter, EBV immediate early promoter, and Rous sarcoma vims promoter. Human gene promoters may also be used, including, but not limited to the actin promoter, the myosin promoter, the hemoglobin promoter, and the creatine kinase promoter. In certain embodiments inducible promoters are also contemplated as part of the vectors expressing the TAP. This provides a molecular switch capable of turning on expression of the polynucleotide sequence of interest or turning off expression. Examples of inducible promoters include, but are not limited to a metallothionine promoter, a glucocorticoid promoter, a progesterone promoter, or a tetracycline promoter. Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are Lenti-X™ Bicistronic Expression System (Neo) vectors (Contech), pClneo vectors (Promega) for expression in mammalian cells; pLenti4/V5-DEST™, pLenti6/V5-DEST™, and pLenti6.2N5-GW/lacZ (Invitrogen) for lentivirus-mediated gene transfer and expression in mammalian cells. The coding sequences of the TAPs disclosed herein can be ligated into such expression vectors for the expression of the TAP in mammalian cells.

In certain embodiments, the nucleic acids encoding the TAP of the present disclosure are provided in a viral vector. A viral vector can be those derived from adenovirus, vaccinia virus, retrovirus, lentivirus, or foamy virus. As used herein, the term “viral vector” refers to a nucleic acid vector construct that includes at least one element of viral origin and has the capacity to be packaged into a viral vector particle. The viral vector can contain the coding sequence for the various proteins described herein in place of nonessential viral genes. In another embodiment, the nucleic acids encoding the TAP of the present disclosure are provided in a self-amplifying or self-replicating RNA (srRNA) vectors. srRNAs are derived from positive-strand RNA viruses where the structural proteins have been removed and replaced with heterologous genes of interest. srRNAs have been successfully derived from flaviviruses, nodamura viruses, nidoviruses, and alphaviruses with therapeutic versions of the technology providing the structural proteins in trans to create single cycle viral replicon particles (VRPs) (see, e.g., Aliahmad et al. Next generation self-replicating RNA vectors for vaccines and immunotherapies. Cancer Gene Ther (2022). https://doi.org/10.1038/s41417-022-00435-8). The vector and/or particle can be utilized for the purpose of transferring DNA, RNA or other nucleic acids into cells either in vitro or in vivo. Numerous forms of viral vectors are known in the art.

In embodiment, the nucleic acid (DNA, RNA) encoding the TAP of the disclosure is comprised within a vesicle or nanoparticle such as a lipid vesicle (e.g., liposome) or lipid nanoparticle (LNP), or any other suitable vehicle. Thus, in another aspect, the present disclosure provides a vesicle or nanoparticle, such as a lipid vesicle or nanoparticle, comprising a nucleic acid, such as an mRNA, encoding one or more of the TAP described herein.

The term liposome as used herein in accordance with its usual meaning, referring to microscopic lipid vesicles composed of a bilayer of phospholipids or any similar amphipathic lipids (e.g., sphingolipids) encapsulating an internal aqueous medium.

The term “lipid nanoparticle” refers to liposome-like structure that may include one or more lipid bilayer rings surrounding an internal aqueous medium similar to liposomes, or micellar-like structures that encapsulates molecules (e.g., nucleic acids) in a non-aqueous core. Lipid nanoparticles typically contain cationic lipids, such as ionizable cationic lipids. Examples of cationic lipids that may be used for LNPs include DOTMA, DOSPA, DOTAP, ePC, DLin-MC3-DMA, C12-200, ALC-0315, cKK-E12, Lipid H (SM-102), OF-Deg-Lin, A2-Iso5-2DC18, 306Oᵢ₁₀, BAME-O16B, TT3, 9A1P9, FTT5, COATSOME® SS-E, COATSOME® SS-EC, COATSOME® SS-OC and COATSOME® SS-OP (see, e.g., Hou et al., Nature Reviews Materials, volume 6, pages 1078-1094 (2021); Tenchov et al., ACS Nano, 15, 16982-17015 (2021).

Liposomes and lipid nanoparticles typically include other lipid components such as lipids, lipid-like materials, and polymers that can improve liposome or nanoparticle properties, such as stability, delivery efficacy, tolerability and biodistribution. These include phospholipids (e.g., phosphatidylcholines, phosphatidylethanolamines, phosphatidylserines, and phosphatidylglycerol) such as 1,2-distearoyl-sn-glycero-3-phosphocholine (DSPC) and DOPE, sterols (such as cholesterol and cholesterol derivatives), PEGylated lipids (PEG-lipids) such as 1,2-dimyristoyl-rac-glycero-3-methoxypolyethylene glycol-2000 (PEG₂₀₀₀-DMG) and 1,2-distearoyl-rac-glycero-3-methoxypolyethylene glycol-2000 (PEG₂₀₀₀-DSG).

In an embodiment, the lipid nanoparticle according to the present disclosure comprises one or more cationic lipids, such as ionizable cationic lipids. Examples of ionizable cationic lipids include those listed in PCT publications Nos. WO 2017/061150 and WO 2019/188867, which encompassed ionizable cationic lipids commercialized under the tradenames COATSOME® SS-E, COATSOME® SS-EC, COATSOME® SS-OC and COATSOME® SS-OP.

The nucleic acid (e.g., mRNA) encoding one or more of the TAP, may be modified, for example to increase stability and/or reduce immunogenicity. For example, the 5′ end may be capped to stabilize the molecule and decrease immunogenicity (for example, as described in U.S. Pat. Nos. 10,519,189 and 10,494,399). One or more nucleosides of the mRNA may be modified or substituted with 1-methyl pseudo-uridine to either increase stability of the molecule or reduce recognition of the molecule by the innate immune system. A form of modified nucleosides are described in U.S. Pat. No. 9,371,511. Other types of modifications that may be made to the mRNA include incorporation of anti-reverse cap analog (ARCA), 5′-methyl-cytidine triphosphate (m5CTP), N6-methyl-adenosine-5′-triphosphate (m6ATP), 2-thio-uridine triphosphate (s2UTP), pseudouridine triphosphate, N1Methylpseudouridine triphosphate or 5-Methoxyuridine triphosphate (5moUTP). The mRNA may also include additional modifications to the 5′- and/or 3′-untranslated regions (UTRs) and polyadenylation (polyA) tail (see, for example, Kim et al., Molecular & cellular toxicology vol. 18,1 (2022): 1-8). All these modifications and other modifications to the nucleic acid (e.g., mRNA) encoding the TAP are encompassed by the present disclosure.

In another aspect, the present disclosure provides an MHC class I molecule comprising (i.e., presenting or bound to) one or more of the TAP of SEQ ID NOs: SEQ ID NOs: 1-23 and 25-50, preferably SEQ ID NOs: 1-23, such as SEQ ID NOs: 1-17 and 22.

In an embodiment, the MHC class I molecule is an HLA-A2 molecule, in a further embodiment an HLA-A*02:01 molecule. In an embodiment, the MHC class I molecule is an HLA-A3 molecule, in a further embodiment an HLA-A*03:01 or HLA-A*03:02 molecule. In another embodiment, the MHC class I molecule is an HLA-A11 molecule, in a further embodiment an HLA-A*11:01 molecule. In an embodiment, the MHC class I molecule is an HLA-A23 molecule, in a further embodiment an HLA-A*23:01 molecule. In an embodiment, the MHC class I molecule is an HLA-A24 molecule, in a further embodiment an HLA-A*24:02 molecule. In an embodiment, the MHC class I molecule is an HLA-A30 molecule, in a further embodiment an HLA-A*30:01 molecule. In an embodiment, the MHC class I molecule is an HLA-A32 molecule, in a further embodiment an HLA-A*32:01 molecule. In another embodiment, the MHC class I molecule is an HLA-B07 molecule, in a further embodiment an HLA-B*07:02 molecule. In another embodiment, the MHC class I molecule is an HLA-B13 molecule, in a further embodiment an HLA-B*13:02 molecule. In another embodiment, the MHC class I molecule is an HLA-B18 molecule, in a further embodiment an HLA-B*18:01 molecule. In another embodiment, the MHC class I molecule is an HLA-B27 molecule, in a further embodiment an HLA-B*27:05 molecule. In another embodiment, the MHC class I molecule is an HLA-B52 molecule, in a further embodiment an HLA-B*52:01 molecule. In another embodiment, the MHC class I molecule is an HLA-C06 molecule, in a further embodiment an HLA-C*06:02 molecule. In another embodiment, the MHC class I molecule is an HLA-C04 molecule, in a further embodiment an HLA-C*04:01 molecule. In another embodiment, the MHC class I molecule is an HLA-C12 molecule, in a further embodiment an HLA-C*12:02 molecule.

In an embodiment, the TAP (e.g., SEQ ID NOs: SEQ ID NOS: 1-23 and 25-50, preferably SEQ ID NOs: 1-23) is non-covalently bound to the MHC class I molecule (i.e., the TAP is loaded into, or non-covalently bound to the peptide binding groove/pocket of the MHC class I molecule). In another embodiment, the TAP is covalently attached/bound to the MHC class I molecule (alpha chain). In such a construct, the TAP and the MHC class I molecule (alpha chain) are produced as a synthetic fusion protein, typically with a short (e.g., 5 to 20 residues, preferably about 8-12, e.g., 10) flexible linker or spacer (e.g., a polyglycine linker). In another aspect, the disclosure provides a nucleic acid encoding a fusion protein comprising a TAP defined herein fused to an MHC class I molecule (alpha chain). In an embodiment, the MHC class I molecule (alpha chain)-peptide complex is multimerized. Accordingly, in another aspect, the present disclosure provides a multimer of MHC class I molecule loaded (covalently or not) with the herein-mentioned TAP. Such multimers may be attached to a tag, for example a fluorescent tag, which allows the detection of the multimers. A great number of strategies have been developed for the production of MHC multimers, including MHC dimers, tetramers, pentamers, octamers, etc. (reviewed in Bakker and Schumacher, Current Opinion in Immunology 2005, 17:428-433). MHC multimers are useful, for example, for the detection and purification of antigen-specific T cells. Thus, in another aspect, the present disclosure provides a method for detecting or purifying (isolating, enriching) CD8⁺ T lymphocytes specific for a TAP defined herein, the method comprising contacting a cell population with a multimer of MHC class I molecule loaded (covalently or not) with the TAP; and detecting or isolating the CD8⁺ T lymphocytes bound by the MHC class I multimers. CD8⁺ T lymphocytes bound by the MHC class I multimers may be isolated using known methods, for example fluorescence activated cell sorting (FACS) or magnetic activated cell sorting (MACS).

In yet another aspect, the present disclosure provides a cell (e.g., a host cell), in an embodiment an isolated cell, comprising the herein-mentioned nucleic acid, vector or plasmid of the disclosure, i.e., a nucleic acid or vector encoding one or more TAPs. In another aspect, the present disclosure provides a cell expressing at its surface an MHC class I molecule (e.g., an MHC class I molecule of one of the alleles disclosed above) bound to or presenting a TAP according to the disclosure. In one embodiment, the host cell is a eukaryotic cell, such as a mammalian cell, preferably a human cell. a cell line or an immortalized cell. In another embodiment, the cell is an antigen-presenting cell (APC). In one embodiment, the host cell is a primary cell, a cell line or an immortalized cell. In another embodiment, the cell is an antigen-presenting cell (APC). Nucleic acids and vectors can be introduced into cells via conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (supra), and other laboratory manuals. Methods for introducing nucleic acids into mammalian cells in vivo are also known, and may be used to deliver the vector or plasmid of the disclosure to a subject for gene therapy.

Cells such as APCs can be loaded with one or more TAPs using a variety of methods known in the art. As used herein “loading a cell” with a TAP means that RNA or DNA encoding the TAP, or the TAP, is transfected into the cells or alternatively that the APC is transformed with a nucleic acid encoding the TAP. The cell can also be loaded by contacting the cell with exogenous TAPs that can bind directly to MHC class I molecule present at the cell surface (e.g., peptide-pulsed cells). The TAPs may also be fused to a domain or motif that facilitates its presentation by MHC class I molecules, for example to an endoplasmic reticulum (ER) retrieval signal, a C-terminal Lys-Asp-Glu-Leu sequence (see Wang et al., Eur J Immunol. 2004 December; 34 (12): 3582-94).

In another aspect, the present disclosure provides a composition or peptide combination/pool comprising any one of, or any combination of, the TAPs defined herein (or a nucleic acid encoding said peptide(s)). In an embodiment, the composition comprises any combination of the TAPs defined herein (any combination of 2, 3, 4, 5, 6, 7, 8, 9, 10 or more TAPs), or a combination of nucleic acids encoding said TAPs). Compositions comprising any combination/sub-combination of the TAPs defined herein are encompassed by the present disclosure. In another embodiment, the combination or pool may comprise one or more known tumor antigens.

Thus, in another aspect, the present disclosure provides a composition comprising any one of, or any combination of, the TAPs or SLP(s) defined herein (e.g., comprising or consisting of the sequence of SEQ ID NOs: 1-23 and 25-50, preferably SEQ ID NOs: 1-23, such as SEQ ID NOs: 1-17, and 22) and a cell expressing an MHC class I molecule (e.g., an MHC class I molecule of one of the alleles disclosed above). APC for use in the present disclosure are not limited to a particular type of cell and include professional APCs such as dendritic cells (DCs), Langerhans cells, macrophages and B cells, which are known to present proteinaceous antigens on their cell surface so as to be recognized by CD8⁺ T lymphocytes. For example, an APC can be obtained by inducing DCs from peripheral blood monocytes and then contacting (stimulating) the TAPs, either in vitro, ex vivo or in vivo. APC can also be activated to present a TAP in vivo where one or more of the TAPs of the disclosure are administered to a subject and APCs that present a TAP are induced in the body of the subject. The phrase “inducing an APC” or “stimulating an APC” includes contacting or loading a cell with one or more TAPs, or nucleic acids encoding the TAPs such that the TAPs are presented at its surface by MHC class I molecules. As noted herein, according to the present disclosure, the TAPs may be loaded indirectly for example using longer peptides/polypeptides comprising the sequence of the TAPs (including the native protein), which is then processed (e.g., by proteases) inside the APCs to generate the TAP/MHC class I complexes at the surface of the cells. After loading APCs with TAPs and allowing the APCs to present the TAPs, the APCs can be administered to a subject as a vaccine. For example, the ex vivo administration can include the steps of: (a) collecting APCs from a first subject, (b) contacting/loading the APCs of step (a) with a TAP to form MHC class I/TAP complexes at the surface of the APCs; and (c) administering the peptide-loaded APCs to a second subject in need for treatment.

The first subject and the second subject may be the same subject (e.g., autologous vaccine), or may be different subjects (e.g., allogeneic vaccine). Alternatively, according to the present disclosure, use of a TAP described herein (or a combination thereof) for manufacturing a composition (e.g., a pharmaceutical composition) for inducing antigen-presenting cells is provided. In addition, the present disclosure provides a method or process for manufacturing a pharmaceutical composition for inducing antigen-presenting cells, wherein the method or the process includes the step of admixing or formulating the TAP, or a combination thereof, with a pharmaceutically acceptable carrier. Cells such as APCs expressing an MHC class I molecule (e.g., any of the above-noted HLA molecules) loaded with any one of, or any combination of, the TAPs defined herein, may be used for stimulating/amplifying CD8⁺ T lymphocytes, for example autologous CD8⁺ T lymphocytes. Accordingly, in another aspect, the present disclosure provides a composition comprising any one of, or any combination of, the TAPs defined herein (or a nucleic acid or vector encoding same); a cell expressing an MHC class I molecule and a T lymphocyte, more specifically a CD8⁺ T lymphocyte (e.g., a population of cells comprising CD8⁺ T lymphocytes).

In an embodiment, the composition further comprises a buffer, an excipient, a carrier, a diluent and/or a medium (e.g., a culture medium). In a further embodiment, the buffer, excipient, carrier, diluent and/or medium is/are pharmaceutically acceptable buffer(s), excipient(s), carrier(s), diluent(s) and/or medium (media). As used herein “pharmaceutically acceptable buffer, excipient, carrier, diluent and/or medium” includes any and all solvents, buffers, binders, lubricants, fillers, thickening agents, disintegrants, plasticizers, coatings, barrier layer formulations, lubricants, stabilizing agent, release-delaying agents, dispersion media, coatings, antibacterial and antifungal agents, isotonic agents, and the like that are physiologically compatible, do not interfere with effectiveness of the biological activity of the active ingredient(s) and that are not toxic to the subject. The use of such media and agents for pharmaceutically active substances is well known in the art (Rowe et al., Handbook of pharmaceutical excipients, 2003, 4^{th} edition, Pharmaceutical Press, London UK). Except insofar as any conventional media or agent is incompatible with the active compound (peptides, cells), use thereof in the compositions of the disclosure is contemplated. In an embodiment, the buffer, excipient, carrier and/or medium is a non-naturally occurring buffer, excipient, carrier and/or medium. In an embodiment, one or more of the TAPs defined herein, or the nucleic acids (e.g., mRNAs) encoding said one or more TAPs, are comprised within or complexed to a vesicle such as a lipid vesicle or liposome, e.g., a cationic lipid vesicle or liposome (see, e.g., Vitor M T et al., Recent Pat Drug Deliv Formul. 2013 August; 7 (2): 99-110) or suitable other carriers.

In another aspect, the present disclosure provides a composition comprising one of more of the any one of, or any combination of, the TAPs or SLP(s) defined herein (e.g., comprising or consisting of the sequence of SEQ ID NOs: 1-23 and 25-50, preferably SEQ ID NOs: 1-23 such as SEQ ID NOs: 1-17 and 22) (or nucleic acid(s) encoding said peptide(s) or SLP(s)), and a buffer, an excipient, a carrier, a diluent and/or a medium. For compositions comprising cells (e.g., APCs, T lymphocytes), the composition comprises a suitable medium that allows the maintenance of viable cells. Representative examples of such media include saline solution, Earl's Balanced Salt Solution (Life Technologies®) or PlasmaLyte® (Baxter International®). In an embodiment, the composition (e.g., pharmaceutical composition) is an “immunogenic composition”, “vaccine composition” or “vaccine”. The term “Immunogenic composition”, “vaccine composition” or “vaccine” as used herein refers to a composition or formulation comprising one or more TAPs or vaccine vector and which is capable of inducing an immune response against the one or more TAPs present therein when administered to a subject. Vaccination methods for inducing an immune response in a mammal comprise use of a vaccine or vaccine vector to be administered by any conventional route known in the vaccine field, e.g., via a mucosal (e.g., ocular, intranasal, pulmonary, oral, gastric, intestinal, rectal, vaginal, or urinary tract) surface, via a parenteral (e.g., subcutaneous, intradermal, intramuscular, intravenous, or intraperitoneal) route, or topical administration (e.g., via a transdermal delivery system such as a patch). In an embodiment, the TAP (or a combination thereof) is conjugated to a carrier protein (conjugate vaccine) to increase the immunogenicity of the TAP(s). The present disclosure thus provides a composition (conjugate) comprising a TAP (or a combination thereof), or a nucleic acid encoding the TAP or combination thereof, and a carrier protein. For example, the TAP(s) or nucleic acid(s) may be conjugated or complexed to a Toll-like receptor (TLR) ligand (see, e.g., Zom et al., Adv Immunol. 2012, 114:177-201) or polymers/dendrimers (see, e.g., Liu et al., Biomacromolecules. 2013 Aug. 12; 14 (8): 2798-806). In an embodiment, the immunogenic composition or vaccine further comprises an adjuvant. “Adjuvant” refers to a substance which, when added to an immunogenic agent such as an antigen (TAPs, nucleic acids and/or cells according to the present disclosure), nonspecifically enhances or potentiates an immune response to the agent in the host upon exposure to the mixture. Examples of adjuvants currently used in the field of vaccines include (1) mineral salts (aluminum salts such as aluminum phosphate and aluminum hydroxide, calcium phosphate gels), squalene, (2) oil-based adjuvants such as oil emulsions and surfactant based formulations, e.g., MF59 (microfluidised detergent stabilised oil-in-water emulsion), QS21 (purified saponin), AS02 [SBAS2] (oil-in-water emulsion+MPL+QS-21), (3) particulate adjuvants, e.g., virosomes (unilamellar liposomal vehicles incorporating influenza haemagglutinin), AS04 ([SBAS4] aluminum salt with MPL), ISCOMS (structured complex of saponins and lipids), polylactide co-glycolide (PLG), (4) microbial derivatives (natural and synthetic), e.g., monophosphoryl lipid A (MPL), Detox (MPL+M. Phlei cell wall skeleton), AGP [RC-529] (synthetic acylated monosaccharide), DC_Chol (lipoidal immunostimulators able to self-organize into liposomes), OM-174 (lipid A derivative), CpG motifs (synthetic oligonucleotides containing immunostimulatory CpG motifs), modified LT and CT (genetically modified bacterial toxins to provide non-toxic adjuvant effects), (5) endogenous human immunomodulators, e.g., hGM-CSF or hIL-12 (cytokines that can be administered either as protein or plasmid encoded), Immudaptin (C3d tandem array) and/or (6) inert vehicles, such as gold particles, and the like.

In an embodiment, the TAP(s) or SLP(s) (e.g., comprising or consisting of the sequence of SEQ ID NOs: 1-23 and 25-50, preferably SEQ ID NOs: 1-23) (or a nucleic acid such as a mRNA encoding said peptide(s)) or composition comprising same is/are in lyophilized form. In another embodiment, the TAP(s), SLP(s), nucleic acid(s) or composition comprising same is/are in a liquid composition. In a further embodiment, the TAP(s), SLP(s), or nucleic acid(s) is/are at a concentration of about 0.01 μg/mL to about 100 μg/mL in the composition. In further embodiments, the TAP(s), SLP(s), or nucleic acid(s) is/are at a concentration of about 0.2 μg/mL to about 50 μg/mL, about 0.5 μg/mL to about 10, 20, 30, 40 or 50 μg/mL, about 1 μg/mL to about 10 μg/mL, or about 2 μg/mL, in the composition.

As noted herein, cells such as APCs that express an MHC class I molecule loaded with or bound to any one of, or any combination of, the TAPs defined herein, may be used for stimulating/amplifying CD8⁺ T lymphocytes in vivo or ex vivo. Accordingly, in another aspect, the present disclosure provides T cell receptor (TCR) molecules capable of interacting with or binding the herein-mentioned MHC class I molecule/TAP complex, and nucleic acid molecules encoding such TCR molecules, and vectors comprising such nucleic acid molecules. A TCR according to the present disclosure is capable of specifically interacting with or binding a TAP loaded on, or presented by, an MHC class I molecule, preferably at the surface of a living cell in vitro or in vivo.

The term TCR as used herein refers to an immunoglobulin superfamily member having a variable binding domain, a constant domain, a transmembrane region, and a short cytoplasmic tail; see, e.g., Janeway et al, Immunobiology: The Immune System in Health and Disease, 3rd Ed., Current Biology Publications, p. 4:33, 1997) capable of specifically binding to an antigen peptide bound to a MHC receptor. A TCR can be found on the surface of a cell and generally is comprised of a heterodimer having α and β chains (also known as TCRα and TCRβ, respectively). Like immunoglobulins, the extracellular portion of TCR chains (e.g., α-chain, β-chain) contain two immunoglobulin regions, a variable region (e.g., TCR variable a region or Vα and TCR variable β region or Vβ; typically amino acids 1 to 116 based on Rabat numbering at the N-terminus), and one constant region (e.g., TCR constant domain a or Cα and typically amino acids 117 to 259 based on Rabat, TCR constant domain β or Cβ, typically amino acids 117 to 295 based on Rabat) adjacent to the cell membrane. Also, like immunoglobulins, the variable domains contain complementary determining regions (CDRs. 3 in each chain) separated by framework regions (FRs). In certain embodiments, a TCR is found on the surface of T cells (or T lymphocytes) and associates with the CD3 complex.

A TCR and in particular nucleic acids encoding a TCR of the disclosure may for instance be applied to genetically transform/modify T lymphocytes (e.g., CD8⁺ T lymphocytes) or other types of lymphocytes generating new T lymphocyte clones that specifically recognize an MHC class I/TAP complex. In a particular embodiment, T lymphocytes (e.g., CD8⁺ T lymphocytes) obtained from a patient are transformed to express one or more TCRs that recognize a TAP and the transformed cells are administered to the patient (autologous cell transfusion). In a particular embodiment, T lymphocytes (e.g., CD8⁺ T lymphocytes) obtained from a donor are transformed to express one or more TCRs that recognize a TAP and the transformed cells are administered to a recipient (allogenic cell transfusion). In another embodiment, the disclosure provides a T lymphocyte e.g., a CD8⁺ T lymphocyte transformed/transfected by a vector or plasmid encoding a TAP-specific TCR. In a further embodiment the disclosure provides a method of treating a patient with autologous or allogenic cells transformed with a TAP-specific TCR. In certain embodiments, TCRs are expressed in primary T cells (e.g., cytotoxic T cells) by replacing an endogenous locus, e.g., an endogenous TRAC and/or TRBC locus, using, e.g., CRISPR, TALEN, zinc finger, or other targeted disruption systems.

In another embodiment, the present disclosure provides a nucleic acid encoding the above-noted TCR. In a further embodiment, the nucleic acid is present in a vector, such as the vectors described above. In a further embodiment, the nucleic acid is an mRNA molecule.

In yet a further embodiment the use of a tumor antigen-specific TCR in the manufacture of autologous or allogenic cells for the treating of cancer (e.g., colorectal cancer) is provided.

In some embodiments, patients treated with the compositions (e.g., pharmaceutical compositions) of the disclosure are treated prior to or following treatment with an anti-tumor agent and/or immunotherapy (e.g., CAR therapy). Compositions of the disclosure include: allogenic T lymphocytes (e.g., CD8⁺ T lymphocyte) activated ex vivo against a TAP; allogenic or autologous APC vaccines loaded with a TAP; TAP vaccines and allogenic or autologous T lymphocytes (e.g., CD8⁺ T lymphocyte) or lymphocytes transformed with a tumor antigen-specific TCR. The method to provide T lymphocyte clones capable of recognizing a TAP according to the disclosure may be generated for and can be specifically targeted to tumor cells expressing the TAP in a subject (e.g., graft recipient), for example an ASCT and/or donor lymphocyte infusion (DLI) recipient. Hence the disclosure provides a CD8⁺ T lymphocyte encoding and expressing a T cell receptor capable of specifically recognizing or binding a TAP/MHC class I molecule complex. Said T lymphocyte (e.g., CD8⁺ T lymphocyte) may be a recombinant (engineered) or a naturally selected T lymphocyte. This specification thus provides at least two methods for producing CD8⁺ T lymphocytes of the disclosure, comprising the step of bringing undifferentiated lymphocytes into contact with a TAP/MHC class I molecule complex (typically expressed at the surface of cells, such as APCs) under conditions conducive of triggering T cell activation and expansion, which may be done in vitro or in vivo (i.e. in a patient administered with a APC vaccine wherein the APC is loaded with a TAP or in a patient treated with a TAP vaccine). Using a combination or pool of TAPs bound to MHC class I molecules, it is possible to generate a population CD8⁺ T lymphocytes capable of recognizing a plurality of TAPs. Alternatively, tumor antigen-specific or targeted T lymphocytes may be produced/generated in vitro or ex vivo by cloning one or more nucleic acids (genes) encoding a TCR (more specifically the alpha and beta chains) that specifically binds to a MHC class I molecule/TAP complex (i.e. engineered or recombinant CD8⁺ T lymphocytes). Nucleic acids encoding a TAP-specific TCR of the disclosure, may be obtained using methods known in the art from a T lymphocyte activated against a TAP ex vivo (e.g., with an APC loaded with a TAP); or from an individual exhibiting an immune response against peptide/MHC molecule complex. TAP-specific TCRs of the disclosure may be recombinantly expressed in a host cell and/or a host lymphocyte obtained from a graft recipient or graft donor, and optionally differentiated in vitro to provide cytotoxic T lymphocytes (CTLs). The nucleic acid(s) (transgene(s)) encoding the TCR alpha and beta chains may be introduced into a T cells (e.g., from a subject to be treated or another individual) using any suitable methods such as transfection (e.g., electroporation) or transduction (e.g., using viral vector). The engineered CD8⁺ T lymphocytes expressing a TCR specific for a TAP may be expanded in vitro using well known culturing methods.

The present disclosure provides methods for making the immune effector cells which express the TCRs as described herein. In one embodiment, the method comprises transfecting or transducing immune effector cells, e.g., immune effector cells isolated from a subject, such as a subject having a colorectal cancer (e.g., colon cancer, rectal cancer), such that the immune effector cells express one or more TCR as described herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a TCR. In this regard, the immune effector cells may be cultured before or after being genetically modified (i.e., transduced or transfected to express a TCR as described herein).

Prior to in vitro manipulation or genetic modification of the immune effector cells described herein, the source of cells may be obtained from a subject. In particular, the immune effector cells for use with the TCRs as described herein comprise T cells. T cells can be obtained from a number of sources, including peripheral blood mononuclear cells (PBMCs), bone marrow, lymph nodes tissue, cord blood, thymus issue, tissue from a site of infection, ascites, pleural effusion, spleen tissue, and tumors. In certain embodiments, T cell can be obtained from a unit of blood collected from the subject using any number of techniques known to the skilled person, such as FICOLL™ separation. In one embodiment, cells from the circulating blood of an individual are obtained by apheresis. The apheresis product typically contains lymphocytes, including T cells, monocytes, granulocyte, B cells, other nucleated white blood cells, red blood cells, and platelets. In one embodiment, the cells collected by apheresis may be washed to remove the plasma fraction and to place the cells in an appropriate buffer or media for subsequent processing. In one embodiment of the invention, the cells are washed with PBS. In an alternative embodiment, the washed solution lacks calcium and may lack magnesium or may lack many if not all divalent cations. As would be appreciated by those of ordinary skill in the art, a washing step may be accomplished by methods known to those in the art, such as by using a semi-automated flow-through centrifuge. After washing, the cells may be resuspended in a variety of biocompatible buffers or other saline solution with or without buffer. In certain embodiments, the undesirable components of the apheresis sample may be removed in the cell directly resuspended culture media. In certain embodiments, T cells are isolated from peripheral blood mononuclear cells (PBMCs) by lysing the red blood cells and depleting the monocytes, for example, by centrifugation through a PERCOLL™ gradient. A specific subpopulation of T cells, such as CD28+, CD4+, CD8+, CD45RA+, and CD45RO+ T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD8+ cells by negative selection, a monoclonal antibody cocktail typically includes antibodies to CD14, CD20, CD11b, CD16, HLA-DR, and CD4. Flow cytometry and cell sorting may also be used to isolate cell populations of interest for use in the present disclosure. PBMC may be used directly for genetic modification with the TCRs using methods as described herein. In certain embodiments, after isolation of PBMC, T lymphocytes are further isolated and in certain embodiments, both cytotoxic and helper T lymphocytes can be sorted into naive, memory, and effector T cell subpopulations either before or after genetic modification and/or expansion.

The present disclosure provides isolated immune cells such as CD8⁺ T lymphocytes that are specifically induced, activated and/or amplified (expanded) by a TAP (i.e., a TAP bound to MHC class I molecules expressed at the surface of cell), or a combination of TAPs. The present disclosure also provides a composition comprising CD8⁺ T lymphocytes capable of recognizing a TAP, or a combination thereof, according to the disclosure (i.e., one or more TAPs bound to MHC class I molecules) and said TAP(s). In another aspect, the present disclosure provides a cell population or cell culture (e.g., a CD8⁺ T lymphocyte population) enriched in CD8⁺ T lymphocytes that specifically recognize one or more MHC class I molecule/TAP complex(es) as described herein. Such enriched population may be obtained by performing an ex vivo expansion of specific T lymphocytes using cells such as APCs that express MHC class I molecules loaded with (e.g. presenting) one or more of the TAPs disclosed herein. “Enriched” as used herein means that the proportion of tumor antigen-specific CD8⁺ T lymphocytes in the population is significantly higher relative to a native population of cells, i.e., which has not been subjected to a step of ex vivo-expansion of specific T lymphocytes. In a further embodiment, the proportion of TAP-specific CD8⁺ T lymphocytes in the cell population is at least about 0.5%, for example at least about 1%, 1.5%, 2% or 3%. In some embodiments, the proportion of TAP-specific CD8⁺ T lymphocytes in the cell population is about 0.5 to about 10%, about 0.5 to about 8%, about 0.5 to about 5%, about 0.5 to about 4%, about 0.5 to about 3%, about 1% to about 5%, about 1% to about 4%, about 1% to about 3%, about 2% to about 5%, about 2% to about 4%, about 2% to about 3%, about 3% to about 5% or about 3% to about 4%. Such cell population or culture (e.g., a CD8⁺ T lymphocyte population) enriched in CD8⁺ T lymphocytes that specifically recognizes one or more MHC class I molecule/peptide (TAP) complex(es) of interest may be used in tumor antigen-based cancer immunotherapy, as detailed below. In some embodiments, the population of TAP-specific CD8⁺ T lymphocytes is further enriched, for example using affinity-based systems such as multimers of MHC class I molecule loaded (covalently or not) with the TAP(s) defined herein. Thus, the present disclosure provides a purified or isolated population of TAP-specific CD8⁺ T lymphocytes, e.g., in which the proportion of TAP-specific CD8⁺ T lymphocytes is at least about 50%, 60%, 70%, 80%, 85%, 90%, 95%, 96%, 97%, 98%, 99% or 100%.

In another aspect, the present disclosure provides an antibody or an antigen-binding fragment thereof, or a soluble TCR, that specifically binds to a complex comprising a TAP as described herein bound to an HLA molecule, such as the HLA molecules defined herein. Such antibodies are commonly referred to as TCR-like antibodies. The term “antibody or antigen-binding fragment thereof” as used herein refers to any type of antibody/antibody fragment including monoclonal antibodies (including full-length monoclonal antibodies), polyclonal antibodies, multispecific antibodies, humanized antibodies, CDR-grafted antibodies, chimeric antibodies and antibody fragments so long as they exhibit the desired antigenic specificity/binding activity. Antibody fragments comprise a portion of a full-length antibody, generally an antigen binding or variable region thereof. Examples of antibody fragments include Fab, Fab′, F(ab′)₂, and Fv fragments, diabodies, linear antibodies, single-chain antibody molecules (e.g., single-chain Fv, scFv), single domain antibodies (e.g., from camelids), shark NAR single domain antibodies, and multispecific antibodies formed from antibody fragments, single-chain diabodies (scDbs), bispecific T cell engagers (BiTEs), dual affinity retargeting molecules (DARTs), bivalent scFv-Fcs, and trivalent scFv-Fcs. Antibody fragments can also refer to binding moieties comprising CDRs or antigen binding domains including, but not limited to, V_{H} regions (V_{H}, V_{H}-V_{H}), anticalins, PepBodies, antibody-T-cell epitope fusions (Troybodies) or Peptibodies. In an embodiment, the antibody or antigen-binding fragment thereof is a single-chain antibody, preferably a single-chain Fv (scFv). In an embodiment, the antibody or antigen-binding fragment thereof comprises at least one constant domain, e.g., a constant domain of a light and/or heavy chain, or a fragment thereof. In a further embodiment, the antibody or antigen-binding fragment thereof comprises a Fragment crystallizable (Fc) fragment of the constant heavy chain of an antibody. In an embodiment, the antibody or antigen-binding fragment is a scFv comprising a Fc fragment (scFV-Fc). In an embodiment, the scFv component is connected to the Fc fragment by a linker, for example a hinge. The presence of an Fc region is useful to induce a complement-dependent cytotoxicity (CDC) or antibody-dependent cellular cytotoxicity (ADCC) response against a tumor cell.

In an embodiment, the antibody or antigen-binding fragment thereof is a multispecific antibody or an antigen-binding fragment thereof, such as a bispecific antibody or an antigen-binding fragment thereof, wherein at least one of the antigen-binding domains of the multispecific antibody or antibody fragment recognize(s) a complex comprising a TAP as described herein bound to an HLA molecule. In an embodiment, at least one of the antigen-binding domains of the multispecific antibody or antibody fragment recognize(s) an immune cell effector molecule. The term “immune cell effector molecule” refers to a molecule (e.g., protein) expressed by an immune cell and whose engagement by the multispecific antibody or antibody fragment leads to activation of the immune cells. Examples of immune cell effector molecules include the CD3 signaling complex in T cells such as CD8 T cells and the various activating receptors on NK cells (NKG2D, KIR2DS, NKp44, etc.). In a further embodiment, at least one of the antigen-binding domains of the multispecific antibody or antibody fragment recognize(s) and engage(s) the CD3 signaling complex in T cells (e.g., anti-CD3). In a further embodiment, the multispecific antibody or antibody fragment is a single-chain diabody (scDb). In a further embodiment, the scDb comprises a first antibody fragment (e.g., scFv) that binds to a complex comprising a TAP as described herein bound to an HLA molecule and a second antibody fragment (e.g., scFv) that binds to and engages an immune cell effector molecule, such as the CD3 signaling complex in T cells (e.g., anti-CD3 scFv). Such constructs may be used for example to induce the cytotoxic T cell-mediated killing of tumor cells expressing the tumor antigen/MHC complex recognized by the multispecific antibody or antibody fragment. Antibodies or antigen-binding fragments thereof may also be used as a chimeric antigen receptor (CAR) to produce CAR T cells, CAR NK cells, etc. CAR combines a ligand-binding domain (e.g., antibody or antibody fragment) that provides specificity for a desired antigen (e.g., MHC/TAP complex) with an activating intracellular domain (or signal transducing domain) portion, such as a T cell or NK cell activating domain, providing a primary activation signal. Antigen-binding fragments of antibodies, and more particularly scFv, capable of binding to molecules expressed by tumor cells are commonly used as ligand-binding domains in CAR. Thus, in another aspect, the present disclosure provides a host cell, preferably an immune cell such as a T cell or NK cell, expressing the antibody or antibody fragment (e.g., scFv) described herein.

In an embodiment, the soluble TCR is a soluble therapeutic bispecific TCR (see, e.g., Robinson et al., FEBS J. 2021 November; 288 (21): 6159-6173; Dilchert et al., Antibodies (Basel). 2022 May 10; 11 (2): 34).

The present disclosure further relates to a pharmaceutical composition or vaccine comprising the above-noted immune cell (CD8⁺ T lymphocytes, CAR T cell) or population of TAP-specific CD8⁺ T lymphocytes. Such pharmaceutical composition or vaccine may comprise one or more pharmaceutically acceptable excipients and/or adjuvants, as described above.

The present disclosure further relates to the use of any TAP or SLP (e.g., comprising or consisting of any of the sequences of SEQ ID NOs: 1-23 and 25-50, preferably SEQ ID NOs: 1-23, such as SEQ ID NOs: 1-17 and 22), nucleic acid, expression vector, T cell receptor, antibody/antibody fragment, cell (e.g., T lymphocyte, APC, CAR T cell), and/or composition according to the present disclosure, or any combination thereof, as a medicament or in the manufacture of a medicament. In an embodiment, the medicament is for the treatment of cancer, e.g., cancer vaccine. The present disclosure relates to any TAP, SLP, nucleic acid, expression vector, T cell receptor, antibody/antibody fragment, cell (e.g., T lymphocyte, APC), and/or composition (e.g., vaccine composition) according to the present disclosure, or any combination thereof, for use in the treatment of cancer e.g., as a cancer vaccine. The TAP sequences identified herein may be used for the production of synthetic peptides to be used i) for in vitro priming and expansion of tumor antigen-specific T cells to be injected into tumor patients and/or ii) as vaccines to induce or boost the anti-tumor T cell response in cancer patients, such as CRC patients. In an embodiment, the cancer (e.g., CRC) expresses one or more of the TAPs described herein.

In another aspect, the present disclosure provides the use of a TAP described herein (e.g., SEQ ID NOs: 1-23 and 25-50, preferably SEQ ID NOs: 1-23, such as SEQ ID NOs: 1-17 and 22), or a combination thereof (e.g., a peptide pool), or of one or more nucleic acid(s) encoding the TAP(s), as a vaccine for treating cancer, such as a CRC, in a subject. The present disclosure also provides the TAP described herein, or a combination thereof (e.g., a peptide pool), or of one or more nucleic acid(s) encoding the TAP(s), for use as a vaccine for treating cancer, such as a CRC, in a subject. In an embodiment, the subject is a recipient of TAP-specific T lymphocytes (e.g., CD8⁺ T lymphocytes). Accordingly, in another aspect, the present disclosure provides a method of treating cancer, such as a CRC (e.g., of reducing the number of tumor cells, killing tumor cells), said method comprising administering (infusing) to a subject in need thereof an effective amount of T lymphocytes (e.g., CD8⁺ T lymphocytes) recognizing (i.e., expressing a TCR that binds) one or more MHC class I molecule/TAP complexes (expressed at the surface of a cell such as an APC). In an embodiment, the method further comprises administering an effective amount of the TAP, or a combination thereof, or of one or more nucleic acid(s) encoding the TAP(s), and/or a cell (e.g., an APC such as a dendritic cell) expressing MHC class I molecule(s) loaded with the TAP(s), to said subject after administration/infusion of said CD8⁺ T lymphocytes. In yet a further embodiment, the method comprises administering to a subject in need thereof a therapeutically effective amount of a dendritic cell loaded with one or more TAPs. In yet a further embodiment the method comprises administering to a patient in need thereof a therapeutically effective amount of an allogenic or autologous cell that expresses a recombinant TCR that binds to a TAP presented by an MHC class I molecule.

In another aspect, the present disclosure provides the use of T lymphocytes (e.g., CD8⁺ T lymphocytes) that recognize one or more MHC class I molecules loaded with (presenting) a TAP, or a combination thereof, for treating cancer (e.g., of reducing the number of tumor cells, killing tumor cells), such as CRC, in a subject. In another aspect, the present disclosure provides the use of T lymphocytes (e.g., CD8⁺ T lymphocytes) that recognize one or more MHC class I molecules loaded with (presenting)) a TAP, or a combination thereof, for the preparation/manufacture of a medicament for treating cancer (e.g., for reducing the number of tumor cells, killing tumor cells), such as CRC, in a subject. In another aspect, the present disclosure provides T lymphocytes (e.g., CD8⁺ T lymphocytes) that recognize one or more MHC class I molecule(s) loaded with (presenting) a TAP, or a combination thereof, for use in the treatment of cancer (e.g., for reducing the number of tumor cells, killing tumor cells), such as CRC, in a subject. In a further embodiment, the use further comprises the use of an effective amount of a TAP (or a combination thereof), and/or of a cell (e.g., an APC) that expresses one or more MHC class I molecule(s) loaded with (presenting) a TAP, after the use of said TAP-specific CD8⁺ T lymphocytes.

The present disclosure also provides a method of generating an immune response against tumor cells (e.g., colorectal cancer cells) expressing human class I MHC molecules loaded with any of the TAP disclosed herein (e.g., SEQ ID NOs: 1-23 and 25-50, preferably SEQ ID NOs: 1-23, such as SEQ ID NOs: 1-17 and 22) or combination thereof in a subject, the method comprising administering cytotoxic T lymphocytes that specifically recognizes the class I MHC molecules loaded with the TAP or combination of TAPs. The present disclosure also provides the use of cytotoxic T lymphocytes that specifically recognizes class I MHC molecules loaded with any of the TAP or combination of TAPs disclosed herein for generating an immune response against tumor cells expressing the human class I MHC molecules loaded with the TAP or combination thereof.

In an embodiment, the cancer is colorectal cancer. In a further embodiment, the cancer is colon cancer. In another embodiment, the cancer is rectal cancer. In an embodiment, the colorectal cancer is characterized by microsatellite instability (MSI). In an embodiment, the colorectal cancer is characterized by microsatellite stability (MSS). In an embodiment, the colorectal cancer is characterized by RAS (e.g., KRAS) and/or RAF mutations. In another embodiment, the colorectal cancer is resistant/refractory to chemotherapy. In another embodiment, the colorectal cancer is resistant/refractory to EGFR inhibitors.

In an embodiment, the methods or uses described herein further comprise determining the HLA class I alleles expressed by the patient prior to the treatment/use, and administering or using TAPs that bind to one or more of the HLA class I alleles expressed by the patient. For example, if it is determined that the patient expresses HLA-A2*01 and HLA-B07*02, any combinations of the TAPs of SEQ ID NO:6 (that bind to HLA-A2*01) and/or SEQ ID NO:2, 21, 24, and/or 50 (that bind to HLA-B07*02) may be administered or used in the patient.

In an embodiment, the TAP, SLP, nucleic acid, expression vector, T cell receptor, antibody/antibody fragment, cell (e.g., T lymphocyte, CAR T or NK cell, APC), and/or composition according to the present disclosure, or any combination thereof, may be used in combination with one or more additional active agents or therapies to treat cancer (e.g., CRC), such as chemotherapy (e.g., vinca alkaloids, agents that disrupt microtubule formation (such as colchicines and its derivatives), anti-angiogenic agents, therapeutic antibodies, EGFR targeting agents, tyrosine kinase targeting agent (such as tyrosine kinase inhibitors), transitional metal complexes, proteasome inhibitors, antimetabolites (such as nucleoside analogs), alkylating agents, platinum-based agents, anthracycline antibiotics, topoisomerase inhibitors, macrolides, retinoids (such as all-trans retinoic acids or a derivatives thereof), geldanamycin or a derivative thereof (such as 17-AAG), surgery, immune checkpoint inhibitors or immunotherapeutic agents (e.g., PD-1/PD-L1 inhibitors such as anti-PD-1/PD-L1 antibodies, CTLA-4 inhibitors such as anti-CTLA-4 antibodies, B7-1/B7-2 inhibitors such as anti-B7-1/B7-2 antibodies, TIM3 inhibitors such as anti-TIM3 antibodies, BTLA inhibitors such as anti-BTLA antibodies, CD47 inhibitors such as anti-CD47 antibodies, GITR inhibitors such as anti-GITR antibodies), antibodies against tumor antigens (e.g., anti-CD19, anti-CD22 antibodies), cell-based therapies (e.g., CAR T cells, CAR NK cells), and cytokines such as IL-2, IL-7, IL-21, and IL-15. In an embodiment, the TAP, nucleic acid, expression vector, T cell receptor, cell (e.g., T lymphocyte, APC), and/or composition according to the present disclosure is administered/used in combination with an immune checkpoint inhibitor. In an embodiment, the TAP, nucleic acid, expression vector, T cell receptor, cell (e.g., T lymphocyte, APC), and/or composition according to the present disclosure is administered/used in combination one or more therapies used for the treatment of CRC (e.g., surgery, chemotherapy (e.g., using 5-fluorouracil, capecitabine, oxaliplatin, irinotecan, raltitrexed, trifluridine, tipiracil), radiation therapy, bevacizumab, cetuximab, panitumumab, regorafenib.

The additional therapy may be administered prior to, concurrent with, or after the administration of the TAP, SLP, nucleic acid, expression vector, T cell receptor, antibody/antibody fragment, cell (e.g., T lymphocyte, CAR T or NK cell, APC), and/or composition according to the present disclosure.

### EXAMPLES

The present disclosure is illustrated in further details by the following non-limiting examples.

### Example 1: Experimental Procedures

Cell lines. Four colorectal cancer cell lines [COLO 205 (ATCC® CCL-222™), HCT 116 (ATCC® CCL-247™), RKO (ATCC® CRL-2577™), SW620 [SW-620] (ATCC® CCL-227™)] and 1 normal fetal small intestine cell line [HIEC6 (ATCC® CRL3266™)] were obtained from the American Type Culture Collection (ATCC). COLO205, HCT116, and SW620 were grown in RPMI-1640 (Gibco) supplemented with 10% Fetal bovine serum (FBS), RKO was grown in Eagle's Minimum Essential Medium (EMEM) (ATCC®) supplemented with 10% FBS, and HIEC-6 was grown in OptiMEM® 1 Reduced Serum Medium (Gibco) supplemented with 20 mM HEPES (Gibco), 10 mM GlutaMAX® (Gibco), 10 ng/mL epidermal growth factor (EGF) (Gibco), and FBS to a final concentration of 4%. All cells were maintained at 37° C. with 5% CO₂. For collection, cells were rinsed with warm PBS before being trypsinized with TrypLET Express Enzyme (1×) (Gibco) for 5-15 minutes at 37° C. with 5% CO₂. Harvested material was then spun at 1000 rpm for 5 minutes, rinsed once with warm PBS, then resuspended in ice-cold PBS. After cell count, replicates of 2×10⁸ CRC cells were pelleted and frozen at −80° C. until further use. MHC class I surface density of the CRC cell lines was determined by Qifikit™ (Agilent) using the W6/32 anti-HLA class I antibody (BioXCell), according to the manufacturer's instructions.

Primary tissues. Six pairs of primary human samples consisting of matched colon adenocarcinoma tumor and normal adjacent tissue (NAT) were purchased from Tissue Solutions. Tissue samples were taken from patients receiving surgery as first line of treatment and were flash-frozen in liquid nitrogen. More information about primary tissue samples can be found in Table 2.

RNA extraction. For RNA extraction of cell lines, 1-2 million cells were collected and washed once with ice-cold PBS. The cells were then resuspended in Trizol™ (Invitrogen). For cell lines and primary tissue samples, total RNA was isolated using the AllPrep™ DNA/RNA/miRNA Universal kit (Qiagen) or the RNeasy™ Mini kit (Qiagen) as recommended by the manufacturer.

RNA sequencing. 500 ng of total RNA was used for library preparation. RNA quality control was assessed with the Bioanalyzer™ RNA 6000 Nano assay on the 2100 Bioanalyzer™ system (Agilent Technologies) and all samples had a RIN above 8. Libraries were prepared with the KAPA mRNAseq Hyperprep™ kit (Roche). Ligation was made with Illumina dual-index UMI (IDT). After being validated on BioAnalyzer™ DNA1000 chip and quantified by QuBit and qPCR, libraries were pooled to equimolar concentration and sequenced with the Illumina Nextseq500 using the Nextseq™ High Output 150 (2×75 bp) cycles kit. A mean of 129 and 95 million paired-end PF reads were generated for the cell lines and tissue samples, respectively. Library preparation and sequencing was performed at the Genomic Platform of the Institute for Research in Immunology and Cancer (IRIC).

Bioinformatic analyses. Sequences were trimmed using Trimmomatic version 0.35 (17) and aligned to the reference human genome version GRCh38 (gene annotation from Gencode version 33, based on Ensembl 99) using STAR version 2.7.1a (18). Gene expressions were obtained both as readcount directly from STAR as well as computed using RSEM (19) in order to obtain normalized gene and transcript level expression, in TPM values, for these stranded RNA libraries.

HLA genotyping. HLA genotyping of cell lines and tissues was performed using OptiType (https://github.com/FRED-2/OptiType) (20).

Microsatellite instability prediction. MSI status of the primary tumor samples was predicted using the MSIsensor program using paired tumor and NAT (https://github.com/ding-lab/msisensor) (21).

Differential expression analysis. DESeq2 version 1.22.2 (22) was used to normalize gene readcounts and compute differential expression between tumor and normal samples. Principal component analyses (PCA) were generated using normalized log readcounts for the first two most significant components. For differential expression analysis of the cell lines, fold changes were computed between the mean expression of the four CRC cell lines compared to the normal cell line (HIEC-6). Significant differentially expressed genes (DEGs) are (those with padj lower than 0.05) and with |log 2 fold change|>1 were considered for GO terms using the Metascape tool (23). For paired differential expression analysis of the tissues, TPM normalized values were used to compare tumor/NAT pairs. As only a single replicate of the tissues was sequenced, rather than filtering by adjusted p-value, only genes that were significantly differentially expressed in all six subjects for GO term analysis with |log 2 fold change|>1 were selected. When examining differentially expressed genes between MSS and MSI tissues, the same fold change thresholds were applied. For GO term analysis of MSI DEGs, genes were selected that were exclusively differentially expressed in both MSI tissues (i.e., not considered DEGs in any MSS tissues). For GO term analysis of MSS DEGs, genes were considered if they were differentially expressed in 3 or more MSS tissues.

Transcriptome analysis of tissue samples. The proportion of various biotypes in the transcriptome of tissue samples was determined as previously described (24). Briefly, following quantification and alignment of Ensembl annotated transcripts by Kallisto (19), transcripts and repetitive elements were annotated using a Kallisto index containing Ensembl annotated transcripts supplemented with genetic repeat identifications from the USCS Table Browser GRCh38 repeat masker database (25).

Mutation profiles/‘genetic variant annotation’. Genetic variant calling was performed for both cell line and primary biopsies using SNPEff (https://pcingola.github.io/SnpEff/#snpeff) (26).

Database generation. Global cancer databases were constructed as previously described (16). In brief, RNA-seq reads were trimmed using Trimmomatic version 0.35 (17) and aligned to the reference human genome version GRCh38 (gene annotation from Gencode version 33, based on Ensembl 99) using version STAR 2.7.1a (18). Kallisto (https://pachterlab.github.io/kallisto) was used to quantify transcript expression in TPM (19). Sample-specific exomes were constructed by integrating single nucleotide variants (quality >20) identified with Freebayes (https://github.com/ekg/freebayes) into PyGeno (27). Annotated open reading frames with TPM>0 were then translated in silico and added to the canonical proteome in fasta format. To generate the cancer-specific proteome, RNA-seq reads were cut into 33-nucleotide sequences known as kmers and only kmers present <2 in mTECs or matched NAT for cell lines and tissues, respectively, were kept. Overlapping kmers were assembled into contigs, which were then 3-frame translated in silico. Of note, short peptide sequences generated through the kmer approach were then concatenated into longer sequences of approximately ten thousand amino acids. These peptides were concatenated using the ‘JJ’ sequence as a separator, which is recognized internally by the PeaksX+ software to split sequences upon occurrence of this sequence. Then, the canonical proteome and the cancer-specific proteome were concatenated to create the global cancer databases. Cell line databases consisted of 3.38×10⁶ sequences on average.

Isolation of MAPS. CRC cell line pellet samples (2×10₈ cells per replicate, 4 replicates per cell line) were resuspended with PBS up to 2 mL and then solubilized by adding 2 mL of ice-cold 2× lysis buffer (1% w/v CHAPS). Tumor and normal adjacent tissue samples (between 455 mg and 693 mg) were cut into small pieces (cubes, ˜3 mm in size) and 5 ml of ice-cold PBS containing protein inhibitor cocktail (Sigma, cat #P8340-5 ml) was added. Tissues were first homogenized twice for 20 seconds using an Ultra Turrax T25 homogenizer (IKA-Labortechnik) set at speed 20000 rpm and then 20 seconds using an Ultra Turrax T8 homogenizer (IKA-Labortechnik) set at speed 25000 rpm. Then, 550 μl of ice-cold 10× lysis buffer (5% w/v CHAPS) was added to each sample. After 60-minute incubation with tumbling at 4° C., tissue samples and CRC cell line samples were spun at 10000 g for 30 minutes at 4° C. Supernatants were transferred into new tubes containing 1 mg of W6/32 antibody covalently-cross-linked protein A magnetic beads and MAPs were immunoprecipitated as previously described (28). MAP extracts were then dried using a Speed-Vac and kept frozen before MS analyses.

TMT labeling. MAP extracts were resuspended in 200 mM Hepes buffer pH 8.1. 50 μg of TMT reagent (Thermo Fisher Scientific) in anhydrous acetonitrile was added to samples as follows: CRC cell line replicates were labeled with TMT6plex (lot #UG287166) channels TMT6-126-129; Tissue samples were labeled with TMT10plex (lot #UH285228) −126 (NAT) and −127N (tumor). Samples were gently vortexed and reacted at room temperature for 1.5 hours. Samples were then quenched with 50% hydroxylamine for 30 minutes at room temperature, then were diluted with 4% FA/H2O. CRC cell line replicates and individual NAT-tumor pairs were combined. Samples were then desalted on homemade C18 membrane (Empore) columns and stored at −20° C. until injection. To quantify MAPs of interest in primary tissue samples, synthetic peptides at concentrations of 0.75 to 192 fmoles were labeled with TMT 10plex channels −128N, −128C, −129N, −129C, −130N, −130C, −131, while the NAT and CRC tissues were labeled with −126 and 127N, respectively. Note that the channel −127C was left empty to assess contamination between channels.

Liquid Chromatography-tandem MS analyses. Dried peptide extracts were resuspended in 4% FA and loaded on a homemade C18 analytical column (20 cm×150 μm i.d. packed with C18 Jupiter Phenomenex) with a 106-minute gradient from 0% to 30% ACN (0.2% FA) and a 600 nL/min flow rate on an EASY-nLC II system. Samples were analyzed with an Orbitrap Exploris 480 spectrometer (Thermo Fisher Scientific) in positive ion mode with Nanoflex source at 2.8 kV. Each full MS spectrum, acquired with a 240,000 resolution was followed by 20 MS/MS spectra, where the most abundant multiply charged ions were selected for MS/MS sequencing with a resolution of 30,000, an automatic gain control target of 100%, an injection time of 700 ms, and collisional energy of 40%.

MAP Identification. Database searches were conducted using the PeaksX+ software (Bioinformatics Solutions Inc.) (29). Error tolerances for precursor mass and fragment ions were set to 10.0 ppm and 0.01 Da, respectively. A non-specific digest mode was used. TMT6plex or 10plex was set as a fixed PTM, and variable modifications included phosphorylation (STY), Oxidation (M), Deamidation (NQ), and TMT6plex or 10plex STY. Peaks searches were then loaded into MAPDP (30), which was used to apply the following filters: selecting peptides of 8-11 amino acids in length, with rank eluted ligand threshold≤2% based on NetMHCpan-4.1b predictions, using a 5% FDR.

Quantification of MAP coding sequences in RNA-Seq data. MAP coding sequences (MCSs) were quantified in RNA-seq data as previously described (31). Briefly, MCSs were reverse translated into all possible nucleotide sequences with an in-house python script (deposited on Zenodo at DOI: 3739257). The nucleotide sequences were then mapped onto the genome with GSNAP (32) to determine all possible genomic locations able to code for a given MAP. MCSs were also mapped onto the transcriptome to account for MAPs overlapping splice sites, and portions of the transcriptome corresponding to these MAPs were then also mapped onto the reference genome with GSNAP. For MAPs of interest, genomic alignment of all reads containing the MCS was performed. GSNAP output was filtered to keep only perfect matches between the sequence and reference, resulting in a file containing all possible genomic regions able to code for a given MAP. The number of reads containing the MCSs at their respective genomic locations in each desired RNA-Seq sample (such as CRC and NAT, GTEx, or TCGA samples), aligned on the reference genome with STAR, was summed. Lastly, all read counts for a given MAP were summed and normalized on the total number of reads sequenced in each sample of interest to obtain a reads-per-hundred-million (RPHM) count.

Determination of MAP source transcripts. To investigate what proportion of tissue sample MAPs were derived from certain transcript biotypes, the most abundant putative source transcript based on kmer-per-hundred-million (KPHM) quantification was determined. For peptides from the cancer-specific (kmer) database, the MCSs were reverse translated into all possible nucleotide sequences and all possible genomic regions able to code for a given MAP were identified (see ‘Quantification of MAP coding sequences in RNA-Seq data’ above). Finally, Kallisto was used to determine the most expressed transcript at that location, which was then assigned as the most probable transcript for the given peptide. Peptides that had more than one putative source transcript were excluded from the analysis.

Identification of TSA candidates. TSA candidates were identified through a stringent TSA identification pipeline. First, MAPs underwent peptide classification in which the peptide sequence accessions were retrieved from the protein database and used to extract the nucleotide sequences of each peptide. RNA-Seq data from each cancer and normal samples were transformed into 24-nucleotide-long k-mer databases with Jellyfish 2.2.3 (using the -C option) and used to query each TSA candidate coding sequence's 24-nucleotide-long k-mer set. The number of reads fully overlapping a given peptide-coding sequence was estimated using the k-mer set's minimum occurrence (kmin), as in general, one k-mer always originates from a single RNA-Seq read. This kmin value was then transformed into several k-mers detected per 10⁸ reads sequenced (kphm) using the following formula: kphm=(kmin×10₈) rtot, with rtot representing the total number of reads sequenced in a given RNA-Seq experiment. Peptides were kept only if their RNA coding sequences were expressed at least 10-fold higher in cancer than in normal (pooled mTEC samples for cell lines, matched NAT for tissues), and expressed <2 KPHM in normal. Subsequent filtering removed any peptides with indistinguishable isoleucine/leucine variants; a peptide with an IL variant was kept only if the most expressed variant met the above-mentioned criteria. The MCSs of the remaining peptides were quantified in RNA-seq data as described above and were kept only if their expression was <8.55 RPHM in mTECs and other normal tissues (GTEx). Genomic localization for each peptide was assigned by mapping reads containing each MCS to the reference genome (GRCh38.99) using BLAT (https://genome.ucsc.edu/cgi-bin/hgBlat, Kent W J. BLAT—the BLAST-like alignment tool. Genome Res. 2002 April; 12 (4): 656-64. PMID: 11932250). Peptides were excluded if the genomic localization was unclear or if they mapped to a hypervariable region (HLA, Ig, or T cell receptor (TCR) genes). Finally, the MS/MS spectra of the remaining candidates were manually validated. Peptides were classified as mTSAs if their amino acid sequence was different from the reference, and if the mutation was not a known germline polymorphism. Peptides were classified as aeTSAs if they were overexpressed ≥10-fold in tumor compared to normal and ≤0.2 KPHM in mTECs (and NAT in the case of tissues) and as TAAs if they were overexpressed ≥10-fold in cancer but the expression in mTECs and/or NAT was >0.2 KPHM. Ultimately, the transcript of origin of each TSA/TAA was attributed by selecting the most highly expressed peptide-overlapping transcript from the kallisto quantification file (see Database Generation section).

Intertumoral sharing. To examine the intertumoral distribution of TSA and TAA sequences in other CRC tumors, the log (RPHM+1) expression of the peptide coding sequences in 151 colon adenocarcinoma (COAD) samples from TCGA was determined (see ‘Quantification of MAP coding sequences in RNA-Seq data’).

Immunogenicity prediction. The predicted immunogenicity of MAPs of interest was determined with the R package Repitope v3.0.1 (https://github.com/masato-ogishi/Repitope) (34).

Validation of TSA peptide candidates. Synthetic peptides of TSA and select TAA sequences were obtained from Genscript. Synthetic peptides were solubilized in DMSO to a concentration of 1 nmol/μL and all synthetic peptides were combined in a stock solution at a concentration of 10 pmol/μL. The stock solution was desalted in aliquots of 150 pmol on homemade C18 membrane (Empore) columns and dried using a Speed-Vac. Dried peptide extracts were labeled with a TMT10plex channel as described (see ‘TMT labeling’ section), desalted, and dried down in Speed-Vac. Labeled synthetic peptides were resuspended in 4% FA and 1 pmol of each synthetic peptide was loaded on a homemade C18 analytical column (20 cm×150 μm i.d. packed with C18 Jupiter Phenomenex) with a 76-minute gradient from 0% to 30% ACN (0.2% FA) and a 600 nL/min flow rate on an EASY-nLC II system. Samples were analyzed with an Orbitrap Exploris 480 spectrometer (Thermo Fisher Scientific) in positive ion mode with Nanoflex source at 2.8 kV. Each full MS spectrum was acquired with a 120 000 resolution, and an inclusion list was used to select ions for fragmentation with 40% collision energy and an isolation window of 1 m/z. MS/MS were acquired with a resolution of 30 000. MS/MS correlations were computed as previously described (17). Briefly, expected peptide fragments were computed with pyteomics v4.0.1 (https://bitbucket.org/levitsky/pyteomics) and reproducibly detected peptide fragments were identified. Root scaled intensities of these fragments were correlated between endogenous and synthetic peptide scan pairs and SciPy v1.2.1 (https://www.scipy.org/) was used to compute Pearson correlation coefficient, p-value, and confidence intervals. Mirror plots of the scan pair with the lowest p-value were generated for each peptide using spectrum_utils v0.2.1 (https://github.com/bittremieux/spectrum_utils).

Relative quantification. To relatively quantify MAPs of interest in primary tissue samples, synthetic peptides at concentrations of 10, 100, or 1000 fmol labeled with TMT 10plex-129N, 130N, and 131N, respectively, were spiked into remaining purified MAPs from NAT and CRC tissue samples labeled with TMT10plex-126 and 127N, respectively. Note that the channel TMT 10plex-127C was left empty to assess contamination. Samples were analyzed with an Orbitrap Fusion Tribrid spectrometer (Thermo Fisher Scientific) in positive ion mode with Nanoflex source at 2.4 kV. For synchronous precursor selection MS3 (SPS-MS3), full MS scans were acquired with a range of 300-1000 m/z, Orbitrap resolution of 120 000, automatic gain control (AGC) of 5.0e5, and a maximum injection time of 50 ms, using an inclusion list for the peptides of interest. A 3s top speed approach for MS2 was used in the ion trap, with an isolation window of 0.4m/z, collision induced dissociation of 35%, a ‘normal’ ion trap scan rate mode, 2.0e4 AGC target, and 50 ms maximum injection time. This was followed by the selection of eight synchronous precursor ions for MS3 acquisition, which was done with a scan range of 110-500 m/z, Orbitrap resolution of 50 000, AGC of 1.0e5, maximum injection time of 300 ms, an isolation window of 2.0m/z, and 65% HCD collision energy. LC-MS instrument was controlled using Xcalibur version 4.4 (Thermo Fisher Scientific, Inc). Error tolerances for precursor mass and fragment ions were set to 10.0 ppm and 0.5 Da, respectively. A non-specific digest mode was used. TMT10plex was set as a fixed PTM, and variable modifications included phosphorylation (STY), Oxidation (M), Deamidation (NQ), and TMT10plex STY. For quantification, PSMs were filtered to exclude those with contamination in the TMT10plex-127C channel, and to select those within the 70th intensity percentile. MS2 precursor profiles and intensity profiles of all relevant channels were manually inspected to select peptides for quantification. Intensity ratios for each peptide were calculated using the average TMT10plex-127N and TMT10plex-126 intensities of good quality PSMs.

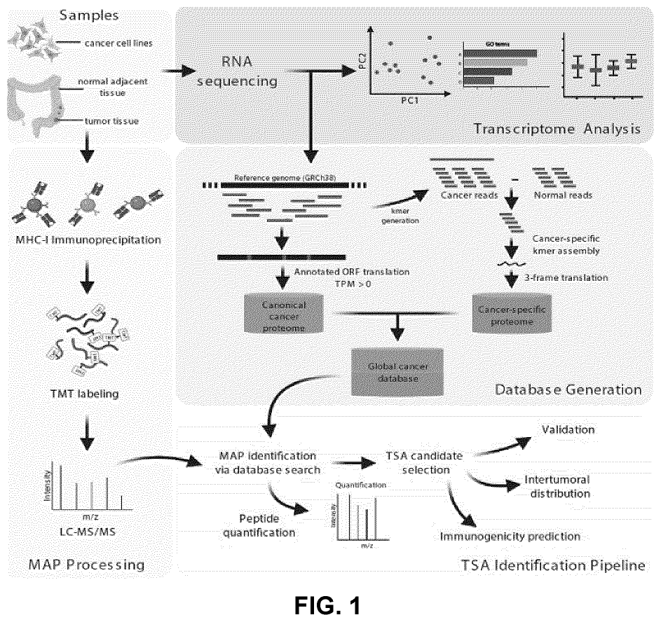

Data analysis and visualization. FIG. 1 was generated with BioRender.com. Majority of other figures were created with Python v3.7.6, R v3.6.3, or Origin (Pro) 2019b. R packages include: Repitope v3.0.1 (https://github.com/masato-ogishi/Repitope) (34), UpsetR v1.4.0 (https://github.com/hms-dbmi/UpSetR) (35), GSVA v1.38.2 (https://github.com/rcastelo/GSVA) (36), ESTIMATE v1.0.13 (https://bioinformatics.mdanderson.org/estimate/) (37).

Experimental Design and Statistical Rationale. To effectively elucidate the MHC I immunopeptidome of colorectal cancer, 4 CRC cell lines were selected and 6 samples from human subjects consisting of both matched tumor and normal adjacent tissue (NAT) were acquired. NAT was used as an approximation of healthy tissue, as it is the most effective control for each respective tumor. Since no matched samples were available for cell lines, a pool of 6 mTEC samples was used in the creation of global cancer databases, to obtain a wide berth of approximate normal RNA expression. All instances of p-values are determined using two-sample t-test, except in the determination of significance for immunogenicity scores, in which case the Mann-Whitney test was used as the data did not have a normal distribution, as determined by the Shapiro test. For t-tests, f-tests were performed to determine whether the dataset had significant variation; if yes, then the t-test assuming variation was used, and otherwise the t-test assuming no variation was used. For CRC-derived cell lines, four technical replicates of 2×10⁸ cells were prepared, which were TMT labeled and multiplexed prior to injection. Due to limited tissue material, half of the purified MAPs from primary samples were injected to obtain global immunopeptidomic data, and the remaining sample was used for targeted analysis with synthetic peptides to confirm the sequences and abundance of putative TSAs and select TAAs. To select high quality PSMs for quantification, those of low intensity or with contamination in an empty TMT channel were excluded. Further, only peptides with favorable MS2 precursor and intensity profiles were quantified.

### Example 2: Immunopeptidomic Analyses Using a Proteogenomic Approach

To determine the composition of the immunopeptidome of colorectal cancer, a collection of samples comprised of 4 colorectal cancer-derived cell lines and 6 sets of primary adenocarcinoma samples, which consist of matched tumour and normal adjacent tissue (Tables 1 and 2), were analyzed. Paired-end RNA sequencing (RNA-seq) allowed the creation of a global cancer database, consisting of a canonical cancer proteome as well as a cancer-specific proteome for each sample, created through the generation of cancer-specific kmers which are translated into 3 reading frames to encompass non-canonical sequences from any genomic origin (FIG. 1). Medullary thymic epithelial cells (mTECs) present peripheral antigens in the thymus and mediate the negative selection of auto-reactive T-cells (38). In the case of CRC-derived cell lines, cancer-specific kmers were obtained following the subtraction of mTEC-derived sequences, which approximate the expression of these sequences in healthy tissues. For the primary tissue samples, the cancer-specific kmers were generated following subtraction of the sequences from matched NAT. This approach enabled the determination of sequences that are expressed in tumor and not observed in healthy colon tissue of the same individual. In addition to database construction, RNA-seq data were also used for transcriptomic analysis, including GO term analysis, investigation of immune infiltration, mutation profiling, and determination of transcript abundance (FIG. 1).

TABLE 1
Description of CRC-derived cell lines.

| Cell | | | | | MHC I | HLA | Mutations of |
|---|---|---|---|---|---|---|---|
| line | Tissue | Morphology | Disease | Biomarkers | molecule/cell | genotyping | interest |
| Colo205 | Colon; | Epithelial | Dukes' type D, | MSS, | 1.44 × 10⁵ ± | HLA-A*01:01 | BRAF |
| | derived from | | colorectal | CIMP | 0.00282 × 10⁵ | HLA-A*02:01 | (V600E), |
| | metastatic | | adenocarcinoma | | | HLA-B*07:02 | SMAD4, |
| | site: ascites | | | | | HLA-B*08:01 | TP53 |
| | | | | | | HLA-C*07:01 | |
| | | | | | | HLA-C*07:02 | |
| HCT116 | Colon | Epithelial | Colorectal | MSI, CIMP | 5.07 × 10⁵ ± | HLA-A*01:01 | RAS |
| | | | carcinoma | | 0.30 × 10⁵ | HLA-A*02:01 | (G13D), |
| | | | | | | HLA-B*18:01 | PI3CA, |
| | | | | | | HLA-B*45:01 | CDKN2A, |
| | | | | | | HLA-C*05:01 | CTNNB1 (B- |
| | | | | | | HLA-C*07:01 | catenin) |
| RKO | Colon | Epithelial | Carcinoma | MSI, CIMP | 2.82 × 10⁵ ± | HLA-A*03:01 | BRAF |
| | | | | | 0.11 × 10⁵ | HLA-B*18:01 | (V600E), |
| | | | | | | HLA-C*07:01 | PI3CA |
| SW620 | Colon; | Epithelial | Dukes' type C, | MSS, CIN | 1.69 × 10⁵ ± | HLA-A*02:01 | APC, RAS |
| | derived from | | colorectal | | 0.0017 × 10⁵ | HLA-A*24:02 | (G12V), |
| | metastatic | | adenocarcinoma | | | HLA-B*07:02 | SMAD4, |
| | site: lymph | | | | | HLA-B*15:18 | TP53 |
| | node | | | | | HLA-C*07:02 | |
| | | | | | | HLA-C*07:04 | |

TABLE 2
Description of primary tumor and matched NAT

| Sample ID | Sex | Age | Ethnic background | Matrix | Diagnosis |
|---|---|---|---|---|---|
| S1_N | | | | colon | NAT |
| S1_T | F | 73 | Caucasian | cecum | cancer |
| S2_N | | | | colon | NAT |
| S2_T | M | 60 | Caucasian | Sigmoid | Cancer |
| S3_N | | | | colon | NAT |
| S3_T | F | 63 | Caucasian | sigmoid | cancer |
| S4_N | | | | colon | NAT |
| S4_T | F | 85 | Caucasian | sigmoid | cancer |
| S5_N | | | | colon | NAT |
| S5_T | F | 43 | Caucasian | ascending | cancer |
| | | | | colon | |
| S6_N | | | | colon | NAT |
| S6_T | F | 48 | Caucasian | sigmoid | cancer |

| Sample | Histological | | Tumor | Mutations | |
|---|---|---|---|---|---|
| ID | diagnosis | Stage | content % | of interest | HLA |
| S1_N | | | | | HLA-A*24:02 |
| | | | | | HLA-B*07:02 HLA-B*35:01 |
| | | | | | HLA-C*04:01 HLA-C*07:02 |
| S1_T | adenocarcinoma | IIC | 100 | KRAS G12D | |
| S2_N | | | | | HLA-A*02:01 HLA-A*03:02 |
| | | | | | HLA-B*27:05 HLA-B*58:01 |
| | | | | | HLA-C*02:02 HLA-C*07:01 |
| S2_T | Adenocarcinoma | IIA | 95 | | |
| S3_N | | | | | HLA-A*01:01 HLA-A*32:01 |
| | | | | | HLA-B*38:01 HLA-B*50:01 |
| | | | | | HLA-C*06:02 HLA-C*12:03 |
| S3_T | adenocarcinoma | IIA | 100 | KRAS Q61H | |
| S4_N | | | | | HLA-A*01:01 HLA-A*11:01 |
| | | | | | HLA-B*15:01 HLA-B*57:01 |
| | | | | | HLA-C*03:03 HLA-C*06:02 |
| S4_T | adenocarcinoma | IIA | 100 | KRAS G12D | |
| S5_N | | | | | HLA-A*03:01 HLA-A*30:01 |
| | | | | | HLA-B*13:02 HLA-B*52:01 |
| | | | | | HLA-C*06:02 HLA-C*12:02 |
| S5_T | adenocarcinoma | IIA | 95 | | |
| S6_N | | | | | HLA-A*03:01 HLA-A*23:01 |
| | | | | | HLA-B*07:02 HLA-B*18:01 |
| | | | | | HLA-C*07:01 HLA-C*07:02 |
| S6_T | adenocarcinoma | IIA | 95 | | |

MHC I: peptide complexes were isolated through immunoprecipitation, and the eluted MHC I-associated peptides (MAPs) were labeled with tandem mass tag (TMT) isobaric labeling reagent, as TMT labeling was recently shown to enhance the detection of MAPs by increasing their charge state and hydrophobicity (37). MAPs were then sequenced and analyzed by liquid chromatography tandem mass spectrometry (LC-MS/MS) using the personalized cancer databases generated through RNA-seq. Identified MAPs then underwent a rigorous series of classifications and validations to identify putative TSAs and TAAs. Tumor antigens identified in CRC tissues were then validated and quantified with synthetic peptides to determine to what extent they were overexpressed at the cell surface of tumors, and their predicted immunogenicity and intertumoral distribution were also investigated to obtain a sense of their clinical potential (FIG. 1).

In the present study, 4 CRC-derived cell lines with different alleles and characteristics were used as summarized in Table 1. HCT116 and RKO are derived from primary tumors and are characterized by microsatellite instability (MSI), whereas Colo205 and SW620 are derived from metastases of ascites and lymph node, respectively, and are both microsatellite stable (MSS). These cell lines have a wide range of MHC I surface expression ranging from 1.44×10⁵ to 5.07×10⁵ MHC I molecules/cell and a diversity of HLA alleles. Among the 4 cell lines are mutations in several key genes, such as BRAF, RAS, SMAD4, TP53, and PI3CA. These cell lines have a varying MHC I surface expression ranging from 1.44×10⁵ to 5.07×10⁵ MHC I molecules/cell, as determined by Qifikit, and a diversity of HLA alleles which were identified using OptiType, an HLA genotyping tool that uses RNA-Seq data to predict a sample's HLA alleles, in combination with the HLA alleles for these cell lines documented in the literature (Table 1) (22).

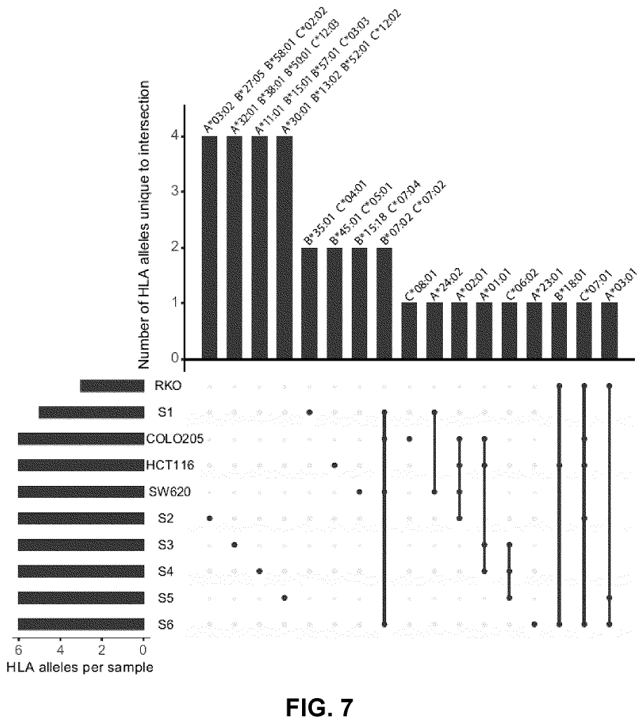

All of the primary tumor samples are derived from stage II adenocarcinomas, which vary only slightly in tumor grade and TNM (tumor-node-metastases) classification (Table 2). The primary samples had a tumor content of 95-100% and an average mass of 0.6625 grams. The tumors are all derived from the sigmoid colon, with the exception of S1 (cecum) and S5 (ascending colon). All patients are female, with the exception of S2, and the patients range in age from 43-85, with a mean age of 62. Similar to the cell lines, the tissue samples also possess a variety of HLA alleles. A visualization of the number of HLA alleles unique to or shared by cell line and tissue samples is available in FIG. 7. There is an average of 1.3 and 3.2 unique alleles per cell line and tissue, respectively.

### Example 3: Transcriptomic Characterization of the CRC Samples

Because the outcome for CRC patients within a given disease stage differs greatly based on the molecular characteristics of the tumor (40, 41), RNA sequencing data were used to characterize the molecular heterogenicity of the samples. After first examining the mutational status of key biomarkers (such as KRAS, NRAS, or BRAF) which are commonly used to guide therapeutic decisions and prognosis in the clinics (40, 41) (Table 1), the microsatellite statuses of cell lines and primary samples were respectively determined from the literature (42, 43) and using the MSIsensor package (46) to expand the knowledge of the molecular characteristics of these samples. While MSI is found in a limited subset of CRC tumors (i.e., 15% of sporadic CRC and 90% of nonpolyposis colorectal cancer) (47), in this study, 50% of the tumorigenic cell lines and 33% of the primary biopsies present this phenotype (Table 3). Although several elements in the literature suggest that MSI and MSS tumors are immunologically different (5, 11, 48, 49), this study provides a comparison of MSI and MSS colorectal tumors at the immunopeptidomic level.

TABLE 3
MSISensor results for CRC primary tissues.

| | | | Number of | | | |
|---|---|---|---|---|---|---|
| | | Number of sites | sites with | Number of | Number of MSI | |
| | Number of | with enough | enough | MSI sites | sites (somatic) | Class(MSI > |
| Sample | total sites | coverage | coverage (%) | (somatic) | (%) | 3.5%) |
| S1 | 1011195 | 111243 | 11 | 226 | 0.20 | MSS |
| S2 | 1011195 | 69004 | 7 | 197 | 0.29 | MSS |
| S3 | 1011195 | 56779 | 6 | 104 | 0.18 | MSS |
| S4 | 1011195 | 45848 | 5 | 177 | 0.39 | MSS |
| S5 | 1011195 | 78340 | 8 | 8267 | 10.55 | MSI |
| S6 | 1011195 | 60715 | 6 | 3085 | 4.08 | MSI |

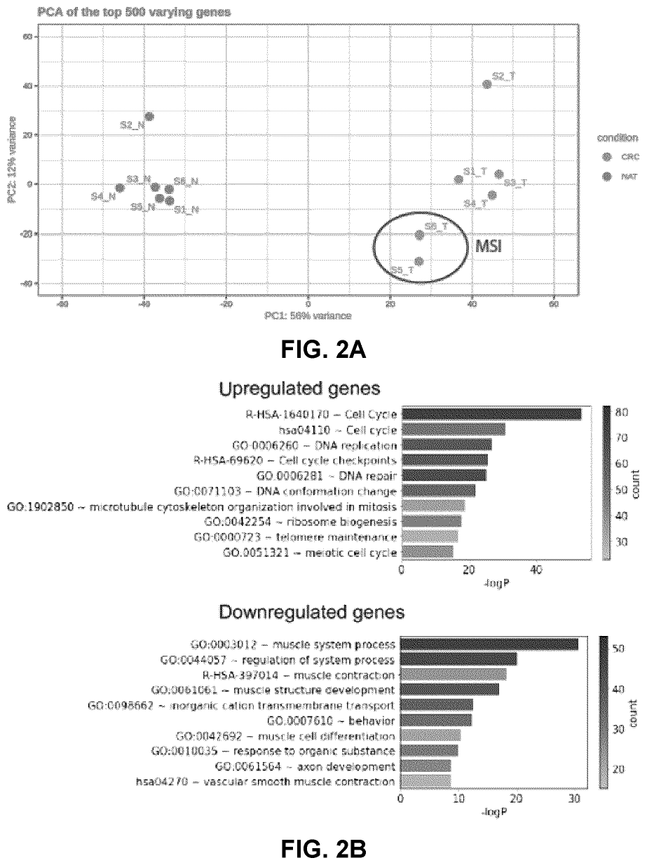

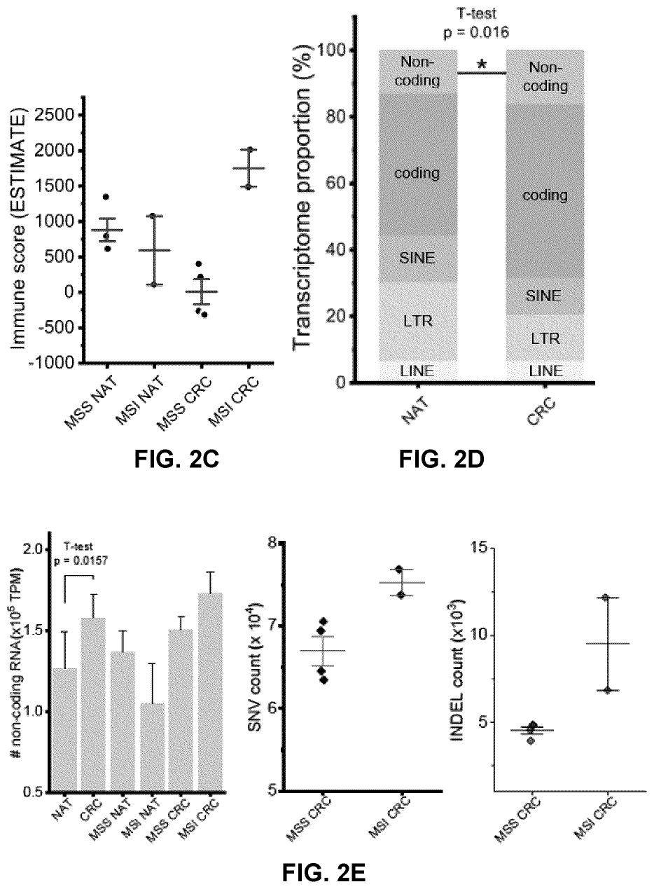

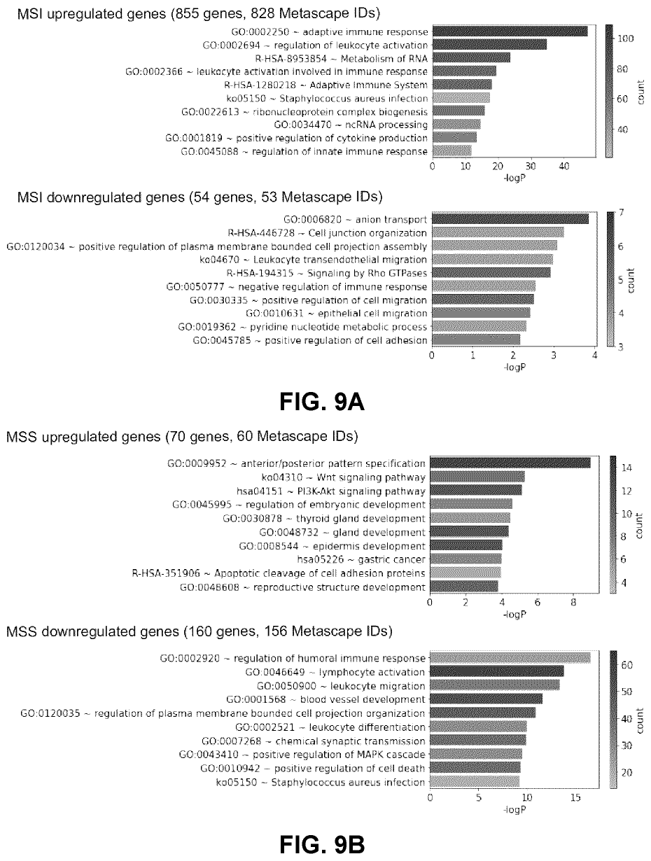

Principal component analysis of the top 500 varying genes between normal and tumor biopsy samples (FIG. 2A) or cell lines FIG. 9A) confirms their distinct transcriptomic profile. Accordingly, pathway and process enrichment analysis of both CRC-derived cell line and biopsy samples revealed a transcriptomic profile enriched in terms associated with their tumorigenic status. The most significantly up- and down-regulated GO terms are respectively linked to cell proliferation (FIG. 2B upper panel) and muscle phenotype and contractility (FIG. 2B lower panel). While the enrichment of GO terms related to proliferation and cell cycle is a general hallmark of cancer (50, 51), the downregulation of muscle-related pathways is inherent to CRC and results from the functional dichotomy between poorly differentiated tumor areas and highly contractile NAT. Among the tumor samples of the datasets used herein, it is then the MSI/MSS status which seems to account for inter-tumor transcriptomic differences (FIGS. 2A and 9A). While MSI samples tend to cluster tightly together, MSS tumors appear more dispersed and therefore transcriptionally more heterogeneous. Functionally, when analyzed separately, the MSS and MSI CRC samples are enriched in very different gene sets. When compared to their corresponding NAT, MSI tumors are characterized by a significant up-regulation of various immune-related GO terms (FIG. 9A) whereas MSS tumors are more associated with an increased expression of genes related to both Wnt and PI3K-Akt signaling (FIG. 9B). Although the link of these two signaling pathways with CRC is known (52), no reference could be found to support that their contribution in CRC may differ between MSS and MSI tumors.

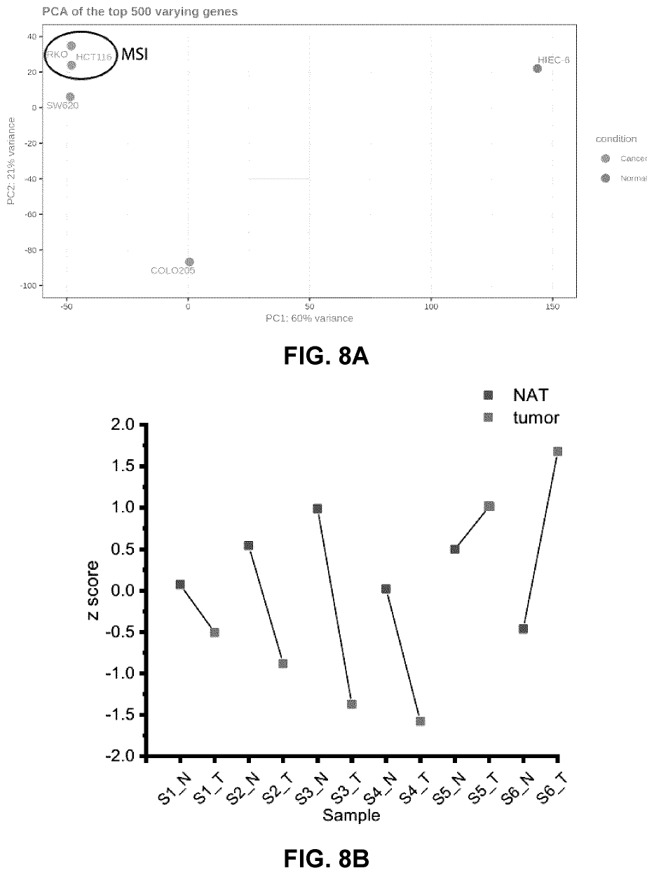

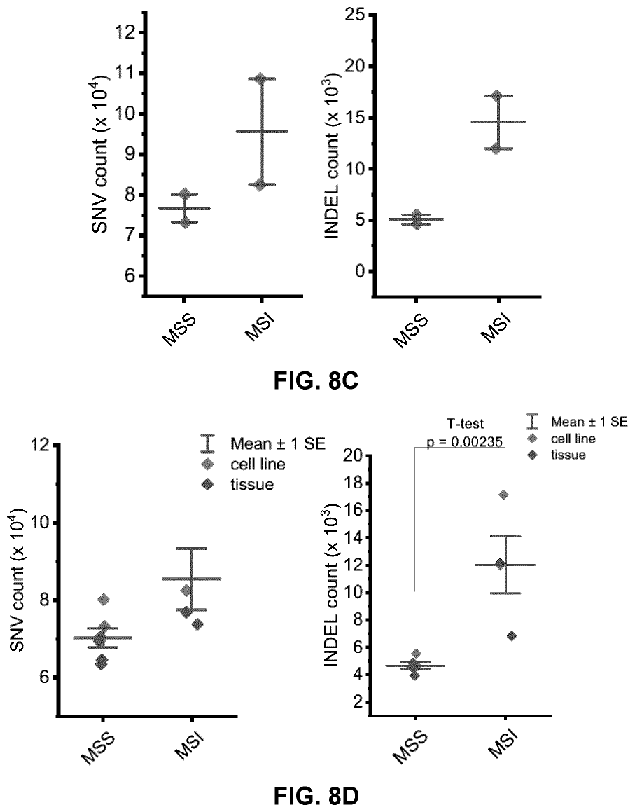

Next, the degree of immune infiltration of each sample was estimated via two independent approaches using the immune infiltration score from the ESTIMATE package (37) (FIG. 2C), and with an enrichment score for known tumor-infiltrating leukocyte (TIL) markers (53) based on a single-sample Gene Set Enrichment Analysis (ssGSEA) (54) (FIG. 8B). While all NAT samples presented similar levels of immune infiltration, MSI and MSS tumors were respectively characterized by increased and decreased immune infiltration scores (FIG. 2C and FIG. 8B). Such differences suggest that MSI tumors may be more immunogenic than their MSS homologs (48, 55-58).

Because TSAs can arise from a wide range of cancer-specific events/dysregulations (11, 12) and that the immunopeptidome contribution of each antigenic source varies significantly across malignancies (11), RNA sequencing data were also used to inform which TSA classes might be enriched in the samples. By looking at the genomic origin of the transcripts, it was observed that both the proportion and the absolute abundance of non-coding polyadenylated RNAs are significantly increased in tumors compared to NATs (FIG. 2D). While on average the absolute abundance increase is limited to 25%, the data suggests that the tumor-specific gain of non-coding transcripts could be higher in MSI tumors than in MSS. Although this comparison remains limited due to the low number of MSI samples (n=2), one could expect to identify a higher number of aeTSA deriving from non-coding transcripts in MSI sample than in MSS. Similarly, while all CRC samples presented comparable single nucleotide variant (SNV) burdens and are therefore expected to have similar numbers of mTSAs (FIG. 2E), the insertion/deletion (indel) burden was notably increased in MSI samples compared to MSS, an observation that is also noted for cell lines (FIG. 8C). In fact, considering both cell line and tissue samples together resulted in a statistically significant difference in the number of INDEL mutations between MSS and MSI samples (p=0.00235) (FIG. 8D). Because both MSI and INDEL accumulation result from defects in the DNA mismatch repair (MMR) pathway (59), one can hypothesize that the number of INDEL-derived TSAs (most likely frameshift-derived antigens) identified in a sample will be proportional to its MSI level.

### Example 4: Immunopeptidomic Analyses Highlight the Diversity of CRC Antigens

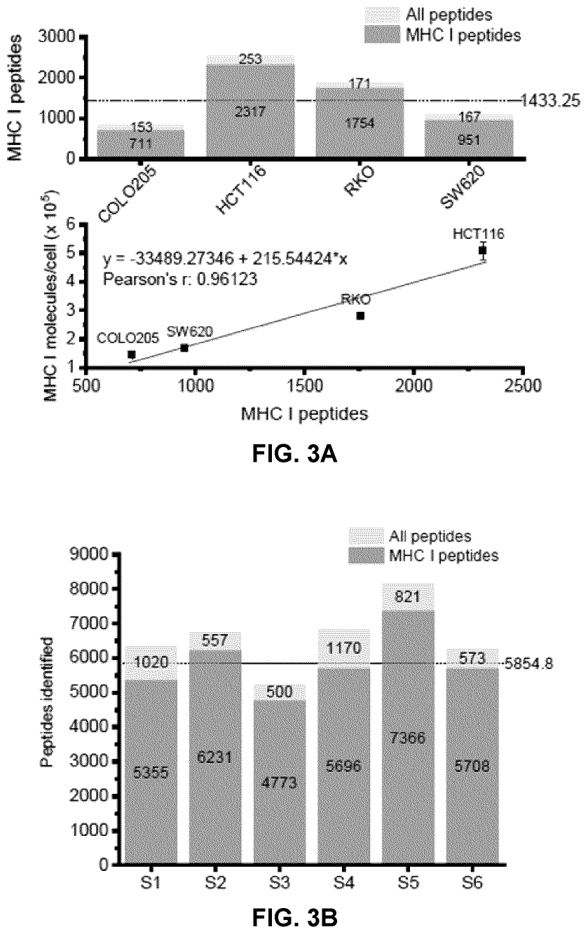

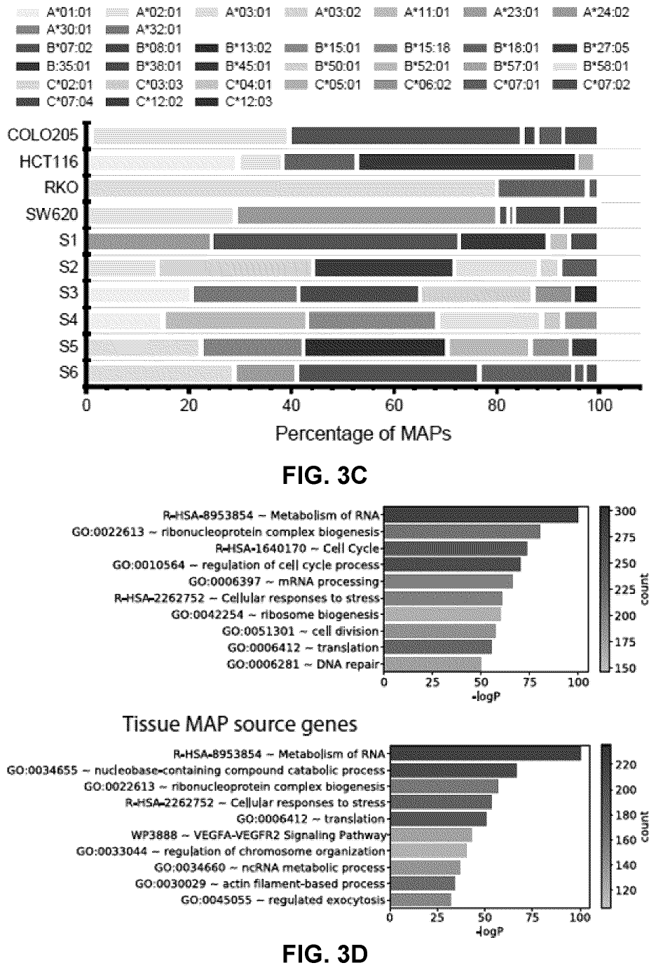

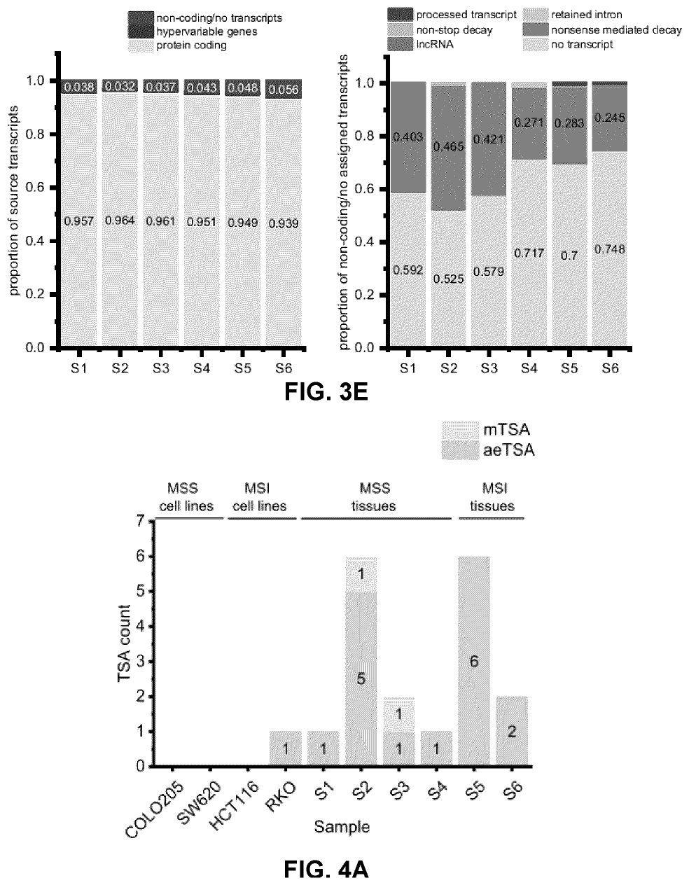

To elucidate the MHC I immunopeptidomes of CRC-derived cell lines, MAPs from 4 replicates of 2×10⁸ cells were immunoprecipitated for each line, and each replicate was derivatized with a separate TMT6plex channel (126, 127, 128, 129) for cell lines or with TMT10plex-126 and -127N for primary NAT and tissue samples, respectively. The four replicates of each cell line, and half of the respective NAT and tumor MAPs from each subject, were multiplexed and analyzed by LC-MS/MS. The median labeling efficiencies were 72.4% or 87.8% for cell lines and tissue samples, respectively. The lower efficiency of labeling in cell lines was ascribed to meager MAP yields. 5281 and 27 583 unique MAPs were identified in the cell line and tissue datasets, respectively, with a mean of 1433 unique MAPs per cell line and 5855 per tissue (FIG. 3A, upper panel, and FIG. 3B). While the identification varies between each line, the number of MAPs identified is strongly correlated with the abundance of MHC I molecules per cell (FIG. 3A, bottom panel; Pearson's r=0.96).

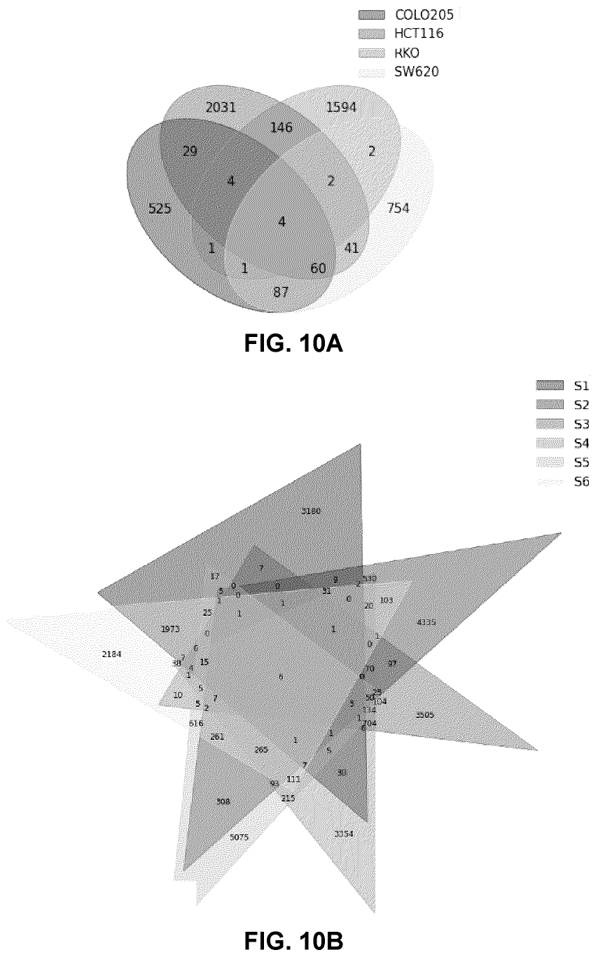

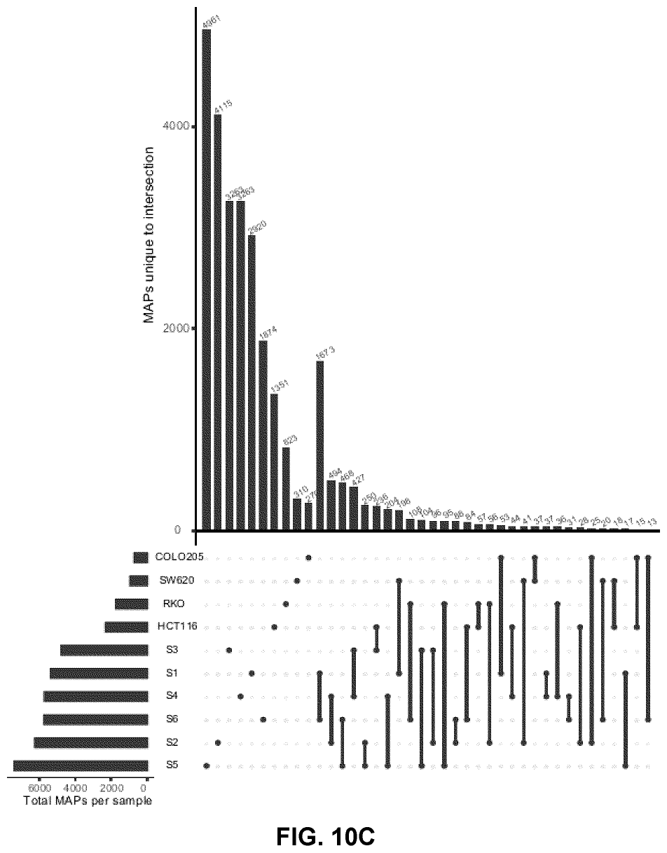

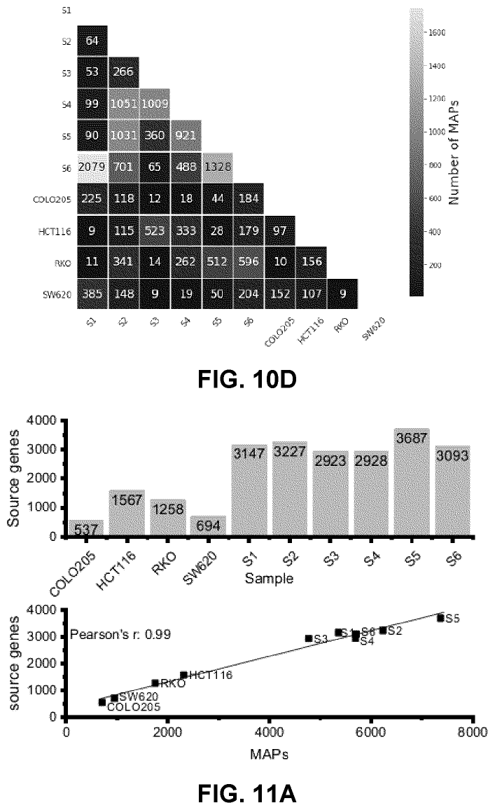

When taking the cell line and tissue samples together, a total of 30485 unique MAPs were identified. Within the MAP repertoire of each sample, 32-68% of the peptides are sample-specific, and even in comparing only cell line or primary samples, there are very few shared MAPs (FIG. 10A-B). This large proportion of unique MAPs can be attributed to the diversity of HLA alleles among the samples, which are a major factor influencing which peptides are able to be presented at the cell surface (FIG. 3C; FIG. 7). On average, the number of MAPs shared by any two cell lines or any two tissue samples is 59 or 640 MAPs, respectively. There are noteworthy outliers-tissue samples S1 and S6 shared 2079 MAPs (1673 of which are unique to these samples (FIG. 10C)), representing more than one third of their respective MHC I immunopeptidomes (FIG. 10D). The next-closest similarity in MAP repertoires between two tissues is 1328 MAPs shared by the two MSI tissues (S5 and S6), corresponding to 21% of their repertoires. The decreased MAP identification in cell lines makes these comparisons less striking. For example, HCT116 and RKO share the most MAPs, though these peptides represent less than 10% of their MAPs and is likely a feature of their larger peptide repertoires (FIG. 10C). In contrast, COLO205 and SW620 share 152 MAPs, nearly one fifth of their immunopeptidomes.

To contextualize these comparisons, the HLA alleles of the samples can again be considered. Out of the 2079 MAPs shared by S1 and S6, 1542 MAPs are predicted to bind the same allele in both samples, and that allele is HLA-B*07:02 in over 93% of these cases. Similarly, 136 out of 152 MAPs shared by COLO205 and SW620 are bound by the same allele, and that allele is HLA-A*02:01 in 113 of these cases. Thus, the MHC I immunopeptidomes of the samples is mainly influenced by the HLA repertoire.

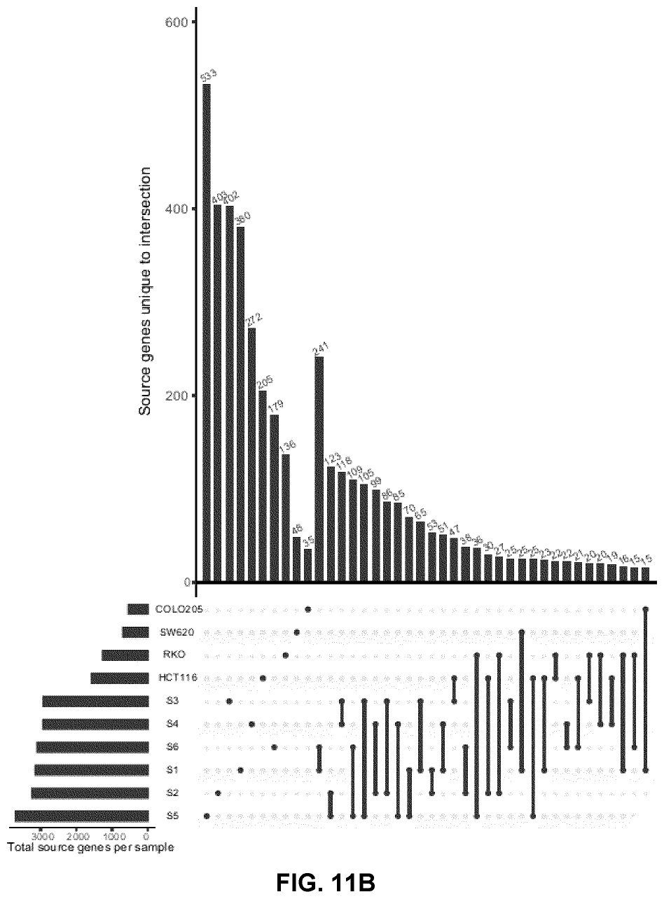

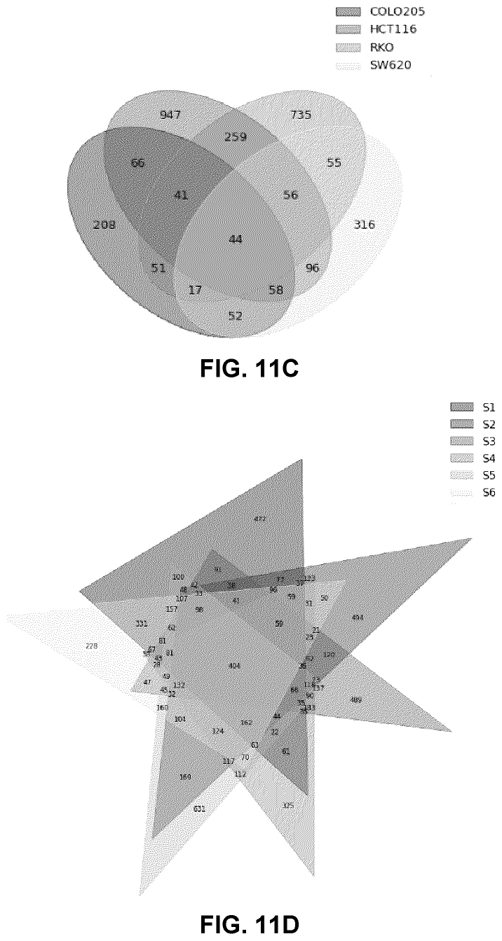

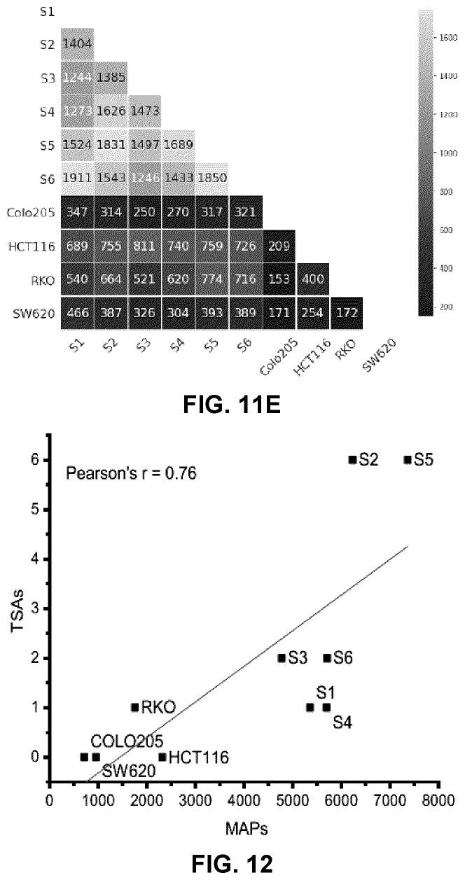

At the gene level, peptides derived from over 8000 unique source genes were identified, with an average of 1014 and 3168 source genes per cell line and tissue sample, respectively (FIG. 11A, upper panel). The number of source genes identified in each sample is highly correlated with the number of MAPs identified (FIG. 11A, lower panel). Roughly 6-14% of the source genes in a given immunopeptidome are sample-specific (FIG. 11B), which could be attributed to sample-specific biological features or it could reflect an imperfect sampling of the immunopeptidome (FIG. 11E). It is not expected to identify every MAP presented at the cell surface, and since a majority of source genes in each sample are attributable to only a single MAP, it is almost certain that additional source genes contribute to the MAP repertoire and are simply not detected. When comparing any two tissue samples, they have on average 48% source genes in common, while comparing any two cell lines results in an average of 24% genes being shared (FIGS. 11C-E). Thus, distinct cell lines appear to be less homogenous at source gene level. This likely reflects differences in sample composition, as the tissue samples have source genes derived from NAT, stroma, infiltrating cells, etc., while cell lines consist of only a single cell type. In addition, lower MHC I presentation of cell lines and the resulting decreased identification of MAPs means fewer source genes were sample, lower the likelihood of overlap. Regardless, all samples are more similar at the source gene level compared to the immunopeptidome level, and sample-specific MAPs are being derived from shared source genes.

To obtain an overview of the genomic origin of the MHC I immunopeptidome and investigate the shared nature of source genes, GO term analysis was performed on all the source genes identified in the cell lines as well as those identified in 4 or more tissues. Several common features between cell lines and tissues are detectable at the immunopeptidome level, including a significant enrichment of genes involved in RNA metabolism, ribonucleoprotein complex biogenesis, translation, and cellular responses to stress (FIG. 3D). Thus, despite the heterogeneity both between and among the cell lines and tissue samples, including the large diversity of HLA alleles which greatly impacts the peptide repertoire, and the low MAP identification in cell lines, there is significant similarity in terms of what genes are contributing to the MHC I immunopeptidome.

To investigate what proportion of MAPs from the tissue samples were from non-coding transcripts, it was first determined, for each peptide, the most abundant putative source transcript (Ensembl Annotation 99). For peptides from the cancer-specific database, the MCS were mapped onto the genome, and determined the most expressed transcript at that location (see ‘Quantification of MAP coding sequences in RNA-Seq data’ in Methods section). It was thus determined that on average, 95.3% of the MAPs from tissue samples were from protein coding transcripts (i.e. UTR or CDR) (FIG. 3E, left panel). Approximately 4.2% of peptides are from non-coding regions if the 2.8% of peptides deriving from unannotated RNA transcripts is included, as these peptides are likely coming from intergenic sequences. Approximately one-third of all noncoding MAPs (including those from unannotated transcripts) are derived from nonsense-mediated decay transcript products, while less than 1% of them are coming from lncRNA, nonstop decay products, retained introns, or processed transcripts (transcripts that do not contain open reading frames) (FIG. 3E, right panel).

### Example 5: Identification of Tumor-Specific and Tumor-Associated Antigens in CRC

Following the identification of over 30,000 unique MAPs, peptide coding sequences were filtered to select those overexpressed at least 10-fold in cancer and expressed <2rphm in pooled mTEC samples or matched NAT, for cell lines and primary samples, respectively. A recent immunopeptidomic study in acute myeloid leukemia (AML) demonstrated that MCSs with RPHM <8.55 have less than 5% probability to generate MAPs (18). The MAP coding sequences in RNA-data were then quantified, and only those that were expressed less than 8.55rphm in mTECs and other normal tissues (GTEx) were kept. Following manual validation of the remaining peptides, peptides were classified as aberrantly expressed TSAs (aeTSAs) if they were overexpressed at least 10-fold in tumor and were expressed <0.2kphm in mTECs (and NAT in the case of tissues). MAPs were classified as TAAs if they were also overexpressed at least 10-fold in cancer but their expression in mTEC and/or NAT was greater than 0.2kphm.

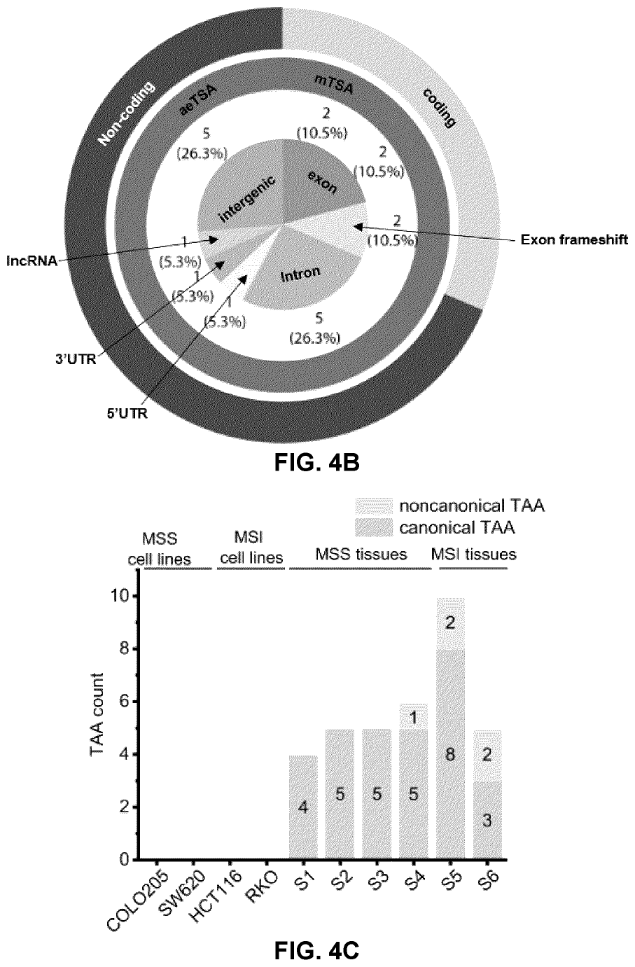

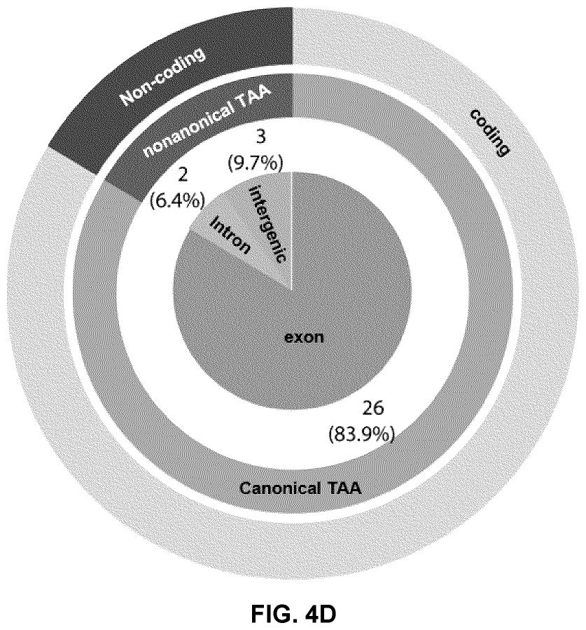

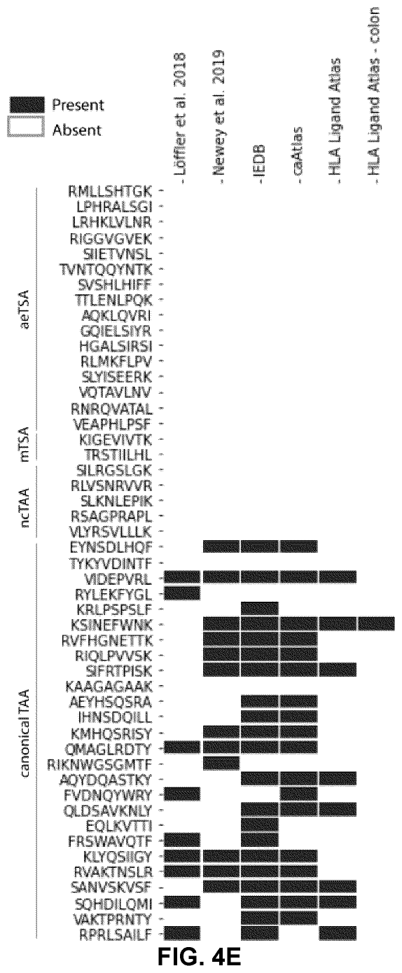

While the TSA identification in CRC-derived cell lines was relatively meager, possibly due in part to the low MAP identification, an average of 3 TSAs was identified per primary tissue sample (FIG. 4A). Overall, 1 putative TSA was identified in a CRC-derived cell line and 18 putative TSAs were identified in primary tissues, and the TSA yield from each sample was correlated with the number of MAPs identified (Pearson's r=0.76) (FIG. 12). Of these, approximately one-third were derived from coding regions, while the majority of the putative TSAs identified originated from non-coding regions (FIG. 4B). Among the TSAs from coding regions, two were from non-canonical reading frames, deriving from exon frameshift sequences, and another two were mutated TSAs identified in MSS tissues S2 and S3 (FIGS. 4A and 4B). Among the non-coding TSAs, all were aberrantly expressed, and a large proportion originates from intronic or intergenic regions, with a smaller number being derived from 5′ UTR, 3′ UTR, or lncRNAs (FIG. 4B). The sequences of nine aeTSAs (5 introns, 3 intergenic, 1 lncRNA) overlap ERE sequences (Table 4). Due to the ubiquitous nature of EREs, TSAs derived from aberrant ERE expression are potentially shared by tumors and have been shown to be immunogenic (61, 62). Of note, none of the putative TSAs were shared between multiple samples, even those with a high proportion of shared MAPs. However, two unique TSAs were identified in different tissues that were derived from the same transcript of COL11A1 (one exon frameshift and one 5′ UTR), which was recently shown to play a role in CRC development and prognosis (63). The majority of other TSA source genes have also been shown to be biologically relevant in CRC (Table 5).

TABLE 4
Characteristics of the TSAs and TAAs identified in the present studies

| Peptide | | | | | |
|---|---|---|---|---|---|
| sequence | | | | | |
| (SEQ ID NO:) | Sample | Class | Source | Gene Symbol | TranscriptID |
| RMLLSHTGK (1) | RKO | aeTSA | intergenic | none | none |
| LPHRALSGI (2) | S1 | aeTSA | intron | NKD1 (INHIBITOR | ENST00000268459.6 |
| | | | | OF WNT | |
| | | | | PATHWAY) | |
| GTNPTAAVK (3) | S2 | aeTSA | exon | HKDC1 | ENST00000354624.6 |
| LRHKLVLNR (4) | S2 | aeTSA | 3′ UTR | GRIN2B | ENST00000609686.3 |
| RIGGVGVEK (5) | S2 | aeTSA | exon frameshift | LY6G6F-LY6G6D | ENST00000503322.1 |
| | | | | readthrough | |
| SIIETVNSL (6) | S2 | aeTSA | intron | IPP | ENST00000396478.4 |
| TVNTQQYNTK | S2 | aeTSA | intron | CYP39A1 | ENST00000275016.3 |
| (7) | | | | | |
| SVSHLHIFF (8) | S3 | aeTSA | intergenic | none | none |
| TTLENLPQK (9) | S4 | aeTSA | exon frameshift | COL11A1 | ENST00000370096.9 |
| AQKLQVRI (10) | S5 | aeTSA | intergenic | none | none |
| GQIELSIYR (11) | S5 | aeTSA | intron | PATJ | ENST00000371158.6 |
| HGALSIRSI (12) | S5 | aeTSA | intron | DHP6 | ENST00000561411.1 |
| RLMKFLPV (13) | S5 | aeTSA | intergenic | none | none |
| SLYISEERK (14) | S5 | aeTSA | IncRNA | TRPC6 | ENST00000526713.1 |
| VQTAVLNV (15) | S5 | aeTSA | intergenic | none | none |
| RNRQVATAL | S6 | aeTSA | exon | SUCNR1 | ENST00000362032.6 |
| (16) | | | | | |
| VEAPHLPSF (17) | S6 | aeTSA | 5′ UTR | COL11A1 | ENST00000370096.9 |
| KIGEVIVTK (18) | S2 | mTSA | exon | HSPD1 | ENST00000388968.8 |
| TRSTIILHL (19) | S3 | mTSA | exon | PLK1 | ENST00000300093.9 |
| SILRGSLGK (20) | S4 | ncTAA | intergenic | none | none |
| RLVSNRVVR | S5 | ncTAA | intron | IDO2 | ENST00000518237.6 |
| (21) | | | | | |
| SLKNLEPIK (22) | S5 | ncTAA | intergenic | none | none |
| RSAGPRAPL | S6 | ncTAA | intergenic | none | none |
| (23) | | | (antisense of | | |
| | | | transcript) | | |
| VLYRSVLLLK | S6 | ncTAA | intron | FANCA | ENST00000389301.8 |
| (24) | | | | | |
| EYNSDLHQF | S1 | TAA | exon | BUB1 | ENST00000302759.11 |
| (25) | | | | | |
| TYKYVDINTF | S1 | TAA | exon | MMP12 | ENST00000571244.3 |
| (26) | | | | | |
| VIDEPVRL (27) | S1 | TAA | exon | MKI67 | ENST00000368653.7 |
| RYLEKFYGL (28) | S1, S6 | TAA | exon | MMP12 | ENST00000571244.3 |
| KRLPSPSLF (29) | S2 | TAA | exon | MGAM2 | ENST00000550469.6 |
| KSINEFWNK (30) | S2 | TAA | exon | SPC25 | ENST00000282074.7 |
| RVFHGNETTK | S2 | TAA | exon | CENPE | ENST00000265148.8 |
| (31) | | | | | |
| RIQLPVVSK (32) | S2, S4 | TAA | exon | MKI67 | ENST00000368653.7 |
| SIFRTPISK (33) | S2, S4 | TAA | exon | ASPM | ENST00000367409.9 |
| KAAGAGAAK | S2, S5, | TAA | exon | HI-5 | ENST00000331442.4 |
| (34) | S6 | | | | |
| AEYHSQSRA | S3 | TAA | exon | ASPM | ENST00000367409.9 |
| (35) | | | | | |
| IHNSDQILL (36) | S3 | TAA | exon | MACC1 | ENST00000400331.10 |
| KMHQSRISY (37) | S3 | TAA | exon | ASPM | ENST00000367409.9 |
| QMAGLRDTY | S3 | TAA | exon | SPC25 | ENST00000282074.7 |
| (38) | | | | | |
| RIKNWGSGMTF | S3 | TAA | exon | NOS2 | ENST00000313735.11 |
| (39) | | | | | |
| AQYDQASTKY | S4 | TAA | exon | CENPF | ENST00000366955.8 |
| (40 | | | | | |
| FVDNQYWRY | S4 | TAA | exon | MMP12 | ENST00000571244.3 |
| (41) | | | | | |
| QLDSAVKNLY | S4 | TAA | exon | ZNF215 | ENST00000414517.6 |
| (42) | | | | | |
| EQLKVTTI (43) | S5 | TAA | exon | MCM10 | ENST00000378714.7 |
| FRSWAVQTF | S5 | TAA | exon | NOS2 | ENST00000313735.11 |
| (44) | | | | | |
| KLYQSIIGY (45) | S5 | TAA | exon | DIAPH3 | ENST00000400324.9 |
| SANVSKVSF (46) | S5 | TAA | exon | ASPM | ENST00000367409.9 |
| SQHDILQMI (47) | S5 | TAA | exon | MKI67 | ENST00000368653.7 |
| VAKTPRNTY (48) | S5 | TAA | exon | MKI67 | ENST00000368654.8 |
| RPRLSAILF (49) | S6 | TAA | exon | DIAPH3 | ENST00000400324.9 |
| RVAKTNSLR (50) | S5 | TAA | exon | CDCA8 | ENST00000327331.2 |

| Peptide | | ERE | | | Predicted HLA |
|---|---|---|---|---|---|
| sequence | GeneID | overlap | Location | Strand | allele |
| RMLLSHTGK (1) | none | none | chr8: 127209405- | (−) | HLA-A*03:01 |
| | | | 127209431 | | |
| LPHRALSGI (2) | ENSG00000140807 | Antisense | chr16: 50564443- | (+) | HLA-B*07:02 |
| | | miRc | 50564469 | | |
| | | overlap | | | |
| GTNPTAAVK (3) | ENSG00000156510 | none | chr10: 69227303- | (+) | HLA-A*03:02 |
| | | | 69227329 | | |
| LRHKLVLNR (4) | ENSG00000273079 | none | chr12: 13549419- | (−) | HLA-B*27:05 |
| | | | 13549445 | | |
| RIGGVGVEK (5) | ENSG00000250641 | none | chr6: 31717798- | (+) | HLA-A*03:02 |
| | | | 31717824 | | |
| SIIETVNSL (6) | ENSG00000197429 | L1MEf | chr1: 45713038- | (−) | HLA-A*02:01 |
| | | overlap | 45713064 | | |
| TVNTQQYNTK | ENSG00000146233 | L1MD1 | chr6: 46644986- | (−) | HLA-A*03:02 |
| (7) | | overlap | 46645015 | | |
| SVSHLHIFF (8) | none | antisense | chr17: 65462740- | (+) | HLA-A*32:01 |
| | | L1MB3 | 65462766 | | |
| | | overlap | | | |
| TTLENLPQK (9) | ENSG00000060718 | none | chr1: 103078698- | (−) | HLA-A*11:01 |
| | | | 103078724 | | |
| AQKLQVRI (10) | none | none | chr3: 29988327- | (−) | HLA-B*52:01 |
| | | | 29988304 | | |
| GQIELSIYR (11) | ENSG00000132849 | none | chr1: 61808366- | (+) | HLA-A*03:01 |
| | | | 61808392 | | |
| HGALSIRSI (12) | ENSG00000134146 | L2a | chr15: 35525008- | (−) | HLA-B*52:01 |
| | | overlap | 35525034 | | |
| RLMKFLPV (13) | none | none | chr2: 170700443- | (−) | HLA-B*13:02 |
| | | | 170700466 | | |
| SLYISEERK (14) | ENSG00000137672 | LIME1 | chr11: 101656906- | (−) | HLA-A*03:01 |
| | | overlap | 101656932 | | |
| VQTAVLNV (15) | none | none | chr4: 93906720- | (−) | HLA-B*52:01 |
| | | | 93906697 | | |
| RNRQVATAL | ENSG00000198829 | none | chr3: 151881192- | (+) | HLA-B*07:02 |
| (16) | | | 151881218 | | |
| VEAPHLPSF | ENSG00000060718 | none | chr1: 103108448- | (−) | HLA-B*18:01 |
| (17) | | | 103108474 | | |
| KIGEVIVTK (18) | ENSG00000144381 | none | chr2: 197489161- | (−) | HLA-A*03:02 |
| | | | 197489188 | | |
| TRSTIILHL (19) | ENSG00000166851 | none | chr16: 23689617- | (+) | HLA-C*06:02 |
| | | | 23689643 | | |
| SILRGSLGK | none | CR1_Mam | chr7: 140260497- | (−) | HLA-A*11:01 |
| (20) | | overlap | 140260523 | | |
| RLVSNRVVR | ENSG00000131203 | none | chr8: 39917585- | (+) | HLA-A*30:01 |
| (21) | | | 39917611 | | |
| SLKNLEPIK (22) | none | MSTA | chr15: 97429857- | (+) | HLA-A*30:01 |
| | | overlap | 97429883 | | |
| RSAGPRAPL | none | none | chr1: 145096230- | (−) | HLA-B*07:02 |
| (23) | | | 145096256 | | |
| VLYRSVLLLK | ENSG00000187741 | none | chr16: 89798385- | (−) | HLA-A*03:01 |
| (24) | | | 89798414 | | |
| EYNSDLHQF | ENSG00000169679 | none | chr2: 110672828- | (−) | HLA-A*24:02 |
| (25) | | | 110672854 | | |
| TYKYVDINTF | ENSG00000262406 | none | chr11: 102867951- | (−) | HLA-A*24:02 |
| (26 | | | 102867980 | | |
| VIDEPVRL (27) | ENSG00000148773 | none | chr10: 128122922- | (−) | HLA-C*04:01 |
| | | | 128122945 | | |
| RYLEKFYGL | ENSG00000262406 | none | chr11: 102873086- | (−) | HLA-A*24:02, |
| (28) | | | 102873112 | | HLA-A*23:01 |
| KRLPSPSLF | ENSG00000257743 | none | chr7: 142131535- | (+) | HLA-C*07:01 |
| (29) | | | 142131561 | | |
| KSINEFWNK | ENSG00000152253 | none | chr2: 168889463- | (−) | HLA-A*03:02 |
| (30) | | | 168889489 | | |
| RVFHGNETTK | ENSG00000138778 | none | chr4: 103196221- | (−) | HLA-A*03:02 |
| (31) | | | 103196250 | | |
| RIQLPVVSK | ENSG00000148773 | none | chr10: 128123124- | (−) | HLA-A*03:02, |
| (32) | | | 128123150 | | HLA-A*11:01 |
| SIFRTPISK (33) | ENSG00000066279 | none | chr1: 197139840- | (−) | HLA-A*03:02, |
| | | | 197139866 | | HLA-A*11:01 |
| KAAGAGAAK | ENSG00000184357 | none | chr6: 27867425- | (−) | HLA-A*03:02, |
| (34) | | | 27867451 | | HLA-A*30:01, |
| | | | | | HLA-A*03:01 |
| AEYHSQSRA | ENSG00000066279 | none | chr1: 197100800- | (−) | HLA-B*50:01 |
| (35) | | | 197100826 | | |
| IHNSDQILL (36) | ENSG00000183742 | none | chr7: 20159866- | (−) | HLA-B*38:01 |
| | | | 20159892 | | |
| KMHQSRISY | ENSG00000066279 | none | chr1: 197102843- | (−) | HLA-A*32:01 |
| (37) | | | 197102869 | | |
| QMAGLRDTY | ENSG00000152253 | none | chr2: 168889412- | (−) | HLA-A*01:01 |
| (38) | | | 168889438 | | |
| RIKNWGSGMTF | ENSG00000007171 | none | chr17: 27788839- | (−) | HLA-A*32:01 |
| (39) | | | 27788871 | | |
| AQYDQASTKY | ENSG00000117724 | none | chr1: 214622255- | (+) | HLA-B*15:01 |
| (40) | | | 214629048 | | |
| FVDNQYWRY | ENSG00000262406 | none | chr11: 102864249- | (−) | HLA-A*01:01 |
| (41) | | | 102865798 | | |
| QLDSAVKNLY | ENSG00000149054 | none | chr11: 6943137- | (+) | HLA-A*01:01 |
| (42) | | | 6943166 | | |
| EQLKVTTI (43) | ENSG00000065328 | none | chr10: 13172417- | (+) | HLA-B*52:01 |
| | | | 13172440 | | |
| FRSWAVQTF | ENSG00000007171 | none | chr17: 27768980- | (−) | HLA-C*06:02 |
| (44) | | | 27769006 | | |
| KLYQSIIGY (45) | ENSG00000139734 | none | chr13: 59833182- | (−) | HLA-A*03:01 |
| | | | 59833208 | | |
| SANVSKVSF | ENSG00000066279 | none | chr1: 197143439- | (−) | HLA-C*12:02 |
| (46) | | | 197143465 | | |
| SQHDILQMI | ENSG00000148773 | none | chr10: 128112176- | (−) | HLA-B*13:02 |
| (47) | | | 128112202 | | |
| VAKTPRNTY | ENSG00000148773 | none | chr10: 128109293- | (−) | HLA-C*12:02 |
| (48) | | | 128109319 | | |
| RPRLSAILF (49) | ENSG00000139734 | none | chr13: 59879421- | (−) | HLA-B*07:02 |
| | | | 59879447 | | |
| RVAKTNSLR | ENSG00000134690 | none | chr1: 37692715- | (+) | HLA-A*03:01 |
| (50) | | | 37692741 | | |

TABLE 5
Biological relevance of TSA source genes in CRC.

| Source gene | Reference | Biological relevance in CRC |
|---|---|---|
| NKD1 - Naked cuticle homolog | PMID: 25446263; | Negative feedback regulator of Wnt pathway, |
| 1 | PMID: 19956716 | intestinal tumor marker in mice; mutations in |
| | | NKD1 alter Wnt signaling |
| ANTXR1 - Anthrax toxin | PMID: 31977138; | Upregulated and associated with poor |
| receptor 1 | PMID: 22340594 | prognosis in gastric cancer, activates mTOR |
| | | pathway; ANTRXR1 blockade prevents |
| | | angiogenesis in CRC |
| HKDC1 - Hexokinase domain- | PMID: 30005951 | HKDC1 contributes to increased metabolism, |
| containing protein 1 | | proliferation, and metastasis of CRC cells |
| GRIN2B - Glutamate | PMID: 27243824 | Identified as non-driver hub gene involved in |
| ionotropic receptor NMDA type | | progression to stage II CRC |
| subunit 2B | | |
| LY6G6F-LY6G6D readthrough - | PMID: 26894861 | LY6G6D/F overexpressed in CRC, potential |
| Lymphocyte antigen 6 family | | cell surface marker |
| member G6F and G6D | | |
| IPP (KLHL27) - Intracisternal A | Human Protein | Favorable prognostic marker in colorectal |
| particle-promoted polypeptide | Atlas (PMID: | cancer; unfavorable in renal and liver cancers |
| | 28818916) | |
| CYP39A1 - ytochrome P450, | PMID: 27341022 | Expression is increased in CRC with poor |
| family 39, subfamily A, | | prognosis |
| polypeptide 1 | | |
| COL11A1 - Collagen type XI | PMID: 33597969 | Upregulated in CRC (mRNA), marker of poor |
| alpha 1 | | prognosis, role in CRC development |
| PATJ - PALS1-associated tight | | No known association |
| junction protein | | |
| DPH6 - Diphthamine | | No known association |
| biosynthesis 6 | | |
| TRPC6 - Rransient receptor | PMID: 26422106 | mRNA expression of TRPC6 lower in CRC |
| potential cation channel | | than in normal tissue, may contribute to |
| subfamily C member 6 | | tumorigenesis |
| SUCNR1 - Succinate receptor | PMID: 32365557 | SUCNR 1 activation induces Wnt ligand |
| 1 | | expression and activates WNT signaling and |
| | | EMT in a CRC-derived cell line |
| POF1B - Premature ovarian | PMID: 29484395; | Possible involvement with ATP5J functions in |
| failure protein 1B | PMID: 25084053 | CRC cell migration; regulates adhesion in |
| | | intestinal cell lines |
| HSPD1 - Heat shock protein | PMID: 28261350; | Differentially expressed in CRC, potential |
| family D (Hsp60) member 1 | PMID: 29246022 | biomarker for diagnosis; Exosomal HSPD1 |
| | | identified as putative diagnostic and prognostic |
| | | biomarker in CRC |
| PLK1 - Serine/threonine- | PMID: 22648245 | Overexpressed in CRC, associated with |
| protein kinase PLK1/polo-like | | metastasis and invasion |
| kinase 1 | | |
| MATR3 - Matrin 3 | PMID: 28580901 | MATR3 was shown to participate in pro- |
| | | survival activity of CRC-derived cells in |
| | | response to DNA damage, through interactions |
| | | with PINCR IncRNA and p53 |

While the primary objective was to identify putative TSAs in CRC, an average of 6.33 TAAs were also identified in the CRC tissue samples, though none were identified in the CRC-derived cell lines (FIG. 4C). In contrast to the primarily non-coding putative TSAs, the majority of the TAAs identified were from canonical, coding exonic sequences, with only a small number being derived from introns, intergenic sequences, or lncRNAs (FIG. 4D). Two non-canonical TAAs were overlapped by ERE sequences (Table 4). Of note, 4 separate TAAs were identified in more than one sample, with one TAA being identified in 3 tissues. These repeated TAAs were all derived from canonical exons, with source transcripts deriving from ASPM, MKI67, DIAPH3, MMP12, NOS2, and SPC25, all of which have documented associations with cancer (Table 6).

TABLE 6
Biological relevance of TAA source genes in CRC.

| Gene symbol | Reference | Biological relevance in CRC |
|---|---|---|
| MMP12 - Matrix | PMID: 27431388 | Overexpressed in CRC compared to control, |
| metallopeptidase 12 | | negative prognostic marker in CRC |
| MKI67 - Marker of | PMID: 26281861; | Favorable prognostic marker in CRC , IHC staining |
| proliferation Ki-67 | PMID: 27855388; | (2016); favorable prognostic marker in stage III and |
| | PMID: 30727976 | IV CRC, IHC staining (2016); poor prognostic |
| | | marker in CRC based on database meta-analysis |
| | | (2019 |
| BUB1 - Mitotic spindle | PMID: 23747338; | Mutations in BUB1 linked to early onset CRC; |
| checkpoint kinase | PMID: 11782350 | inactivation may drive metastasis and progression |
| | | in CRC |
| DIAPH3 - Diaphanous | Human Protein | DIAPH3 is prognostic, high expression is favorable |
| related formin 3 | Atlas (PMID: | in colorectal cancer |
| | 28818916) | |
| MGAM2 - Maltase | PMID: 30996822 | Expressed in GI cancers (TCGA data) |
| glucoamylase 2 | | |
| SPC25 (kinetochore | PMID: 32351050; | Highly expressed in CRC (among other cancers); |
| protein) | Human Protein | unfavorable prognostic marker in liver cancer, |
| | Atlas (PMID: | endometrial cancer, and lung cancer |
| | 28818916) | |
| CENPE - Centromere- | | No known association |
| associated protein E | | |
| ASPM -Abnormal spindle | PMID: 31966766; | Overexpressed in CRC; suggested to be |
| microcephaly associated | Human Protein | unfavorable prognostic marker (involved in mitosis, |
| | Atlas (PMID: | cell cycle, tumorigenesis); known to be unfavorable |
| | 28818916) | prognostic marker in liver, lung, endometrial, |
| | | pancreatic cancers |
| HI-5 - H1.5 linker histone, | PMID: 16959974 | Frequently mutated in CRC |
| cluster member | | |
| MACC1 - Metastasis- | PMID: 27424982; | Promotes growth and metastasis of colorectal |
| associated in colon cancer | PMID: 25003996 | cancer; associated with carcinogenesis through B- |
| 1 | | catenin signaling and EMT transition |
| NOS2 - Nitric oxide | Human Protein | Cancer enhanced (colorectal cancer); RNA data |
| synthase 2 | Atlas (PMID: | |
| | 28818916) | |
| CENPF - Centromere | PMID: 30550624 | phosphorylation changes associated w CRC |
| protein F | | malignancy; unfavorable prognostic marker in other |
| | | cancers (liver, renal, etc.; human protein atlas) |
| ZNF215 - Zinc finger | Human Protein | Cytoplasmic expression in subsets of immune cells, |
| protein 215 | Atlas (PMID: | most abundant in gastrointestinal tract and |
| | 28818916) | lymphoid tissues (protein data) |
| MCM10 - | PMID: 32597491 | Decreased mRNA expression in colon and rectal |
| Minichromosome | | adenocarcinoma samples compared to normal |
| maintenance 10 | | tissues |
| replication initiation factor | | |
| CDCA8 - Cell division | PMID: 25260804 | overexpressed in CRC, associated with cancer |
| cycle associated 8 | | progression |
| IDO2 - Indoleamine 2,3- | PMID: 18418598; | Upregulated expression in CRC |
| dioxygenase 2 | | |
| UNG - Uracil DNA | PMID: 17029639 | Frequent germline mutations in patients with CRC |
| glycosylase | | |
| FANCA - Fanconi anemia | PMID: 27165003; | Fanconi anemia predisposes certain cancers; |
| group A protein | PMID: 21286667 | genes in FA pathway participate in CRC |
| | | pathogenesis (involved in HR repair) |

It was initially expected to identify an above average number of both TSAs and TAAs in MSI tissues. This was the case in S5, however the same was not true for the other MSI tissue (FIGS. 4A and 4C). This could be due to S6 having a lower ‘degree’ of instability, as reflected in the MSIsensor results (Table 3). Further, the sample that had the highest number of identified TSAs was S2, an MSS tissue. Thus, the yield of TSAs and TAAs per sample seems to be irrespective of MSI status and may be due to other unique biological features of the tumor outside the scope of this study.

To determine if any of the putative TSAs or TAAs have been previously identified, it was verified if the peptide sequences were reported in the Immune Epitope Database (IEDB), the HLA Ligand Atlas (64), and 2 previous publications that sought to identify tumor antigens in CRC from Löffler et al. 2018 (8) and Newey et al. 2019 (15). Of note, none of the putative aeTSAs, mTSAs, or non-canonical TAAs have been previously reported in any of these resources. Of the 26 putative canonical TAAs identified, 24 of them were reported either in IEDB, Löffler et al 2018 (PXD009602), Newey et al 2019 (PXD014017), or some combination of the three (FIG. 4E). Eight of these were also reported in HLA Ligand Atlas, with one of them specifically being documented in healthy colon tissue. Interestingly, none of the TAAs previously identified in these earlier publications were reported as tumor antigens, and, conversely, six of the 12 tumor antigens of interest reported in Loffler et al. were also identified in the immunopeptidomes of the present study, though they did not pass the thresholds established in the identification pipeline to be considered TSAs or TAAs, most often due to high expression in NAT (Table 7A). Thus, novel colorectal cancer TSAs that derive primarily from non-coding regions, as well as a selection of mainly coding TAAs, some of which have been previously reported as MAPs but not in the context of their biological relevance as TAAs, have been identified in the present study. Tables 7B and 7C depict the TSAs and TAAs identified in the present studies, respectively.

TABLE 7A
Justifications for exclusion of Loffler et al. 2018 tumor antigens.

| | Loffler et al. | Loffler et al. | | |
|---|---|---|---|---|
| Peptide | 2018 | 2018 HLA | | Reason for |
| (SEQ ID NO) | classification | restriction | Sample | exclusion |
| RLASRPLLL | Vaccine | B*07 | S5 | FC < 10 |
| (101) | candidate | | | between cancer |
| | | | | and NAT |
| YRNSYEIEY | Vaccine | C*07 | S1, S2, | MCS |
| (102) | candidate | | S6 | expression > 2 |
| | | | | KPHM in NAT |
| APTPARPVL | Vaccine | B*07 | S1 | MCS |
| (103) | candidate | | | expression 8.55 |
| | requiring | | | RPHM in mTEC |
| | further | | | |
| | validation | | | |
| RLAEPSQMLK | Vaccine | A*03 | S6 | MCS |
| (104) | candidate | | | expression > 2 |
| | requiring | | | KPHM in NAT |
| | further | | | |
| | validation | | | |
| SPKATGVFTTL | Vaccine | B*07 | S1, S6 | MCS |
| (105) | candidate | | | expression > 2 |
| | requiring | | | KPHM in NAT |
| | further | | | |
| | validation | | | |
| SVLTQPPSV | Vaccine | A*02 | S2 | MCS |
| (106) | candidate | | | expression > 2 |
| | of unclear | | | KPHM in NAT |
| | evidence | | | |

TABLE 7B
TSAs identified in the present studies

| Sequences | SEQ ID | Predicted HLA | |
|---|---|---|---|
| (amino acids and nucleotides) | NO: | allele¹ | Status² |
| RMLLSHTGK | 1 | HLA-A*03:01 | Novel |
| AGGATGTTACTGAGCCACACAGGAAAA | 51 | | |
| LPHRALSGI | 2 | HLA-B*07:02 | Novel |
| CTTCCTCACAGGGCTTTGTCAGGCATT | 52 | | |
| GTNPTAAVK | 3 | HLA-A*03:02 | Novel |
| GGCACCAACCCCACGGCTGCAGTGAAG | 53 | | |
| LRHKLVLNR | 4 | HLA-B*27:05 | Novel |
| TTAAGGCATAAGTTAGTTCTGAACAGA | 54 | | |
| RIGGVGVEK | 5 | HLA-A*03:02 | Novel |
| CGGATAGGGGGAGTAGGAGTAGAGAAG | 55 | | |
| SIIETVNSL | 6 | HLA-A*02:01 | Novel |
| TCTATAATAGAAACTGTAAATTCTCTT | 56 | | |
| TVNTQQYNTK | 7 | HLA-A*03:02 | Novel |
| ACTGTCAACACTCAACAATATAATACAAAA | 57 | | |
| SVSHLHIFF | 8 | HLA-A*32:01 | Novel |
| TCTGTTAGCCATTTGCATATCTTCTTT | 58 | | |
| TTLENLPQK | 9 | HLA-A*11:01 | Novel |
| ACCACACTGGAAAACCTGCCCCAGAAG | 59 | | |
| AQKLQVRI | 10 | HLA-B*52:01 | Novel |
| GCACAAAAATTGCAAGTGAGGATT | 60 | | |
| GQIELSIYR | 11 | HLA-A*03:01 | Novel |
| GGTCAAATAGAGTTATCAATATATAGG | 61 | | |
| HGALSIRSI | 12 | HLA-B*52:01 | Novel |
| CATGGTGCCCTGTCCATAAGAAGTATT | 62 | | |
| RLMKFLPV | 13 | HLA-B*13:02 | Novel |
| AGACTGATGAAATTCCTTCCAGTT | 63 | | |
| SLYISEERK | 14 | HLA-A*03:01 | Novel |
| TCTCTGTATATCTCAGAAGAAAGGAAA | 64 | | |
| VQTAVLNV | 15 | HLA-B*52:01 | Novel |
| GTTCAAACTGCTGTTTTAAATGTG | 65 | | |
| KIGEVIVTK | 16 | HLA-A*03:02 | Novel |
| AAAATTGGAGAGGTCATTGTGACCAAA | 66 | | |
| TRSTIILHL | 17 | HLA-C*06:02 | Novel |
| ACCCGCAGCACCATCATCCTGCACCTC | 67 | | |
| RNRQVATAL | 24 | HLA-B*07:02 | Previously reported |
| AGGAATAGGCAGGTTGCTACTGCTCTG | 74 | | in US2022/0002374 |
| VEAPHLPSF | 25 | HLA-B*18:01 | Previously reported |
| GTGGAAGCCCCTCATCTGCCTTCATTC | 75 | | in WO2020/131586 |

TABLE 7C
TAAs identified in the present studies

| | SEQ ID | Predicted | |
|---|---|---|---|
| Sequence | NO: | HLA allele | Status |
| SILRGSLGK | 18 | HLA-A*11:01 | Novel |
| TCGATTTTACGTGGATCTCTAGGGAAA | 68 | | |
| RLVSNRVVR | 19 | HLA-A*30:01 | Novel |
| AGATTGGTTTCAAATAGAGTAGTCAGG | 69 | | |
| SLKNLEPIK | 20 | HLA-A*30:01 | Novel |
| AGCCTGAAAAACCTTGAGCCAATTAAA | 70 | | |
| RSAGPRAPL | 21 | HLA-B*07:02 | Novel |
| CGCTCTGCGGGTCCG | 71 | | |
| CGGGCCCCTCTC | | | |
| VLYRSVLLLK | 22 | HLA-A*03:01 | Novel |
| GTGTTGTACCGTTCG | 72 | | |
| GTACTTTTACTGAAA | | | |
| KAAGAGAAK | 23 | HLA-A*03:02, HLA- | Novel |
| AAGGCTGCCGGCGCC | 73 | A*30:01, HLA-A*03:01 | |
| GGCGCTGCTAAG | | | |
| EYNSDLHQF | 26 | HLA-A*24:02 | Previously reported in |
| GAGTACAACAGTGACCTCCATCAATTT | 76 | | IEDB and PXD014017² |
| TYKYVDINTF | 27 | HLA-A*24:02 | Previously reported in |
| ACCTACAAATATGTTG | 77 | | US2022/0175898A1 |
| ACATCAACACATTT | | | |
| VIDEPVRL | 28 | HLA-C*04:01 | Previously reported in |
| GTTATTGATGAGCCTGTACGGCTA | 78 | | IEDB, PXD009602, |
| | | | PXD014017, HLA |
| | | | Ligand Atlas² |
| RYLEKFYGL | 29 | HLA-A*24:02, HLA- | Previously reported in |
| AGATACTTAGAAAAATTTTATGGCCTT | 79 | A*23:01 | PXD009602² |
| KRLPSPSLF | 30 | HLA-C*07:01 | Previously reported in |
| AAAAGGTTGCCATCACCATCTCTGTTT | 80 | | PXD014017² |
| KSINEFWNK | 31 | HLA-A*03:02 | Previously reported in |
| AAAAGCATAAATGAATTTTGGAATAAA | 81 | | IEDB, PXD014017, |
| | | | and HLA Ligand Atlas² |
| RVFHGNETTK | 32 | HLA-A*03:02 | Previously reported in |
| CGTGTCTTTCATGGT | 82 | | IEDB and PXD014017² |
| AATGAAACTACCAAA | | | |
| RIQLPVVSK | 33 | HLA-A*03:02, HLA- | Previously reported in |
| CGTATCCAGCTTCCTGTTGTGTCAAAA | 83 | A*11:01 | IEDB and PXD014017² |
| SIFRTPISK | 34 | HLA-A*03:02, HLA- | Previously reported in |
| TCAATATTCAGAACTCCAATTTCTAAA | 84 | A*11:01 | IEDB and PXD014017² |
| AEYHSQSRA | 35 | HLA-B*50:01 | Previously reported in |
| GCAGAGTATCATTCTCAAAGTAGGGCT | 85 | | IEDB² |
| IHNSDQILL | 36 | HLA-B*38:01 | Previously reported in |
| ATACATAACTCTGACCAGATCCTACTA | 86 | | IEDB² |
| KMHQSRISY | 37 | HLA-A*32:01 | Previously reported in |
| AAAATGCATCAGTCAAGAATAAGTTAC | 87 | | IEDB and PXD014017² |
| QMAGLRDTY | 38 | HLA-A*01:01 | Previously reported in |
| CAGATGGCGGGACTAAGAGATACCTAC | 88 | | IEDB, PXD009602 and |
| | | | PXD014017² |
| RIKNWGSGMTF | 39 | HLA-A*32:01 | Previously reported in |
| AGGATCAAAAACTGGGGCAGC | 89 | | PXD014017² |
| GGGATGACTTTC | | | |
| AQYDQASTKY | 40 | HLA-B*15:01 | Previously reported in |
| GCACAATACGACCAG | 90 | | IEDB and HLA Ligand |
| GCGTCAACCAAGTA | | | Atlas² |
| FVDNQYWRY | 41 | HLA-A*01:01 | Previously reported in |
| TTTGTAGATAACCAGTATTGGAGGTAT | 91 | | PXD009602² |
| QLDSAVKNLY | 42 | HLA-A*01:01 | Previously reported in |
| CAACTGGACTCTGCTGT | 92 | | IEDB and HLA Ligand |
| AAAGAACCTGTAC | | | Atlas² |
| EQLKVTTI | 43 | HLA-B*52:01 | Previously reported in |
| GAGCAGCTAAAAGTAACAACAATT | 93 | | IEDB² |
| FRSWAVQTF | 44 | HLA-C*06:02 | Previously reported in |
| TTCCGCAGCTGGGCCGTGCAAACCTTC | 94 | | IEDB and PXD009602² |
| KLYQSIIGY | 45 | HLA-A*03:01 | Previously reported in |
| AAGTTATACCAGAGTATAATAGGATAC | 95 | | IEDB, PXD009602 and |
| | | | PXD014017² |
| SANVSKVSF | 47 | HLA-C*12:02 | Previously reported in |
| AGTGCCAACGTTTCAAAAGTTTCTTTT | 97 | | IEDB, PXD014017, |
| | | | and HLA Ligand Atlas² |
| SQHDILQMI | 48 | HLA-B*13:02 | Previously reported in |
| AGTCAACATGATATTTTACAGATGATA | 98 | | IEDB, PXD009602, |
| | | | and HLA Ligand Atlas² |
| VAKTPRNTY | 49 | HLA-C*12:02 | Previously reported in |
| GTAGCAAAAACGCCCAGGAACACCTAC | 99 | | IEDB² |
| RPRLSAILF | 50 | HLA-B*07:02 | Previously reported in |
| CGGCCACGGCTCAGTGCTATTCTCTTT | 100 | | IEDB, PXD009602, |
| | | | and HLA Ligand Atlas² |
| RVAKTNSLR | 46 | HLA-A*03:01 | Previously reported in |
| CGGGTGGCCAAGACCAACTCCTTACGG | 96 | | IEDB, PXD009602 and |
| | | | PXD014017² |

¹peptides identified in more than one sample may have multiple predicted alleles
²not identified as tumor antigens
IEDB = Immune Epitope Database; PXD = dataset announced via ProteomeXchange

### Example 6: Validation of Putative Tumor-Specific and Tumor-Associated Antigens

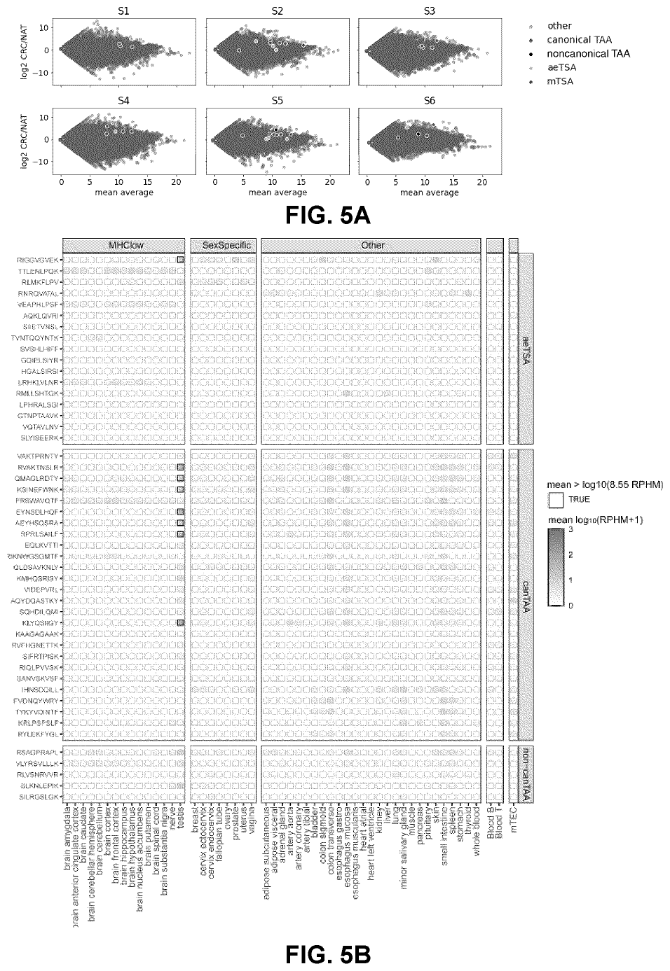

Following the identification of putative TSAs and TAAs, the validation of all the TSAs and a subset of 11 TAAs, which were selected based on favorable initial TMT intensity ratios and precursor ion fractions in cancer vs. matched NAT was performed, prior to validation with synthetic peptides. First, the expression, in TPM, of the source transcripts in their respective tumor samples compared to the matched NAT, as well as the mean average of that transcript in the CRC/NAT sample, were studied (FIG. 5A). This analysis naturally does not include peptides derived from intergenic regions. The average log 2FC for the source transcripts of the putative TSAs and TAAs in the samples in which they were identified was 3.6 and 3.2, respectively. There are a few instances (S2, S6) where the source transcript of an aeTSA is more abundant in the NAT than in the tumor, however this reflects only the overall abundance of the entire transcript, and the peptide coding sequences are in fact more abundant in the cancer. This was also true for aeTSAs, in which the peptide coding region is either entirely absent or lowly expressed in the NAT but is more highly expressed in the cancer tissue.

To evaluate the specificity of the putative tumor antigens, the mean expression of the peptide-coding sequences in the large dataset of healthy tissues provided by the Genotype-Tissue Expression project (GTEx) was determined (FIG. 5B). The TSA sequences are not expressed above 8.55 rphm in any healthy tissues, with the exception of RIGGVGVEK, an aeTSA identified in S2, which is expressed above threshold in the testis (FIG. 5B). This suggests that this TSA could also be classified as a cancer-testis antigen (CTA), a class of aeTSA that is expressed in male germ cells but may also be aberrantly expressed in cancer. Due to the absence of MHC I in testis, these antigens are also promising candidates for cancer immunotherapy (65). This putative TSA is an LY6G6F-LY6G6D exon frameshift. While these genes have not been previously reported as CTAs, another member of the same gene family, LY6K, has been reported as a CTA in lung and esophageal cancers (66). TAA expression was below threshold in healthy tissues, although it tended to be higher in the esophagus and the transverse colon. Seven of these peptides were also expressed above threshold in the testis.

### Example 7: Cancer Specificity and Immunogenicity of TSAs and TAAs

The putative TSAs and TAAs were validated by MS with their corresponding synthetic peptides. These TAAs were selected based on favorable initial TMT intensity ratios and precursor ion fractions in cancer vs matched NAT. These candidates all had MS/MS that correlated well with those of the synthetic peptides, with Pearson correlation score ≥0.6. Synthetic peptides were labelled with TMT10plex-129N, 130N, and 131 at concentrations of 10, 100, and 1000 fmol, respectively, and spiked into remaining purified MAPs from tissue samples that were labeled with TMT126 (NAT) and 127N (CRC). SPS-MS3 was then used to quantify peptides of interest in these samples. Despite the decreased sensitivity of SPS-MS3, it was possible to quantify seven TSAs and seven TAAs. Good quality PSMs were selected for quantification, and all were more abundant in their respective CRC compared to NAT (Table 8). Determining the ratio of intensity of TMT127N peptides compared to TMT126 peptides revealed that TSAs had a median intensity fold change of 16.96 in CRC compared to NAT, while TAAs had a fold change of 6.93. In addition, the TSA with sequence RYLEKFYGL was also overexpressed in the S1 tumor, despite only passing the transcriptomic thresholds for S6. Thus, it was possible to demonstrate that the TSA identification methodology used in this study successfully identified TSA and TAA sequences that are more highly abundant at the surface of cancer cells than that of NAT. TSAs and TAAs candidates that were validated with synthetic peptides are listed in Tables 9A-9B.

TABLE 8
Relative quantification ratios of validated tumor antigens in CRC.

| | | | Endogenous | | SPS-MS3 | Synthetic |
|---|---|---|---|---|---|---|
| | Nature of | | sample | Mean | ratio | calibration |
| Sequence | antigen | Sample | ratio | intensity | (127N/126) | curve R² |
| RMLLSHTGK | aeTSA | RKO | N.D. | N.D. | N.D. | N.D. |
| LPHRALSGI | aeTSA | S1 | −0.364 | N.D. | N.D. | N.D. |
| GTNPTAAVK | aeTSA | S2 | 2.095 | 7238.425242 | 12.174 | 1.000 |
| LRHKLVLNR | aeTSA | S2 | 0.307 | N.D. | N.D. | N.D. |
| RIGGVGVEK | aeTSA | S2 | 1.965 | 29256.45 | 6.740 | 1.000 |
| SIIETVNSL | aeTSA | S2 | 0.288 | N.D. | N.D. | N.D. |
| TVNTQQYNTK | aeTSA | S2 | −0.021 | N.D. | N.D. | N.D. |
| SVSHLHIFF | aeTSA | S3 | −1.100 | N.D. | N.D. | N.D. |
| TTLENLPQK | aeTSA | S4 | 0.134 | 3140.8875 | 3.783 | 0.999 |
| AQKLQVRI | aeTSA | S5 | 0.793 | N.D. | N.D. | N.D. |
| GQIELSIYR | aeTSA | S5 | 0.328 | N.D. | N.D. | N.D. |
| HGALSIRSI | aeTSA | S5 | 0.777 | N.D. | N.D. | N.D. |
| RLMKFLPV | aeTSA | S5 | 0.171 | N.D. | N.D. | N.D. |
| SLYISEERK | aeTSA | S5 | 0.046 | N.D. | N.D. | N.D. |
| VQTAVLNV | aeTSA | S5 | 1.089 | N.D. | N.D. | N.D. |
| VEAPHLPSF | aeTSA | S6 | 1.059 | 43782.84192 | 41.318 | 1.000 |
| RNRQVATAL | aeTSA | S6 | 1.090 | 12174.6625 | 5.722 | 1.000 |
| RNRQVATAL | Not | S1 | 0.890 | 15514.2375 | 3.507 | 1.000 |
| | assigned | | | | | |
| KIGEVIVTK | mTSA | S2 | 2.506 | 70659.6 | 13.637 | 1.000 |
| TRSTIILHL | mTSA | S3 | 1.381 | 34365.32187 | 48.807 | 0.997 |
| VLYRSVLLLK | non- | S6 | 0.997 | N.D. | N.D. | N.D. |
| | canonical | | | | | |
| | TAA | | | | | |
| TYKYVDINTF | canonical | S1 | 1.969 | 29834.36875 | 8.226 | 0.998 |
| | TAA | | | | | |
| RYLEKFYGL | canonical | S1 | 2.840 | 27614.24286 | 7.661 | 0.997 |
| | TAA | | | | | |
| RYLEKFYGL | canonical | S6 | 2.970 | 106928.2875 | 16.090 | 0.999 |
| | TAA | | | | | |
| KSINEFWNK | canonical | S2 | 2.212 | 56110.11667 | 5.238 | 0.999 |
| | TAA | | | | | |
| RIQLPVVSK | canonical | S4 | 1.083 | 7612.378571 | 2.073 | 0.999 |
| | TAA | | | | | |
| QMAGLRDTY | canonical | S3 | 1.140 | 36090.60294 | 2.884 | 0.999 |
| | TAA | | | | | |
| AQYDQASTKY | canonical | S4 | 1.452 | N.D. | N.D. | N.D. |
| | TAA | | | | | |
| FVDNQYWRY | canonical | S4 | 0.721 | 5853.986533 | 10.954 | 1.000 |
| | TAA | | | | | |
| SANVSKVSF | canonical | S5 | 1.114 | 12780.925 | 2.321 | 0.999 |
| | TAA | | | | | |

Tables 9A and 9B depict the TSAs and TAAs candidates that were validated with synthetic peptides.

TABLE 9A
List of TSAs validated with synthetic peptides

| Sequence | SEQ ID NO: |
|---|---|
| RMLLSHTGK | 1 |
| LPHRALSGI | 2 |
| GTNPTAAVK | 3 |
| LRHKLVLNR | 4 |
| RIGGVGVEK | 5 |
| SIIETVNSL | 6 |
| TVNTQQYNTK | 7 |
| SVSHLHIF | 8 |
| TTLENLPQK | 9 |
| AQKLQVRI | 10 |
| GQIELSIYR | 11 |
| HGALSIRSI | 12 |
| RLMKFLPV | 13 |
| SLYISEERK | 14 |
| VQTAVLNV | 15 |
| KIGEVIVTK | 16 |
| TRSTIILHL | 17 |
| RNRQVATAL | 24 |
| VEAPHLPSF | 25 |

TABLE 9B
List of TAAs validated with synthetic peptides

| Sequences | SEQ ID NO: |
|---|---|
| VLYRSVLLLK | 22 |
| TYKYVDINTF | 27 |
| RYLEKFYGL | 29 |
| KSINEFWNK | 31 |
| RIQLPVVSK | 33 |
| QMAGLRDTY | 38 |
| AQYDQASTKY | 40 |
| FVDNQYWRY | 41 |
| SANVSKVSF | 47 |

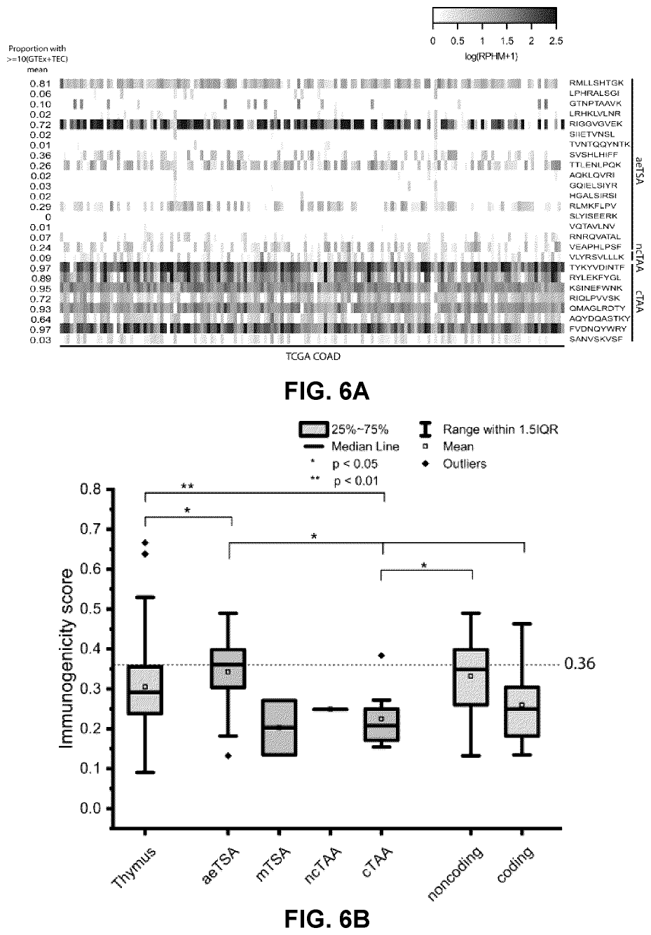

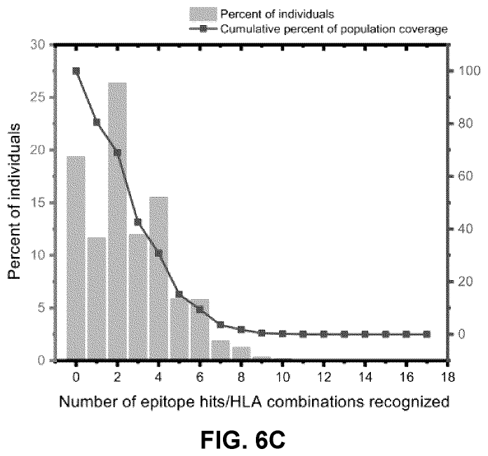

To examine the intertumoral distribution of these TSAs and TAAs in other CRC tumors, the log (RPHM+1) expression of the peptide coding sequences in 151 colon adenocarcinoma samples from The Cancer Genome Atlas (TCGA) (FIG. 6A) were plotted. To evaluate the sharing potential of the antigens, for each peptide of interest, the average of log-transformed (log (rphm+1)) values of pooled GTEx (n=2442) and mTEC (n=8) samples was first calculated. Overall, nine TSAs (53%) and nine TAAs (100%) had an expression ≥10-fold above their corresponding averaged GTEx/mTEC value in at least 5% of TCGA COAD tumors. This demonstrates that TAAs are more frequently shared among COAD TCGA tumors than their TSA counterparts. However, this also means that most TSAs are highly shared in these samples.

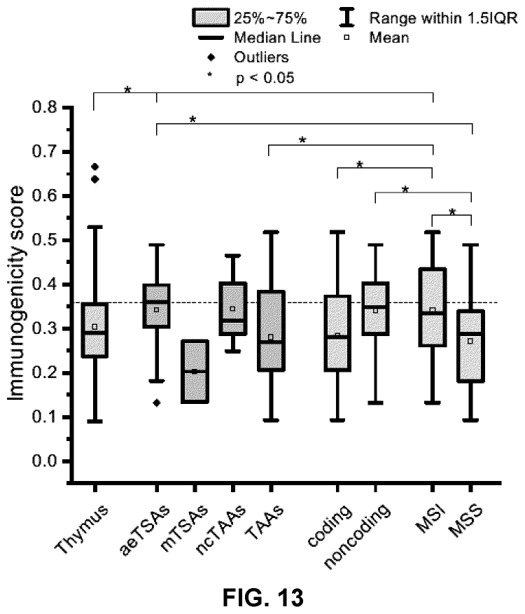

Another important consideration in the identification of tumor antigens is whether these peptides are able to invoke an effective anti-tumor immune response. Repitope predictions of immunogenicity revealed that the aeTSAs are predicted to be significantly more immunogenic than a set of thymic peptides which are presumed non-immunogenic (67) (FIG. 6B). In addition, aeTSAs had significantly higher immunogenicity scores compared to canonical TAAs and to coding TAs overall (TSAs and TAAs derived from coding regions). In fact, TAAs from canonical regions were significantly less immunogenic than thymic peptides, according to these predictions (p<0.01). This could be partially due to the low number of TAAs that were validated. If these predictions were considered with the entire set of 31 TAAs, this is no longer the case (FIG. 13). Considering all 31 TAAs revealed that MSI TAs are predicted to be more immunogenic than thymic peptides, while there is also a statistically significant increase in predicted immunogenicity of TAs derived from MSI tissues compared to MSS.

Finally, an approximation of the proportion of individuals who possess the alleles that are predicted to bind and present the tumor antigens was sought (FIG. 6C). Many of the antigens in the samples are fairly prevalent, and an estimation with the IEDB population coverage tool predicted that 80.64% of the United States population expresses at least one of the alleles associated with the TAs identified in this study.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Bray, F., Ferlay, J., Soerjomataram, I., Siegel, R. L., Torre, L. A., and Jemal, A. (2018) Global cancer statistics 2018: GLOBOCAN estimates of incidence and mortality worldwide for 36 cancers in 185 countries. CA Cancer J Clin 68, 394-424
2. Arnold, M., Sierra, M. S., Laversanne, M., Soerjomataram, I., Jemal, A., and Bray, F. (2017) Global patterns and trends in colorectal cancer incidence and mortality. Gut 66, 683-691
3. Eriksen, A. C., Sorensen, F. B., Lindebjerg, J., Hager, H., dePont Christensen, R., Kjaer-Frifeldt, S., and Hansen, T. F. (2018) The Prognostic Value of Tumor-Infiltrating lymphocytes in Stage II Colon Cancer. A Nationwide Population-Based Study. Transl Oncol 11, 979-987
4. Zhao, Y., Ge, X., He, J., Cheng, Y., Wang, Z., Wang, J., and Sun, L. (2019) The prognostic value of tumor-infiltrating lymphocytes in colorectal cancer differs by anatomical subsite: a systematic review and meta-analysis. World J Surg Oncol 17, 85
5. Le, D. T., Uram, J. N., Wang, H., Bartlett, B. R., Kemberling, H., Eyring, A. D., Skora, A. D., Luber, B. S., Azad, N. S., Laheru, D., Biedrzycki, B., Donehower, R. C., Zaheer, A., Fisher, G. A., Crocenzi, T. S., Lee, J. J., Duffy, S. M., Goldberg, R. M., de la Chapelle, A., Koshiji, M., Bhaijee, F., Huebner, T., Hruban, R. H., Wood, L. D., Cuka, N., Pardoll, D. M., Papadopoulos, N., Kinzler, K. W., Zhou, S., Cornish, T. C., Taube, J. M., Anders, R. A., Eshleman, J. R., Vogelstein, B., and Diaz, L. A., Jr. (2015) PD-1 Blockade in Tumors with Mismatch-Repair Deficiency. N Engl J Med 372, 2509-2520
6. Fabrizio, D. A., George, T. J., Jr., Dunne, R. F., Frampton, G., Sun, J., Gowen, K., Kennedy, M., Greenbowe, J., Schrock, A. B., Hezel, A. F., Ross, J. S., Stephens, P. J., Ali, S. M., Miller, V. A., Fakih, M., and Klempner, S. J. (2018) Beyond microsatellite testing: assessment of tumor mutational burden identifies subsets of colorectal cancer who may respond to immune checkpoint inhibition. J Gastrointest Oncol 9, 610-617
7. Wagner, S., Mullins, C. S., and Linnebacher, M. (2018) Colorectal cancer vaccines: Tumor-associated antigens vs neoantigens. World J Gastroenterol 24, 5418-5432
8. Loffler, M. W., Kowalewski, D. J., Backert, L., Bernhardt, J., Adam, P., Schuster, H., Dengler, F., Backes, D., Kopp, H. G., Beckert, S., Wagner, S., Konigsrainer, I., Kohlbacher, O., Kanz, L., Konigsrainer, A., Rammensee, H. G., Stevanovic, S., and Haen, S. P. (2018) Mapping the HLA Ligandome of Colorectal Cancer Reveals an Imprint of Malignant Cell Transformation. Cancer Res 78, 4627-4641
9. Picard, E., Verschoor, C. P., Ma, G. W., and Pawelec, G. (2020) Relationships Between Immune Landscapes, Genetic Subtypes and Responses to Immunotherapy in Colorectal Cancer. Front Immunol 11, 369
10. Parkhurst, M. R., Yang, J. C., Langan, R. C., Dudley, M. E., Nathan, D. A., Feldman, S. A., Davis, J. L., Morgan, R. A., Merino, M. J., Sherry, R. M., Hughes, M. S., Kammula, U. S., Phan, G. Q., Lim, R. M., Wank, S. A., Restifo, N. P., Robbins, P. F., Laurencot, C. M., and Rosenberg, S. A. (2011) T cells targeting carcinoembryonic antigen can mediate regression of metastatic colorectal cancer but induce severe transient colitis. Mol Ther 19, 620-626
11. Minati, R., Perreault, C., and Thibault, P. (2020) A Roadmap Toward the Definition of Actionable Tumor-Specific Antigens. Front Immunol 11, 583287
12. Smith, C. C., Selitsky, S. R., Chai, S., Armistead, P. M., Vincent, B. G., and Serody, J. S. (2019) Alternative tumour-specific antigens. Nat Rev Cancer 19, 465-478
13. Kloor, M., Reuschenbach, M., Karbach, J., Rafiyan, M., Al-Batran, S.-E., Pauligk, C., Jaeger, E., and Doeberitz, M. v. K. (2015) Vaccination of MSI-H colorectal cancer patients with frameshift peptide antigens: A phase I/IIa clinical trial. Journal of Clinical Oncology 33, 3020-3020
14. van den Bulk, J., Verdegaal, E. M. E., Ruano, D., Ijsselsteijn, M. E., Visser, M., van der Breggen, R., Duhen, T., van der Ploeg, M., de Vries, N. L., Oosting, J., Peeters, K., Weinberg, A. D., Farina-Sarasqueta, A., van der Burg, S. H., and de Miranda, N. (2019) Neoantigen-specific immunity in low mutation burden colorectal cancers of the consensus molecular subtype 4. Genome Med 11, 87
15. Newey, A., Griffiths, B., Michaux, J., Pak, H. S., Stevenson, B. J., Woolston, A., Semiannikova, M., Spain, G., Barber, L. J., Matthews, N., Rao, S., Watkins, D., Chau, I., Coukos, G., Racle, J., Gfeller, D., Starling, N., Cunningham, D., Bassani-Sternberg, M., and Gerlinger, M. (2019) Immunopeptidomics of colorectal cancer organoids reveals a sparse HLA class I neoantigen landscape and no increase in neoantigens with interferon or MEK-inhibitor treatment. J Immunother Cancer 7, 309
16. Laumont, C. M., Vincent, K., Hesnard, L., Audemard, E., Bonneil, E., Laverdure, J. P., Gendron, P., Courcelles, M., Hardy, M. P., Cote, C., Durette, C., St-Pierre, C., Benhammadi, M., Lanoix, J., Vobecky, S., Haddad, E., Lemieux, S., Thibault, P., and Perreault, C. (2018) Noncoding regions are the main source of targetable tumor-specific antigens. Sci Transl Med 10
17. Zhao, Q., Laverdure, J. P., Lanoix, J., Durette, C., Cote, C., Bonneil, E., Laumont, C. M., Gendron, P., Vincent, K., Courcelles, M., Lemieux, S., Millar, D. G., Ohashi, P. S., Thibault, P., and Perreault, C. (2020) Proteogenomics Uncovers a Vast Repertoire of Shared Tumor-Specific Antigens in Ovarian Cancer. Cancer Immunol Res 8, 544-555
18. Ehx, G., Larouche, J. D., Durette, C., Laverdure, J. P., Hesnard, L., Vincent, K., Hardy, M. P., Theriault, C., Rulleau, C., Lanoix, J., Bonneil, E., Feghaly, A., Apavaloaei, A., Noronha, N., Laumont, C. M., Delisle, J. S., Vago, L., Hebert, J., Sauvageau, G., Lemieux, S., Thibault, P., and Perreault, C. (2021) Atypical acute myeloid leukemia-specific transcripts generate shared and immunogenic MHC class-I-associated epitopes. Immunity
19. Bolger, A. M., Lohse, M., and Usadel, B. (2014) Trimmomatic: a flexible trimmer for Illumina sequence data. Bioinformatics 30, 2114-2120
20. Dobin, A., Davis, C. A., Schlesinger, F., Drenkow, J., Zaleski, C., Jha, S., Batut, P., Chaisson, M., and Gingeras, T. R. (2013) STAR: ultrafast universal RNA-seq aligner. Bioinformatics 29, 15-21
21. Bray, N. L., Pimentel, H., Melsted, P., and Pachter, L. (2016) Near-optimal probabilistic RNA-seq quantification. Nat Biotechnol 34, 525-527
22. Szolek, A., Schubert, B., Mohr, C., Sturm, M., Feldhahn, M., and Kohlbacher, O. (2014) OptiType: precision HLA typing from next-generation sequencing data. Bioinformatics 30, 3310-3316
23. Jia, P., Yang, X., Guo, L., Liu, B., Lin, J., Liang, H., Sun, J., Zhang, C., and Ye, K. (2020) MSIsensor-pro: Fast, Accurate, and Matched-normal-sample-free Detection of Microsatellite Instability. Genomics Proteomics Bioinformatics 18, 65-71
24. Love, M. I., Huber, W., and Anders, S. (2014) Moderated estimation of fold change and dispersion for RNA-seq data with DESeq2. Genome Biol 15, 550
25. Zhou, Y., Zhou, B., Pache, L., Chang, M., Khodabakhshi, A. H., Tanaseichuk, O., Benner, C., and Chanda, S. K. (2019) Metascape provides a biologist-oriented resource for the analysis of systems-level datasets. Nat Commun 10, 1523
26. Hardy, M. P., Audemard, E., Migneault, F., Feghaly, A., Brochu, S., Gendron, P., Boilard, E., Major, F., Dieude, M., Hebert, M. J., and Perreault, C. (2019) Apoptotic endothelial cells release small extracellular vesicles loaded with immunostimulatory viral-like RNAs. Sci Rep 9, 7203
27. Karolchik, D., Hinrichs, A. S., Furey, T. S., Roskin, K. M., Sugnet, C. W., Haussler, D., and Kent, W. J. (2004) The UCSC Table Browser data retrieval tool. Nucleic Acids Res 32, D493-496
28. Cingolani, P., Platts, A., Wang Ie, L., Coon, M., Nguyen, T., Wang, L., Land, S. J., Lu, X., and Ruden, D. M. (2012) A program for annotating and predicting the effects of single nucleotide polymorphisms, SnpEff: SNPs in the genome of Drosophila melanogaster strain w1118; iso-2; iso-3. Fly (Austin) 6, 80-92
29. Daouda, T., Perreault, C., and Lemieux, S. (2016) pyGeno: A Python package for precision medicine and proteogenomics. F1000Res 5, 381
30. Lanoix, J., Durette, C., Courcelles, M., Cossette, E., Comtois-Marotte, S., Hardy, M. P., Côté, C., Perreault, C., and Thibault, P. (2018) Comparison of the MHC I immunopeptidome repertoir of B-cell lymphoblasts using two isolation methods. Proteomics 18, e1700251
31. Ma, B., Zhang, K., Hendrie, C., Liang, C., Li, M., Doherty-Kirby, A., and Lajoie, G. (2003) PEAKS: powerful software for peptide de novo sequencing by tandem mass spectrometry. Rapid Commun Mass Spectrom 17, 2337-2342
32. Courcelles, M., Durette, C., Daouda, T., Laverdure, J. P., Vincent, K., Lemieux, S., Perreault, C., and Thibault, P. (2020) MAPDP: A Cloud-Based Computational Platform for Immunopeptidomics Analyses. J Proteome Res 19, 1873-1881
33. Wu, T. D., Reeder, J., Lawrence, M., Becker, G., and Brauer, M. J. (2016) GMAP and GSNAP for Genomic Sequence Alignment: Enhancements to Speed, Accuracy, and Functionality. Methods Mol Biol 1418, 283-334
34. Ogishi, M., and Yotsuyanagi, H. (2019) Quantitative Prediction of the Landscape of T Cell Epitope Immunogenicity in Sequence Space. Front Immunol 10, 827
35. Conway, J. R., Lex, A., and Gehlenborg, N. (2017) UpSetR: an R package for the visualization of intersecting sets and their properties. Bioinformatics 33, 2938-2940
36. Hanzelmann, S., Castelo, R., and Guinney, J. (2013) GSVA: gene set variation analysis for microarray and RNA-seq data. BMC Bioinformatics 14, 7
37. Yoshihara, K., Shahmoradgoli, M., Martinez, E., Vegesna, R., Kim, H., Torres-Garcia, W., Trevino, V., Shen, H., Laird, P. W., Levine, D. A., Carter, S. L., Getz, G., Stemke-Hale, K., Mills, G. B., and Verhaak, R. G. (2013) Inferring tumour purity and stromal and immune cell admixture from expression data. Nat Commun 4, 2612
38. Chan, A. Y., and Anderson, M. S. (2015) Central tolerance to self revealed by the autoimmune regulator. Ann N Y Acad Sci 1356, 80-89
39. Pfammatter, S., Bonneil, E., Lanoix, J., Vincent, K., Hardy, M. P., Courcelles, M., Perreault, C., and Thibault, P. (2020) Extending the Comprehensiveness of Immunopeptidome Analyses Using Isobaric Peptide Labeling. Anal Chem 92, 9194-9204
40. Pira, G., Uva, P., Scanu, A. M., Rocca, P. C., Murgia, L., Uleri, E., Piu, C., Porcu, A., Carru, C., Manca, A., Persico, I., Muroni, M. R., Sanges, F., Serra, C., Dolei, A., Angius, A., and De Miglio, M. R. (2020) Landscape of transcriptome variations uncovering known and novel driver events in colorectal carcinoma. Sci Rep 10, 432
41. Kawakami, H., Zaanan, A., and Sinicrope, F. A. (2015) Microsatellite instability testing and its role in the management of colorectal cancer. Curr Treat Options Oncol 16, 30
42. Jimeno, A., Messersmith, W. A., Hirsch, F. R., Franklin, W. A., and Eckhardt, S. G. (2009) KRAS mutations and sensitivity to epidermal growth factor receptor inhibitors in colorectal cancer: practical application of patient selection. J Clin Oncol 27, 1130-1136
43. Van Cutsem, E., Kohne, C. H., Lang, I., Folprecht, G., Nowacki, M. P., Cascinu, S., Shchepotin, I., Maurel, J., Cunningham, D., Tejpar, S., Schlichting, M., Zubel, A., Celik, I., Rougier, P., and Ciardiello, F. (2011) Cetuximab plus irinotecan, fluorouracil, and leucovorin as first-line treatment for metastatic colorectal cancer: updated analysis of overall survival according to tumor KRAS and BRAF mutation status. J Clin Oncol 29, 2011-2019
44. Ahmed, D., Eide, P. W., Eilertsen, I. A., Danielsen, S. A., Eknaes, M., Hektoen, M., Lind, G. E., and Lothe, R. A. (2013) Epigenetic and genetic features of 24 colon cancer cell lines. Oncogenesis 2, e71
45. Berg, K. C. G., Eide, P. W., Eilertsen, I. A., Johannessen, B., Bruun, J., Danielsen, S. A., Bjornslett, M., Meza-Zepeda, L. A., Eknaes, M., Lind, G. E., Myklebost, O., Skotheim, R. I., Sveen, A., and Lothe, R. A. (2017) Multi-omics of 34 colorectal cancer cell lines—a resource for biomedical studies. Mol Cancer 16, 116
46. Niu, B., Ye, K., Zhang, Q., Lu, C., Xie, M., Mclellan, M. D., Wendl, M. C., and Ding, L. (2014) MSIsensor: microsatellite instability detection using paired tumor-normal sequence data. Bioinformatics 30, 1015-1016
47. Aaltonen, L. A., Peltomaki, P., Mecklin, J. P., Jarvinen, H., Jass, J. R., Green, J. S., Lynch, H. T., Watson, P., Tallqvist, G., Juhola, M., and et al. (1994) Replication errors in benign and malignant tumors from hereditary nonpolyposis colorectal cancer patients. Cancer Res 54, 1645-1648
48. Llosa, N. J., Cruise, M., Tam, A., Wicks, E. C., Hechenbleikner, E. M., Taube, J. M., Blosser, R. L., Fan, H., Wang, H., Luber, B. S., Zhang, M., Papadopoulos, N., Kinzler, K. W., Vogelstein, B., Sears, C. L., Anders, R. A., Pardoll, D. M., and Housseau, F. (2015) The vigorous immune microenvironment of microsatellite instable colon cancer is balanced by multiple counter-inhibitory checkpoints. Cancer Discov 5, 43-51
49. Le, D. T., Durham, J. N., Smith, K. N., Wang, H., Bartlett, B. R., Aulakh, L. K., Lu, S., Kemberling, H., Wilt, C., Luber, B. S., Wong, F., Azad, N. S., Rucki, A. A., Laheru, D., Donehower, R., Zaheer, A., Fisher, G. A., Crocenzi, T. S., Lee, J. J., Greten, T. F., Duffy, A. G., Ciombor, K. K., Eyring, A. D., Lam, B. H., Joe, A., Kang, S. P., Holdhoff, M., Danilova, L., Cope, L., Meyer, C., Zhou, S., Goldberg, R. M., Armstrong, D. K., Bever, K. M., Fader, A. N., Taube, J., Housseau, F., Spetzler, D., Xiao, N., Pardoll, D. M., Papadopoulos, N., Kinzler, K. W., Eshleman, J. R., Vogelstein, B., Anders, R. A., and Diaz, L. A., Jr. (2017) Mismatch repair deficiency predicts response of solid tumors to PD-1 blockade. Science 357, 409-413
50. Hanahan, D., and Weinberg, R. A. (2000) The hallmarks of cancer. Cell 100, 57-70
51. Hanahan, D., and Weinberg, R. A. (2011) Hallmarks of cancer: the next generation. Cell 144, 646-674
52. Prossomariti, A., Piazzi, G., Alquati, C., and Ricciardiello, L. (2020) Are Wnt/beta-Catenin and PI3K/AKT/mTORC1 Distinct Pathways in Colorectal Cancer? Cell Mol Gastroenterol Hepatol 10, 491-506
53. Danaher, P., Warren, S., Dennis, L., D'Amico, L., White, A., Disis, M. L., Geller, M. A., Odunsi, K., Beechem, J., and Fling, S. P. (2017) Gene expression markers of Tumor Infiltrating Leukocytes. J Immunother Cancer 5, 18
54. Barbie, D. A., Tamayo, P., Boehm, J. S., Kim, S. Y., Moody, S. E., Dunn, I. F., Schinzel, A. C., Sandy, P., Meylan, E., Scholl, C., Frohling, S., Chan, E. M., Sos, M. L., Michel, K., Mermel, C., Silver, S. J., Weir, B. A., Reiling, J. H., Sheng, Q., Gupta, P. B., Wadlow, R. C., Le, H., Hoersch, S., Wittner, B. S., Ramaswamy, S., Livingston, D. M., Sabatini, D. M., Meyerson, M., Thomas, R. K., Lander, E. S., Mesirov, J. P., Root, D. E., Gilliland, D. G., Jacks, T., and Hahn, W. C. (2009) Systematic RNA interference reveals that oncogenic KRAS-driven cancers require TBK1. Nature 462, 108-112
55. Kim, H., Jen, J., Vogelstein, B., and Hamilton, S. R. (1994) Clinical and pathological characteristics of sporadic colorectal carcinomas with DNA replication errors in microsatellite sequences. Am J Pathol 145, 148-156
56. Smyrk, T. C., Watson, P., Kaul, K., and Lynch, H. T. (2001) Tumor-infiltrating lymphocytes are a marker for microsatellite instability in colorectal carcinoma. Cancer 91, 2417-2422
57. Dolcetti, R., Viel, A., Doglioni, C., Russo, A., Guidoboni, M., Capozzi, E., Vecchiato, N., Macri, E., Fornasarig, M., and Boiocchi, M. (1999) High prevalence of activated intraepithelial cytotoxic T lymphocytes and increased neoplastic cell apoptosis in colorectal carcinomas with microsatellite instability. Am J Pathol 154, 1805-1813
58. Phillips, S. M., Banerjea, A., Feakins, R., Li, S. R., Bustin, S. A., and Dorudi, S. (2004) Tumour-infiltrating lymphocytes in colorectal cancer with microsatellite instability are activated and cytotoxic. Br J Surg 91, 469-475
59. Boland, C. R., Koi, M., Chang, D. K., and Carethers, J. M. (2008) The biochemical basis of microsatellite instability and abnormal immunohistochemistry and clinical behavior in Lynch syndrome: from bench to bedside. Fam Cancer 7, 41-52
60. Sarkizova, S., Klaeger, S., Le, P. M., Li, L. W., Oliveira, G., Keshishian, H., Hartigan, C. R., Zhang, W., Braun, D. A., Ligon, K. L., Bachireddy, P., Zervantonakis, I. K., Rosenbluth, J. M., Ouspenskaia, T., Law, T., Justesen, S., Stevens, J., Lane, W. J., Eisenhaure, T., Lan Zhang, G., Clauser, K. R., Hacohen, N., Carr, S. A., Wu, C. J., and Keskin, D. B. (2020) A large peptidome dataset improves HLA class I epitope prediction across most of the human population. Nat Biotechnol 38, 199-209
61. Larouche, J. D., Trofimov, A., Hesnard, L., Ehx, G., Zhao, Q., Vincent, K., Durette, C., Gendron, P., Laverdure, J. P., Bonneil, E., Cote, C., Lemieux, S., Thibault, P., and Perreault, C. (2020) Widespread and tissue-specific expression of endogenous retroelements in human somatic tissues. Genome Med 12, 40
62. Cherkasova, E., Scrivani, C., Doh, S., Weisman, Q., Takahashi, Y., Harashima, N., Yokoyama, H., Srinivasan, R., Linehan, W. M., Lerman, M. I., and Childs, R. W. (2016) Detection of an Immunogenic HERV-E Envelope with Selective Expression in Clear Cell Kidney Cancer. Cancer Res 76, 2177-2185
63. Patra, R., Das, N. C., and Mukherjee, S. (2021) Exploring the Differential Expression and Prognostic Significance of the COL11A1 Gene in Human Colorectal Carcinoma: An Integrated Bioinformatics Approach. Front Genet 12, 608313
64. Marcu, A., Bichmann, L., Kuchenbecker, L., Kowalewski, D. J., Freudenmann, L. K., Backert, L., Mühlenbruch, L., Szolek, A., Lübke, M., Wagner, P., Engler, T., Matovina, S., Wang, J., Hauri-Hohl, M., Martin, R., Kapolou, K., Walz, J. S., Velz, J., Moch, H., Regli, L., Silginer, M., Weller, M., Löffler, M. W., Erhard, F., Schlosser, A., Kohlbacher, O., Stevanović, S., Rammensee, H.-G., and Neidert, M. C. (2020) The HLA Ligand Atlas—A resource of natural HLA ligands presented on benign tissues. bioRxiv, 778944
65. Gjerstorff, M. F., Andersen, M. H., and Ditzel, H. J. (2015) Oncogenic cancer/testis antigens: prime candidates for immunotherapy. Oncotarget 6, 15772-15787
66. Ishikawa, N., Takano, A., Yasui, W., Inai, K., Nishimura, H., Ito, H., Miyagi, Y., Nakayama, H., Fujita, M., Hosokawa, M., Tsuchiya, E., Kohno, N., Nakamura, Y., and Daigo, Y. (2007) Cancer-testis antigen lymphocyte antigen 6 complex locus K is a serologic biomarker and a therapeutic target for lung and esophageal carcinomas. Cancer Res 67, 11601-11611
67. Adamopoulou, E., Tenzer, S., Hillen, N., Klug, P., Rota, I. A., Tietz, S., Gebhardt, M., Stevanovic, S., Schild, H., Tolosa, E., Melms, A., and Stoeckle, C. (2013) Exploring the MHC-peptide matrix of central tolerance in the human thymus. Nat Commun 4, 2039
68. Kote, S., Pirog, A., Bedran, G., Alfaro, J., and Dapic, I. (2020) Mass Spectrometry-Based Identification of MHC-Associated Peptides. Cancers (Basel) 12
69. Lin, A., Zhang, J., and Luo, P. (2020) Crosstalk Between the MSI Status and Tumor Microenvironment in Colorectal Cancer. Front Immunol 11, 2039
70. Bonaventura, P., Shekarian, T., Alcazer, V., Valladeau-Guilemond, J., Valsesia-Wittmann, S., Amigorena, S., Caux, C., and Depil, S. (2019) Cold Tumors: A Therapeutic Challenge for Immunotherapy. Front Immunol 10, 168
71. Shihab, H. A., Gough, J., Cooper, D. N., Stenson, P. D., Barker, G. L., Edwards, K. J., Day, I. N., and Gaunt, T. R. (2013) Predicting the functional, molecular, and phenotypic consequences of amino acid substitutions using hidden Markov models. Hum Mutat 34, 57-65
72. Rentzsch, P., Witten, D., Cooper, G. M., Shendure, J., and Kircher, M. (2019) CADD: predicting the deleteriousness of variants throughout the human genome. Nucleic Acids Res 47, D886-D894
73. Sherry, S. T., Ward, M., and Sirotkin, K. (1999) dbSNP-database for single nucleotide polymorphisms and other classes of minor genetic variation. Genome Res 9, 677-679
74. Tate, J. G., Bamford, S., Jubb, H. C., Sondka, Z., Beare, D. M., Bindal, N., Boutselakis, H., Cole, C. G., Creatore, C., Dawson, E., Fish, P., Harsha, B., Hathaway, C., Jupe, S. C., Kok, C. Y., Noble, K., Ponting, L., Ramshaw, C. C., Rye, C. E., Speedy, H. E., Stefancsik, R., Thompson, S. L., Wang, S., Ward, S., Campbell, P. J., and Forbes, S. A. (2019) COSMIC: the Catalogue Of Somatic Mutations In Cancer. Nucleic Acids Res 47, D941-D947
75. Ehx, G., and Perreault, C. (2019) Discovery and characterization of actionable tumor antigens. Genome Med 11, 29
76. Vogel, C., and Marcotte, E. M. (2012) Insights into the regulation of protein abundance from proteomic and transcriptomic analyses. Nat Rev Genet 13, 227-232
77. Xiang, B., Snook, A. E., Magee, M. S., and Waldman, S. A. (2013) Colorectal cancer immunotherapy. Discov Med 15, 301-308
78. Gold, P., and Freedman, S. O. (1965) Demonstration of Tumor-Specific Antigens in Human Colonic Carcinomata by Immunological Tolerance and Absorption Techniques. J Exp Med 121, 439-462
79. Zhou, F. (2009) Molecular mechanisms of IFN-gamma to up-regulate MHC class I antigen processing and presentation. Int Rev Immunol 28, 239-260
80. Deutsch, E. W., Bandeira, N., Sharma, V., Perez-Riverol, Y., Carver, J. J., Kundu, D. J., Garcia-Seisdedos, D., Jarnuczak, A. F., Hewapathirana, S., Pullman, B. S., Wertz, J., Sun, Z., Kawano, S., Okuda, S., Watanabe, Y., Hermjakob, H., MacLean, B., MacCoss, M. J., Zhu, Y., Ishihama, Y., and Vizcaino, J. A. (2020) The ProteomeXchange consortium in 2020: enabling ‘big data’ approaches in proteomics. Nucleic Acids Res 48, D1145-D1152
81. Perez-Riverol, Y., Csordas, A., Bai, J., Bernal-Llinares, M., Hewapathirana, S., Kundu, D. J., Inuganti, A., Griss, J., Mayer, G., Eisenacher, M., Perez, E., Uszkoreit, J., Pfeuffer, J., Sachsenberg, T., Yilmaz, S., Tiwary, S., Cox, J., Audain, E., Walzer, M., Jarnuczak, A. F., Ternent, T., Brazma, A., and Vizcaino, J. A. (2019) The PRIDE database and related tools and resources in 2019: improving support for quantification data. Nucleic Acids Res 47, D442-D450.

1. A tumor antigen peptide (TAP) comprising one of the following amino acid sequences:

| Sequence | SEQ ID NO: |
|---|---|
| SIIETVNSL | 6 |
| RMLLSHTGK | 1 |
| LPHRALSGI | 2 |
| GTNPTAAVK | 3 |
| LRHKLVLNR | 4 |
| RIGGVGVEK | 5 |
| TVNTQQYNTK | 7 |
| SVSHLHIFF | 8 |
| TTLENLPQK | 9 |
| AQKLQVRI | 10 |
| GQIELSIYR | 11 |
| HGALSIRSI | 12 |
| RLMKFLPV | 13 |
| SLYISEERK | 14 |
| VQTAVLNV | 15 |
| KIGEVIVTK | 16 |
| TRSTIILHL | 17 |
| SILRGSLGK | 18 |
| RLVSNRVVR | 19 |
| SLKNLEPIK | 20 |
| RSAGPRAPL | 21 |
| VLYRSVLLLK | 22 |
| KAAGAGAAK | 23 |

or a nucleic acid encoding said TAP.

2. The TAP or nucleic acid of claim 1, wherein the TAP comprises one of the sequences defined in SEQ ID NO: 6, 1-5 and 6-17.

3. The TAP or nucleic acid of claim 1, wherein said TAP binds to (a) an HLA-A*02:01 molecule and comprises the sequence of SEQ ID NO: 6; (b) an HLA-A*03:01 molecule and comprises the sequence of SEQ ID NO:1, 11, or 14; (c) an HLA-A*03:02 molecule and comprises the sequence of SEQ ID NOs: 3, 5, 7, 16 or 23; (d) an HLA-A*11:01 molecule and comprises the sequence of SEQ ID NO:9 or 18; (e) an HLA-A*30:01 molecule and comprises the sequence of SEQ ID NO:19, 20 or 23; (f) an HLA-A*32:01 molecule and comprises the sequence of SEQ ID NO:8; (g) an HLA-B*07:02 molecule and comprises the sequence of SEQ ID NO: 2 or 21; (h) an HLA-B*13:02 molecule and comprises the sequence of SEQ ID NO: 13; (i) an HLA-B*27:05 molecule and comprises the sequence of SEQ ID NO: 4; (i) an HLA-B*52:01 molecule and comprises the sequence of SEQ ID NO: 10, 12, or 15; or (k) an HLA-C*06:02 molecule and comprises the sequence of SEQ ID NO: 17.

4-13. (canceled)

14. The TAP or nucleic acid of claim 1, wherein the TAP is encoded by a sequence located in a non-protein coding region of the genome or a long non-coding RNA.

15. The TAP or nucleic acid of claim 14, wherein said non-protein coding region of the genome is an untranslated transcribed region (UTR), an intron, or an intergenic region.

16-18. (canceled)

19. The nucleic acid of claim 1, wherein the nucleic acid is an mRNA.

20-21. (canceled)

22. A combination comprising at least two of the TAPs or nucleic acids defined in claim 1.

23. A synthetic long peptide (SLP) comprising at least one of the amino acid sequences defined in claim 1, or a nucleic acid encoding said SLP.

24. A vesicle or particle comprising the TAP of claim 1, a nucleic acid encoding said TAP, a combination comprising at least two of the TAPs or nucleic acids, an SLP comprising at least one of the amino acid sequences defined in claim 1, or a nucleic acid encoding said SLP.

25. The vesicle or particle of claim 24, wherein the vesicle is a lipid nanoparticle (LNP).

26. (canceled)

27. A composition comprising the TAP of claim 1, a nucleic acid encoding said TAP, a combination comprising at least two of the TAPs or nucleic acids, an SLP comprising at least one of the amino acid sequences defined in claim 1, or a nucleic acid encoding said SLP, or a vesicle or particle comprising the TAP, nucleic acid, combination or SLP, and a pharmaceutically acceptable carrier.

28. A vaccine comprising the TAP of claim 1, a nucleic acid encoding said TAP, a combination comprising at least two of the TAPs or nucleic acids, an SLP comprising at least one of the amino acid sequences defined in claim 1, or a nucleic acid encoding said SLP, a vesicle or particle comprising the TAP, nucleic acid, combination or SLP, or a composition comprising the TAP, nucleic acid, combination, SLP, vesicle or particle, and an adjuvant.

29-32. (canceled)

33. An isolated cell expressing at its surface major histocompatibility complex (MHC) class I molecules comprising the TAP of claim 1 or a combination of said TAP in their peptide binding groove.

34-35. (canceled)

36. A T-cell receptor (TCR), an antibody or an antigen-binding fragment thereof that specifically recognizes the MHC class I molecules expressed at the surface of the cell of claim 33.

37-41. (canceled)

42. An isolated cell expressing at its cell surface the TCR, antibody or an antigen-binding fragment thereof of claim 36.

43. The isolated cell of claim 42, which is a CD8+ T lymphocyte.

44. A cell population comprising at least 0.5% of the isolated cell as defined in claim 42.

45. A method of treating colorectal cancer in a subject comprising administering to the subject an effective amount of:
(a) a TAP comprising or consisting of one of the amino acid sequences defined in SEQ ID NOs: 1-23 and 25-50, or a synthetic long peptide (SLP) comprising at least one of the sequences set forth in SEQ ID NOs: 1-23 and 25-50;
(b) at least one nucleic acid encoding the TAP, combination thereof or SLP defined in (a);
(c) a vesicle or particle comprising the TAP, combination thereof or SLP defined in (a) or the at least one nucleic acid defined in (b);
(d) a composition comprising the TAP, combination thereof or SLP defined in (a), the at least one nucleic acid defined in (b), or the vesicle or particle defined in (c), and a pharmaceutically acceptable carrier;
(e) a vaccine comprising the TAP, combination thereof or SLP defined in (a), the at least one nucleic acid defined in (b), the vesicle or particle defined in (c), or the composition defined in (d), and an adjuvant;
(f) a cell expressing at its surface major histocompatibility complex (MHC) class I molecules comprising the TAP or combination thereof defined in (a) in their peptide binding groove;
(g) a cell expressing at its cell surface a T-cell receptor (TCR) that specifically recognizes MHC class I molecules expressed at the surface of the cell defined in (f); or
(h) a soluble TCR, an antibody or an antigen-binding fragment thereof that specifically binds to the MHC class I molecules expressed at the surface of the cell defined in (f).

46-48. (canceled)

49. The method of claim 45, further comprising administering at least one additional antitumor agent or therapy to the subject.

50. The method of claim 49, wherein said at least one additional antitumor agent or therapy is a chemotherapeutic agent, immunotherapy, an immune checkpoint inhibitor, radiotherapy or surgery.

51-62. (canceled)
